(19) United States
(12) Patent Application Publication
(10) Pub. No.: US 2026/0234654 A1
(43) Pub. Date: Aug. 13, 2026

(54) COMPOSITIONS AND METHODS FOR IMPROVED PROTEIN TRANSLATION FROM RECOMBINANT CIRCULAR RNAS

(71) Applicant: The Board of Trustees of the Leland Stanford Junior University, Stanford, CA (US)

(72) Inventors: Robert Chen, Stanford, CA (US); Howard Y. Chang, Stanford, CA (US); Chun-Kan Chen, Stanford, CA (US)

(21) Appl. No.: 18/571,967

(22) PCT Filed: Jun. 23, 2022
(86) PCT No.: PCT/US2022/034756

§ 371 Date: Dec. 19, 2023

Related U.S. Application Data
(60) Provisional application No. 63/215,102, filed on Jun. 25, 2021.
(60) Provisional application No. 63/232,324, filed on Aug. 12, 2021.
(60) Provisional application No. 63/320,954, filed on Mar. 17, 2022.
(60) Provisional application No. 63/353,109, filed on Jun. 17, 2022.

(51) Int. Cl. C12N 15/85; C07K 1/00; C12N 15/115

(52) U.S. Cl. CPC C12N 15/85; C07K 1/00; C12N 15/115; C12N 2310/16; C12N 2310/532; C12N 2840/203

(57) ABSTRACT

Provided herein are recombinant circular RNA (circRNA) molecules comprising an internal ribosome entry site (IRES) operably linked to a protein-coding nucleic acid sequence. The IRES may be, for example, a Type I IRES, such as a viral IRES. In some embodiments, the IRES is a synthetic IRES, such as an IRES comprising an aptamer. Methods of producing a protein in vitro or in vivo using the recombinant circRNA molecules are also provided.

### STATEMENT OF RELATED APPLICATIONS

This application claims priority to U.S. Provisional Patent Application No. 63/215,102, filed Jun. 25, 2021, U.S. Provisional Patent Application No. 63/232,324, filed Aug. 12, 2021, U.S. Provisional Patent Application No. 63/320,954, filed Mar. 17, 2022, and U.S. Provisional Patent Application No. 63/353,109, filed Jun. 17, 2022, the entire contents of which are incorporated herein by reference for all purposes.

### STATEMENT REGARDING FEDERALLY-SPONSORED RESEARCH

This invention was made with Government support under contract CA209919 and contract number 5T32GM008412 awarded by the National Institutes of Health. The Government has certain rights in the invention.

### FIELD

The present invention relates to recombinant circular RNA (circRNA) molecules comprising viral and/or synthetic internal ribosome entry sites (IRESs), as well as methods for use thereof.

### SEQUENCE LISTING

The text of the computer readable sequence listing filed herewith, titled “39651-601_SQL_ST25”, created Jun. 23, 2022, having a file size of 11,323,344 bytes, is hereby incorporated by reference in its entirety.

### BACKGROUND

Circular RNAs (circRNAs) are a type of single-stranded RNA which, unlike linear RNA, comprises a covalently closed continuous loop. circRNAs occur naturally in mammalian cells, and play important roles in various biological processes. circRNAs innately possess greater stability and resistance to intra- and extracellular RNAses than mRNAs, making them attractive candidates for delivery of key payloads where long-lasting expression is necessary.

Recently, there has been an interest in using recombinant circRNAs to express a protein of interest, in vitro or in vivo. Introduction of an internal ribosome entry sequence (IRES) into a circular RNA allows translation of a protein encoded by a circRNA. However, IRES elements that exist in nature may or may not support translation from engineered circular RNAs, as IRES elements are often evolved in the context of linear RNA genomes.

Accordingly, there is in the need in the art to identify IRES elements that can drive protein translation from recombinant circRNAs. Further, there is a need for engineered IRES elements that improve the amount and/or duration of protein expression from a circRNA.

### BRIEF SUMMARY

Provided herein are circular RNA molecules comprising an internal ribosome entry sequence (IRES) operably linked to a protein-coding sequence.

For example, in some embodiments, a circular RNA molecule comprises an internal ribosome entry site (IRES) sequence operably linked to a protein-coding sequence; wherein the IRES sequence is a viral sequence; and wherein the protein-coding sequence encodes a non-viral protein. In some embodiments, the molecule comprises a spacer upstream of said IRES.

In some embodiments, the non-viral protein is a mammalian protein. In some embodiments, the non-viral protein is a human protein.

In some embodiments, the IRES is a Type 1 IRES. In some embodiments, the IRES is an enterovirus IRES. In some embodiments, the IRES is a human rhinovirus (HRV) IRES.

In some embodiments, the IRES is any one of the IRES listed in Table 7. In some embodiments, the IRES is any one of the following IRES: iEMCV, iHCV, iCVB5, iSwineVesicular, iHRV-A2, iHRV-C3, iHRV-C11, iCVB1, iPV2, iHRV-B17, iEchoV-E15, iEV71, iHRV-A9, iSiminanV4, iEV-D94, iSimianA5, iPV3, iHRV-C54, iHRV-A100, iHRV-B37, iHRV-B4, iHRV-B92, iHRV-B3, iHRV-A1, iEV107, or a fragment or derivative thereof. In some embodiments, the IRES is any one of the following IRES: iEV-B83, iHRV-A57, iHRV-B35, iHRV-B4, iEV-D68, iHRVB_R93, iHRV-B5, iHRVB-B52, iHRVB-B93, iHRV-B84, iHRV-B83_SC2220, iHRV-B72, iHRV-B69, iHRVB_SC0739, iHRV-B91, iHRV-B42, iHRV-B6, iHRV-B83, iHRV-B48, iHRV-B99, iHRV-B79, iHRV-B97, iHRV-B27, iHRVB_3039, iHRVB-B14, iCosV-B1, or a fragment or derivative thereof. In some embodiments, the IRES is iCVB3, or a fragment or derivative thereof. In some embodiments, the IRES is iHRV-B3, or a fragment or derivative thereof.

Also provided herein is a circular RNA molecule comprising a synthetic internal ribosome entry site (IRES) sequence operably linked to a protein-coding sequence. In some embodiments, the IRES is upstream of the protein-coding sequence. In some embodiments, the synthetic IRES sequence comprises an aptamer. In some embodiments, synthetic IRES sequence comprises an aptamer and a second aptamer.

In some embodiments, the aptamer is a wildtype aptamer. In some embodiments, the aptamer is an aptamer was designed and/or evolved to bind one or more DNA sequences. In some embodiments, the aptamer is a mutant aptamer. In some embodiments, the aptamer is modified to have an extended stem region.

In some embodiments, the aptamer is positioned within the secondary structure of the IRES so that is spatially proximal to portion of the IRES responsible for translation initiation. In some embodiments, the aptamer does not interrupt the native eIF4G binding site of the IRES and does not interrupt a native GRNA tetraloop within the IRES.

In some embodiments, the aptamer is an eIF4G-binding aptamer. In some embodiments, the eIF4G-binding aptamer comprises or is encoded by the sequence of SEQ ID NO: 99. In some embodiments, the IRES is a Type 1 IRES. In some embodiments, the IRES is a modified enterovirus IRES. In some embodiments, the IRES is a modified human rhinovirus (HRV) IRES. In some embodiments, the IRES comprises or is encoded by the sequence of any one of SEQ ID NO: 125-129.

In some embodiments, synthetic IRES sequence is a modified iCVB3 IRES. In some embodiments, modified iCVB3 IRES comprises an aptamer inserted in domain I, II, III, IV, V, VI or VII thereof. In some embodiments, the modified iCVB3 IRES comprises an aptamer inserted in domain IV thereof. In some embodiments, the modified iCVB3 aptamer is modified to have an extended stem region. In some embodiments, the modified iCVB3 aptamer is positioned within the secondary structure of the IRES so that is spatially proximal to portion of the IRES responsible for translation initiation. In some embodiments, the modified iCVB3 aptamer does not interrupt the native eIF4G binding site of the IRES and does not interrupt a native GRNA tetraloop within the IRES.

In some embodiments, the synthetic IRES sequence is a modified iHRV-B3 IRES. In some embodiments, the modified iHRV-B3 IRES comprises an aptamer inserted in domain I, II, III, IV, V, or VI thereof. In some embodiments, the modified iHRV-B3 IRES comprises an aptamer inserted in domain IV thereof. In some embodiments, the modified iHRV-B3 IRES aptamer is modified to have an extended stem region. In some embodiments, the modified iHRV-B3 IRES aptamer is positioned within the secondary structure of the IRES so that is spatially proximal to portion of the IRES responsible for translation initiation. In some embodiments, the modified iHRV-B3 aptamer does not interrupt the native eIF4G binding site of the IRES and does not interrupt a native GRNA tetraloop within the IRES.

In some embodiments, the circular RNA comprises a least one 2-thiouridine (2ThioU) or at least one 2′-O-methylcitidine (2OMeC). In some embodiments, the circular RNA molecule comprises about 2% to about 5% 2-thiouridine (e.g., about 2.5% 2-thiouridine). In some embodiments, the circular RNA molecule comprises about 2% to about 5% 2′-O-methylcitidine (e.g., about 2.5% 2′-O-methylcitidine).

Also provided is a nucleic acid that encodes one or more of the circular RNA molecules described herein.

Also provided is a composition comprising one or more of the circular RNA molecules and/or the nucleic acids described herein.

Also provided are host cells comprising one or more of the circular RNA molecules and/or the nucleic acids described herein.

Also provided are methods for producing a protein in a cell, the method comprising contacting a cell with a circular RNA molecule or a nucleic acid described herein under conditions whereby the protein-coding nucleic acid sequence of the circular RNA is translated and the protein is produced in the cell.

Also provided are methods for producing a protein in vitro, the method comprising contacting a cell-free extract with a circular RNA molecule or a nucleic acid under conditions whereby the protein-coding nucleic acid sequence of the circular RNA is translated and the protein is produced.

These and other embodiments will be described in further detail below, and in the appended drawings.

### BRIEF DESCRIPTION OF THE DRAWINGS

FIG. 1 is a graph that shows normalized luminescence (relative to iCVB3) observed in a cell-based screen of viral IRES sequences. The exogenously delivered recombinant circRNA was produced by an in vitro transcription and circularization utilizing circRNA DNA plasmids, with a nanoluciferase reporter operably linked or driven by indicated IRES. n=3 biological replicates. The dotted line represents expression level produced by iCVB3.

FIG. 2 is a graph that shows normalized luminescence (relative to mock-cell extracts, which comprise cell-free extract but do not include any DNA plasmid template encoding a circRNA) observed in a cell-free protein translation screen of rhinovirus type B (HRV-B) and enterovirus B (EV) IRES sequences utilizing recombinant nano-luciferase reporter circRNAs each with specified IRES. n=3 biological replicates. The dotted line represents expression level produced by iCVB3.

FIG. 3 is a graph that shows normalized luminescence (relative to iCVB3) observed in a cell-based screen of viral IRES sequences in different cell types. n=3 biological replicates. The dotted line represents expression level produced by iCVB3. Unless indicated with the numbers in parentheses, all IRESs are type 1.

FIG. 4 is a graph that shows normalized luminescence (relative to iCVB3) observed for various IRES sequences, when tested in different cell lines, highlighting IRES that show various levels of cell specific IRES activity. Normalized fold/iCVB3 IRES expression mean SEM are shown. n=3 biological replicates. The dotted line represents expression level produced by iCVB3.

FIG. 5A shows the structure of the wildtype CVB3 IRES, and locations where an eIF4G-recuriting aptamer (eIF4G) was inserted (labeled 01 through 11). FIG. 5B is a graph that shows normalized luminescence (relative to mock-transfected cells) observed after transfection of cells with circRNAs comprising an aptamer sequence. Mean luminescence fold/mock ±SEM are shown. n=3 biological replicates.

FIG. 6A shows key elements in the structure of the wildtype HRV-B3 TRES and locations were an eiF4G-recuriting aptamer was inserted. FIG. 6B shows variations made to the aptamer to modulate its activity, and the effect of those modifications on luciferase expression. Mean normalized luminescence fold/mock ±SEM are shown. n=3 biological replicates.

FIG. 7A shows NanoLuc activity at 24 hours after transfection of HeLa cells with circRNAs containing deletions of different IRES domains starting from the 5′ end. Secondary structure and truncation points are indicated on the diagram. Data shown are mean±SEM for n=3 biological replicates. * P<0.05 by unpaired t-test compared to full-length (FL) iCVB3.

FIG. 7B shows NanoLuc activity at 24 hours after transfection of HeLa cells with circRNAs containing successive l0 bp deletions starting from the 3′ end of the IRES, immediately prior to the AUG start codon. Data shown are mean±SEM for n=3 biological replicates.

FIG. 7C shows NanoLuc activity at 24 hours after transfection of HeLa cells with circRNAs containing successive 10 nt deletions starting from the 3′ end of the IRES, immediately prior to the AUG start codon. NanoLuc activity was normalized to constitutive firefly luciferase activity from the same sample, then divided by values from mock transfection. Data shown are mean±SEM for n=3 biological replicates.

FIG. 7D shows correlations between the indicated properties and NanoLuc activity at 24 hours after transfection of HeLa cells with circRNAs containing different N-terminal leader sequences between the AUG start codon and NanoLuc reporter. Data shown are mean±SEM for n=3 biological replicates.

FIG. 8 shows NanoLuc activity after transfection of HeLa cells with circRNAs containing either a 3′ or 5′ IRES and spacer sequences of varying lengths. Data shown are mean±SEM for n=3 biological replicates.

FIG. 9 shows NanoLuc activity at 24 hours after transfection of HeLa cells with circRNAs containing the indicated number of stop codons. Data shown are mean±SEM for n=3 biological replicates.

FIG. 10A shows NanoLuc activity at 24 hours after transfection of HeLa cells with circRNAs containing an eIF4G-recruiting aptamer (Apt-eIF4G), shown in inset. Apt-eIF4G was inserted into iCVB3 at 11 different positions as indicated in the schematicData shown are mean±SEM for n=3 biological replicates. *** P<0.001 by unpaired t-test compared to wild-type iCVB3.

FIG. 10B shows mNeonGreen fluorescence at 24 hours after electroporation of HeLa cells with mRNA or circRNAs containing successive optimizations. Data shown are histograms for n>50,000 live singlet cells per condition and mean±SEM for n=3 biological replicates. ** P<O.01, ** * P<0.001 by unpaired two-sided t-test.

FIG. 10C shows the gating strategy to analyze live singlet HEK293T cells after electroporation.

FIG. 11 shows that eIF4G-binding site deletions are translation-lethal and irrecoverable. NanoLuc activity at 24 hours after transfection of HeLa cells with circRNAs containing wild-type iCVB3, iCVB3 with Apt-eIF4G insertion, iCVB3 with eIF4G footprint deletions, or iCVB3 with eIF4G footprint deletions and attempted rescue with Apt-eIF4G. Sub-domain deletions (v1-v4) differed in the position where the stem loop was truncated, but at a minimum all ablated the eIF4G footprint. Data shown are mean±SEM for n=3 biological replicates.

FIG. 12 shows NanoLuc activity at 24 hours after transfection of HeLa, HepG2, and HEK293T cells with circRNAs containing the indicated IRESs. Data shown are mean±SEM for n=3 biological replicates.

FIG. 13A shows NanoLuc activity after in vitro transcription-translation (IVTT) of circRNA plasmids containing enterovirus (EV) or human rhinovirus B (HRV-B) IRESs. All known EV and HRV-B IRES sequences were cloned into circRNA plasmids. Purified plasmids were then subjected to IVTT using HeLa lysate. Data shown are mean±SEM for n=4 biological replicates.

FIG. 13B shows NanoLuc activity at 24 hours after transfection of HeLa cells with circRNAs or linear RNAs containing strong IRESs from the IVTT-based screen. Linear RNA sequences were identical to those of circRNAs with the exclusion of self-splicing introns. Data shown are mean±SEM for n=3 biological replicates.

FIG. 13C shows NanoLuc activity at 24 hours after transfection of HeLa, HepG2, HEK293T, and KG-1 cells with circRNAs containing the indicated IRESs. Values for HeLa, HepG2, and HEK293T cells are the same as in FIG. 12. Data shown are mean±SEM for n=3 biological replicates.

FIG. 14A shows NanoLuc activity after in vitro transcription-translation (IVTT) of circRNA plasmids containing shuffled IRESs. DNA shuffling was performed on human rhinovirus IRESs by fragmenting IRESs and cloning the resulting pool into circRNA plasmids. Purified plasmids were then subjected to IVTT using HeLa lysate. NanoLuc activity was divided by values from mock IVTT. Data shown are mean±SEM for n=4 biological replicates. P<0.05, **P=0.0095, ****P<0.0001 by unpaired two-sided t-test compared to wild-type iHRV-B3.

FIG. 14B shows NanoLuc activity at 24 hours after transfection of HeLa cells with circRNAs containing different insertions of Apt-eIF4G into an IRES of indeterminate structure (iHRV-B3). The putative secondary structure for iHRV-B3, predicted eIF4G and eIF4A binding sites, and locations of Apt-eIF4G insertions are shown. Versions (vi-v6) of each insertion were designed with different stem lengths. Double aptamer refers to insertion of Apt-eIF4G at both the distal and proximal loops. Data shown are mean±SEM for n=3 biological replicates. *P=0.0422, **P=0.0018, ***P=0.0003, ****P<0.0001 by unpaired t-test compared to wild-type iHRV-B3.

FIG. 14C shows sequences of shuffled IRESs.

FIG. 15A shows that RNA modifications 2-thiouridine and 2′-O-methylcytidine do not inhibit circular RNA (circRNA) translation. The listed modifications were incorporated into circRNA during synthesis at 10% incorporation level to assess potential inhibition of translation. m⁶A=n⁶-methyladenosine, 5m=5-methyl, 5mo=5-methoxy, 5-hydroxymethyl, 2ThioU=2-thiouridine, ψ=pseudouridine, N1ψ=N1-methylpseudouridine, N1ethψ=N1-ethylpseudouridine, 2′Fd=2′-fluoro-2′-deoxy, 2′OMeC=2′-O-Methylcytidine.

FIG. 15B shows results of a small-scale titration experiment which revealed that 2-thiouridine and 2′-O-methylcytidine at 2.5% incorporation levels show improved circRNA translation over unmodified or 5% m⁶A. Mean normalized luminescence fold/mock ±SEM are shown (n=3 biological replicates).

FIG. 16A is a graph demonstrating that, at an optimized incorporation level identified previously, 2-thiouridine and 2′-O-methylcytidine improve circRNA translation. CircRNAs were transfected into HeLa cells and Nanoluciferase expression was assayed and normalized to constitutive expression of Firefly Luciferase. Mean normalized luminescence fold/mock ±SEM are shown (n=3 biological replicates). ***p<0.001, unpaired t-test, comparing to unmodified normalized luminescence.

FIG. 16B provides images showing that the RNA modifications N⁶-methyladenosine, 2-thiouridine, and 2′-O-methylcytidine all confer resistance to RNAse degradation.

FIG. 16C shows NanoLuc activity after transfection of HeLa cells with unmodified circRNA or circRNA containing 5% m6A. NanoLuc activity was normalized to constitutive firefly luciferase activity from the same sample, then divided by values from mock transfection. Data shown are mean±SEM for n=3 biological replicates.

FIG. 16D shows mNeonGreen fluorescence at 24 hours after electroporation of HeLa cells with unmodified circRNA or circRNA containing 5% m6A. Mean mNeonGreen expression was measured by flow cytometry and normalized by values from mock electroporation. Data shown are histograms for n>50,000 live singlet cells per condition and mean±SEM for n=3 biological replicates.

FIG. 17A shows NanoLuc activity at 24 hours after transfection of HeLa cells with circRNAs containing 10% incorporation of different RNA modifications. Data shown are mean±SEM for n=3 biological replicates. m⁶A, N⁶-methyladenosine; 5mC, 5-methylcytidine; 5mU, 5-methyluridine; 5moC, 5-methoxycytidine; 5moU, 5-methoxyuridine; 5hmC, 5-hydroxymethylcytidine; 5hmU, 5-hydroxymethyluridine; 2ThioU, 2-thiouridine; ψ, pseudouridine; N1ψ, N¹-methylpseudouridine; N1ethψ, N¹-ethylpseudouridine; 2′FdC, 2′-fluoro-2′-deoxycytidine; 2′FdU, 2′-fluoro-2′-deoxyuridine; 2′OMeC, 2′-O-Methylcytidine.

FIG. 17B shows quantification of circRNA levels in HeLa cells at 24 hours after transfection with circRNAs containing the indicated RNA modifications. Data shown are mean SEM for n=3 biological replicates.

FIG. 17C shows resistance of mRNA and circRNAs with indicated RNA modifications to degradation in escalating doses of fetal bovine serum (FBS). RNAs were incubated in the indicated percent concentrations of FBS at 37° C. for 30 minutes, then briefly denatured in RNA loading buffer before gel electrophoresis. The same amount of ladder per gel and RNA per well were used to allow for comparisons between gels.

FIG. 17D shows NanoLuc activity in supernatant after electroporation of HeLa cells with circRNA or mRNA encoding secreted NanoLuc. CircRNA was synthesized with 5% m6A incorporation and the HRV-B3 IRES. mRNA was synthesized with CleanCap reagent, 100% N1T incorporation, and a 120 nt poly(A) tail. At the indicated hours (h) and days (d) post-electroporation, media was harvested to assay secreted NanoLuc and replaced. Data shown are mean±SEM for n=3 biological replicates.

FIG. 18A shows that additional stop codons do not change circRNA or proteion size. TapeStation gel electrophoresis depicting the size of circRNAs encoding NanoLuc and possessing the indicated number of stop codons.

FIG. 18B shows a Western blot depicting NanoLuc protein in HeLa lysate at 24 hours after electroporation with circRNAs encoding NanoLuc and possessing the indicated number of stop codons. Each lane was loaded with 10 g of total protein.

FIG. 19. In silico RNA structure prediction can inform IRES engineering. RNA structure predictions for synthetic IRESs synIRES01-11 at the site of aptamer insertion. For inter-domain insertions (synIRES01, 03, 05, 09, and 11), structure prediction was performed on Apt-eIF4G and the adjacent iCVB3 domains. For loop insertions (synIRES02, 04, 06, 07, 08, and 10), structure prediction was performed on Apt-eIF4G and the iCVB3 domain containing the insertion. In each structure, nucleotides corresponding to Apt-eIF4G are shown in white.

### DETAILED DESCRIPTION

Protein translation in eukaryotic cells typically relies on the m⁷G cap present at the 5′ end of mRNAs. However, several cap-independent translation mechanisms have been identified. For example, some viral mRNAs employ alternative mechanisms of translation initiation based on internal ribosome entry via an internal ribosome entry sequence (IRES). Cap-independent translation of proteins typically suffers from lower translation strength, as compared to cap-dependent (mRNA translation).

Provided herein are viral and synthetic IRES that can drive expression of a protein (e.g., a non-viral protein) from a circular RNA. The viral and synthetic IRES described herein satisfy an unmet need in the field of cap-independent translation. The IRESs identified may also be used for polycistronic mRNA gene delivery. Because the IRESs described herein drive expression at a wide range of strengths and some in a cell type-dependent manner, the choice of IRES can be used to independently control expression levels of the two or more proteins in a single transcript. This expression level tunability offers an additional layer of control over just dosing leveling.

### Definitions

To facilitate an understanding of the present technology, a number of terms and phrases are defined below. Additional definitions are set forth throughout the detailed description.

The use of the terms “a” and “an” and “the” and “at least one” and similar referents in the context of describing the invention (especially in the context of the following claims) are to be construed to cover both the singular and the plural, unless otherwise indicated herein or clearly contradicted by context.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Nomenclature for nucleotides, nucleic acids, nucleosides, and amino acids used herein is consistent with International Union of Pure and Applied Chemistry (IUPAC) standards (see, e.g., bioinformatics.org/sms/iupac.html).

When referring to a nucleic acid sequence or protein sequence, the term “identity” is used to denote similarity between two sequences. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection. Another algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

The terms “internal ribosome entry site,” “internal ribosome entry sequence,” “IRES” and “IRES sequence region” are used interchangeably herein and refer to cis elements of viral or human cellular RNAs (e.g., messenger RNA (mRNA) and/or circRNAs) that bypass the steps of canonical eukaryotic cap-dependent translation initiation. The canonical cap-dependent mechanism used by the vast majority of eukaryotic mRNAs requires an m⁷G cap at the 5′ end of the mRNA, initiator Met-tRNAmet, more than a dozen initiation factor proteins, directional scanning, and GTP hydrolysis to place a translationally competent ribosome at the start codon. IRESs typically are comprised of a long and highly structured 5′-UTR which mediates the translation initiation complex binding and catalyzes the formation of a functional ribosome.

“Aptamers” are short, single-stranded DNA or RNA molecules that can selectively bind to a specific target. The target may be, for example, a protein, peptide, carbohydrate, small molecule, toxin, or a live cell. Some aptamers can bind DNA, RNA, self-aptamers or other non-self aptamers. Aptamers assume a variety of shapes due to their tendency to form helices and single-stranded loops. Illustrative DNA and RNA aptamers are listed in the Aptamer database (scicrunch.org/resources/Any/record/nlx_144509-1/SCR_001781/resolver?q=*&l=).

The terms “coding sequence,” “coding sequence region,” “coding region,” and “CDS” when referring to nucleic acid sequences may be used to refer to the portion of a DNA or RNA sequence, for example, that is or may be translated to protein. The terms “reading frame,” “open reading frame,” and “ORF,” may be used herein to refer to a nucleotide sequence that begins with an initiation codon (e.g., ATG) and, in some embodiments, ends with a termination codon (e.g., TAA, TAG, or TGA). Open reading frames may contain introns and exons, and as such, all CDSs are ORFs, but not all ORF are CDSs.

The terms “complementary” and “complementarity” refers to the relationship between two nucleic acid sequences or nucleic acid monomers having the capacity to form hydrogen bond(s) with one another by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., about 50%, about 60%, about 70%, about 80%, about 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., at least 65%, at least 70%, at least 75%, at least 80%, at least 85%, at least 90%, at least 95%, at least 97%, at least 98%, at least 99%, or 100%) over a region of at least 8 nucleotides (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides), or if the two nucleic acid sequences hybridize under at least moderate, or, in some embodiments high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook, J., Molecular Cloning: A Laboratory Manual, Cold Spring Harbor Laboratory Press; 4th edition (Jun. 15, 2012). High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS, and 10% dextran sulfate at 42° C., with washes at (i) 42° C. in 0.2×SSC, (ii) 55° C. in 50% formamide, and (iii) 55° C. in 0.1×SSC (optionally in combination with EDTA). Additional details and an explanation of stringency of hybridization reactions are provided in, e.g., Sambrook, supra; and Ausubel et al., eds., Short Protocols in Molecular Biology, 5th ed., John Wiley & Sons, Inc., Hoboken, N.J. (2002). The term “hybridization” or “hybridized” when referring to nucleic acid sequences is the association formed between and/or among sequences having complementarity.

The term “secondary structure,” or “secondary structure element” or “secondary structure sequence region” as used herein in reference to nucleic acid sequences (e.g., RNA, DNA, etc), refers to any non-linear conformation of nucleotide or ribonucleotide units. Such non-linear conformations may include base-pairing interactions within a single nucleic acid polymer or between two polymers. Single-stranded RNA typically forms complex and intricate base-pairing interactions due to its increased ability to form hydrogen bonds stemming from the extra hydroxyl group in the ribose sugar. Examples of secondary structures or secondary structure elements include but are not limited to, for example, stem-loops, hairpin structures, bulges, internal loops, multiloops, coils, random coils, helices, partial helices and pseudoknots. In some embodiments, the term “secondary structure” may refer to a SuRE element. The term “SuRE” stands for stem-loop structured RNA element (SuRE).

The term “free energy,” as used herein, refers to the energy released by folding an unfolded polynucleotide (e.g., RNA or DNA, etc.) molecule, or, conversely, the amount of energy that must be added in order to unfold a folded polynucleotide (e.g., RNA or DNA, etc.) The “minimum free energy (MFE)” of a polynucleotide (e.g., DNA, RNA, etc.) describes the lowest value of free energy observed for the polynucleotide when assessed for various secondary structures thereof. The MFE of an RNA molecule may be used to predict RNA or DNA secondary structure and is affected by the number, composition, and arrangement of the RNA or RNA nucleotides. The more negative free energy a structure has, the more likely is its formation since more stored energy is released by formation of the structure.

The term “melting temperature (Tm)” refers to the temperature at which about 50% of double-stranded nucleic acid structures (e.g., DNA/DNA, DNA/RNA, or RNA/RNA duplexes) denature and dissociate to single-stranded structures.

The term “recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Alternatively, DNA sequences encoding RNA that is not translated may also be considered recombinant. Thus, the term “recombinant” nucleic acid also refers to a nucleic acid which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, the artificial combination may be performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may comprise a naturally occurring amino acid sequence.

The terms “operably linked” and “operatively linked,” as used herein, refer to an arrangement of elements that are configured so as to perform, function or be structured in such a manner as to be suitable for an intended purpose. For example, a given promoter operably linked to a coding sequence is capable of effecting the expression of the coding sequence when the proper enzymes are present. Expression is meant to include the transcription of any one or more of a recombinant nucleic acid encoding a circular RNA, or mRNA from a DNA or RNA template and can further include translation of a protein from a recombinant circular RNA comprising an IRES sequence (e.g., a non-native IRES). Thus, for example, intervening untranslated yet transcribed sequences can be present between a promoter sequence and a coding sequence and the promoter sequence can still be considered to be “operably linked” to the coding sequence.

### Circular RNAs

The instant disclosure provides recombinant circular RNA molecules comprising an internal ribosome entry site (IRES) sequence operably linked to a protein-coding sequence, and DNA sequences encoding the same. In some embodiments, the protein coding sequence encodes a non-viral protein. For example, in some embodiments, the protein coding sequence encodes an animal protein, a plant protein, a bacterial protein, a fungal protein, or an artificial protein. In some embodiments, the protein coding sequence encodes a mammalian protein, such as a human protein.

Recombinant circRNA molecules may be generated or engineered according to several methods. For example, recombinant circRNA molecules may be generated by back-splicing of linear RNAs. For example, in some embodiments, a recombinant circular RNA is produced by back-splicing of a downstream 5′ splice site (splice donor) to an upstream 3′ splice site (splice acceptor). The splice donor and/or splice acceptor may be found, for example, in a human intron or portion thereof that is typically used for circRNA production at endogenous loci. In some embodiments, a recombinant circular RNA is produced by contacting a cell with a DNA plasmid, wherein the DNA plasmid encodes a linear RNA, and the linear RNA is back-spliced to produce a recombinant circular RNA. In some embodiments, the DNA plasmid comprises introns from the mammalian ZKSCAN1 gene.

In some embodiments, circular RNAs can be generated by a non-mammalian splicing method. For example, linear RNAs containing various types of introns, including self-splicing group I introns, self-splicing group II introns, spliceosomal introns, and tRNA introns can be circularized. In particular, group I and group II introns have the advantage that they can be readily used for production of circular RNAs in vitro as well as in vivo because of their ability to undergo self-splicing due to their autocatalytic ribozyme activity.

Alternatively, circular RNAs can be produced in vitro from a linear RNA by chemical or enzymatic ligation of the 5′ and 3′ ends of the RNA. Chemical ligation can be performed, for example, using cyanogen bromide (BrCN) or ethyl-3-(3′-dimethylaminopropyl) carbodiimide (EDC) for activation of a nucleotide phosphomonoester group to allow phosphodiester bond formation (Sokolova, FEBSLett, 232:153-155 (1988); Dolinnaya et al., Nucleic Acids Res., 19: 3067-3072 (1991); Fedorova, Nucleosides Nucleotides Nucleic Acids, 15: 1137-1147 (1996)). Alternatively, enzymatic ligation can be used to circularize RNA. Exemplary ligases that can be used include T4 DNA ligase (T4 Dnl), T4 RNA ligase 1 (T4 Rnl 1), and T4 RNA ligase 2 (T4 Rnl 2).

In some embodiments, splint ligation may be used to generate circular RNA. Splint ligation involves the use of an oligonucleotide splint that hybridizes with the two ends of a linear RNA to bring the ends of the linear RNA together for ligation. Hybridization of the splint, which can be either a deoxyribo-oligonucleotide or a ribooligonucleotide, orients the 5′-phosphate and 3′-OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, is more efficient in some cases than enzymatic ligation if the structure of the hybridized splint-RNA complex interferes with enzymatic activity (see, e.g., Dolinnaya et al. Nucleic Acids Res, 21(23): 5403-5407 (1993); Petkovic et al., Nucleic Acids Res, 43(4): 2454-2465 (2015)).

While circular RNAs generally are more stable than their linear counterparts, primarily due to the absence of free ends necessary for exonuclease-mediated degradation, additional modifications may be made to the recombinant circRNA described herein to further improve stability. Still other kinds of modifications may improve circularization efficiency, purification of circRNA, and/or protein expression from circRNA. For example, the recombinant circRNA may be engineered to include “homology arms” (i.e., 9-19 nucleotides in length placed at the 5′ and 3′ ends of a precursor RNA with the aim of bringing the 5′ and 3′ splice sites into proximity of one another), spacer sequences, and/or a phosphorothioate (PS) cap (Wesselhoeft et al., Nat. Commun., 9: 2629 (2018)). The recombinant circRNA also may be engineered to include 2′-O-methyl-, -fluoro- or -O-methoxyethyl conjugates, phosphorothioate backbones, or 2′,4′-cyclic 2′-O-ethyl modifications to increase the stability thereof (Holdt et al., Front Physiol., 9: 1262 (2018); Krutzfeldt et al., Nature, 438(7068): 685-9 (2005); and Crooke et al., Cell Metab., 27(4): 714-739 (2018)). The recombinant circRNA molecule also may comprise one or more modifications that reduce the innate immunogenicity of the circRNA molecule in a host, such as at least one N6-methyladenosine (m⁶A).

In some embodiments, the recombinant circRNA molecule comprises at least one 2-thiouridine (2ThioU) or at least one 2′-O-methylcytidine (2OMeC). 2-thiouridine is a modified nucleobase found in tRNAs that has been shown to stabilize U:A base pairs and destabilize U:G wobble pairs (Rodriguez-Hernandez et al., J. Mol. Biol. 2013; 425:3888-3906). Methylation of 2′-hydroxyl groups is one of the most common posttranscriptional modifications of naturally occurring stable RNA molecules (Satoh et al., RNA 2000. 6: 680-686). For example, methylation of tRNA at the 2′-OH position of the ribose sugar is generally thought to increase the stability of tRNA via mechanisms that protect against spontaneous hydrolysis or nuclease digestion (e.g., in non-helical regions) and reinforce intra-loop interactions that stabilize the tertiary structure of the molecule (Endres et al., PLoS ONE 15 (2): e0229103).

Any number of nucleotides (e.g., uridine and/or cytidine) in a particular circRNA molecule generated as described herein may be modified (e.g., replaced) with a corresponding number of 2-thiouridine (2ThioU) or 2′-O-methylcytidine (2OMeC). Ideally, at least one nucleotide in the circRNA molecule is replaced with a 2ThioU or a 2OMeC. In some embodiments, at least 1% (e.g., 1%, 2%, 3%, 4%, 5%, 6%, 7%, 8%, 9% or more) of the nucleotides in the recombinant circular RNA molecule are replaced with 2ThioU or a 2OMeC. In other embodiments, at least 10% (e.g., 10%, 11%, 12%, 13%, 14%, 15%, 20%, 30%, 40%, 50%, 60%, 70%, 80%, 90%, or more) of the nucleotides in the recombinant circular RNA molecule are replaced with 2ThioU or 2OMeC. For example, the recombinant circRNA molecule comprises about 2% to about 5% (e.g., 2.5%, 3%, 3.5%, 4%, or 4.5%) 2-thiouridine or 2-O-methylcytidine. In some embodiments, the recombinant circRNA molecule comprises about 2.5% 2ThioU or 2OMeC. In other embodiments, all (i.e., 100%) of the uridine nucleotides in the recombinant circular RNA molecule may be replaced with 2ThioU, or all (i.e., 100%) of the cytidine nucleotides in the recombinant circRNA molecule may be replaced with 2OMeC. It will be appreciated that the number of 2ThioU or 2OMeC modifications introduced into a recombinant circular RNA molecule will depend upon the particular use of the circRNA.

In some embodiments, a DNA sequence encoding a circular RNA molecule comprises sequences that encode at least two introns and at least one exon. The term “exon,” as used herein, refers to a nucleic acid sequence present in a gene which is represented in the mature form of an RNA molecule after excision of introns during transcription. Exons may be translated into protein (e.g., in the case of messenger RNA (mRNA)). The term “intron,” as used herein, refers to a nucleic acid sequence present in a given gene which is removed by RNA splicing during maturation of the final RNA product. Introns are generally found between exons. During transcription, introns are removed from precursor messenger RNA (pre-mRNA), and exons are joined via RNA splicing. In some embodiments, the recombinant circular RNA molecule comprises a nucleic acid sequence which includes one or more exons and one or more introns.

Accordingly, circular RNAs can be generated using either an endogenous or exogenous intron, as described in WO 2017/222911. As used herein, the term “endogenous intron” means an intron sequence that is native to the host cell in which the circRNA is produced. For example, a human intron is an endogenous intron when the circRNA is expressed in a human cell. An “exogenous intron” means an intron that is heterologous to the host cell in which the circRNA is generated. For example, a bacterial intron would be an exogenous intron when the circRNA is expressed in a human cell. Numerous intron sequences from a wide variety of organisms and viruses are known and include sequences derived from genes encoding proteins, ribosomal RNA (rRNA), or transfer RNA (tRNA). Representative intron sequences are available in various databases, including the Group I Intron Sequence and Structure Database (rna.whu.edu.cn/gissd/), the Database for Bacterial Group II Introns (webapps2.ucalgary.ca/-groupii/index.html), the Database for Mobile Group II Introns (fp.ucalgary.ca/group2introns), the Yeast Intron DataBase (emblS16 heidelberg.de/ExternalInfo/seraphin/yidb.html), the Ares Lab Yeast Intron Database (compbio.soe.ucsc.edu/yeast_introns.html), the U12 Intron Database (genome.crg.es/cgibin/ul2db/ul2db.cgi), and the Exon-Intron Database (bpg.utoledo.edu/-afedorov/lab/eid.html).

In some embodiments, a nucleic acid (e.g., a DNA) encoding a circular RNA molecule comprises a self-splicing group I intron. Group I introns are a distinct class of RNA self-splicing introns which catalyze their own excision from mRNA, tRNA, and rRNA precursors in a wide range of organisms. All known group I introns present in eukaryote nuclei interrupt functional ribosomal RNA genes located in ribosomal DNA loci. Nuclear group I introns appear widespread among eukaryotic microorganisms, and the plasmodial slime molds (myxomycetes) contain an abundance of self-splicing introns. The self-splicing group I intron included in the DNA encoding the circular RNA molecule may be obtained or derived from any organism, such as, for example, bacteria, bacteriophages, and eukaryotic viruses. Self-splicing group I introns also may be found in certain cellular organelles, such as mitochondria and chloroplasts, and such self-splicing introns may be incorporated into the nucleic acid encoding a circular RNA molecule.

In some embodiments, a nucleic acid encoding a recombinant circular RNA molecule comprises a self-splicing group I intron of the phage T4 thymidylate synthase (td) gene. The group I intron of phage T4 thymidylate synthase (td) gene is well characterized to circularize while the exons linearly splice together (Chandry and Belfort, Genes Dev., 1: 1028-1037 (1987); Ford and Ares, Proc. Natd. Acad. Sci. USA, 91: 3117-3121 (1994); and Perriman and Ares, RNA, 4: 1047-1054 (1998)). When the td intron order is permuted (i.e., 5′ half placed at the 3′ position and vice versa) flanking any exon sequence, the exon is circularized via two autocatalytic transesterification reactions (Ford and Ares, supra; Puttaraju and Been, Nucleic Acids Symp. Ser., 33: 49-51 (1995)).

In some embodiments, a nucleic acid (e.g., a DNA) encoding the recombinant circular RNA molecule comprises a ZKSCAN1 intron. The ZKSCAN1 intron is described in, for example, Yao, Z., et al., Mol. Oncol. (2017) 11(4):422-437. In some embodiments, a nucleic acid encoding the recombinant circular RNA molecule comprises a miniZKSCAN1 intron.

The recombinant circular RNA molecule may be of any length or size. For example, the recombinant circular RNA molecule may comprise between about 200 nucleotides and about 10,000 nucleotides (e.g., about 300, about 400, about 500, about 600, about 700, about 800, about 900, about 1,000, about 2,000, about 3,000, about 4,000, about 5,000, about 6,000, about 7,000, about 8,000, or about 9,000 nucleotides, or a range defined by any two of the foregoing values). In some embodiments, the recombinant circular RNA molecule comprises between about 500 and about 6,000 nucleotides (about 550, about 650, about 750, about 850, about 950, about 1,100, about 1,200, about 1,300, about 1,400, about 1,500, about 1,600, about 1,700, about 1,800, about 1,900, about 2,100, about 2,200, about 2,300, about 2,400, about 2,500, about 2,600, about 2,700, about 2,800, about 2,900, about 3,100, about 3,300, about 3,500, about 3,700, about 3,800, about 3,900, about 4,100, about 4,300, about 4,500, about 4,700, about 4,900, about 5,100, about 5,300, about 5,500, about 5,700, or about 5,900 nucleotides, or a range defined by any two of the foregoing values). In one embodiment, the recombinant circular RNA molecule comprises about 1,500 nucleotides.

In some embodiments, a recombinant circular RNA molecule comprises an internal ribosome entry site (IRES) sequence operably linked to a protein-coding sequence; wherein the IRES sequence is a viral sequence; and wherein the protein-coding sequence encodes a non-viral protein.

In some embodiments, a recombinant circular RNA molecule comprises a protein-coding nucleic acid sequence region and an internal ribosome entry site (IRES) sequence region operably linked to the protein-coding nucleic acid sequence region, wherein the IRES comprises: at least one sequence region having secondary structure element; and a sequence region that is complementary to an 18S ribosomal RNA (rRNA); wherein the IRES has a minimum free energy (MFE) of less than −18.9 kJ/mol and a melting temperature of at least 35.0° C. In some embodiments, the IRES sequence is linked to the protein-coding nucleic acid sequence region in a non-native configuration.

The disclosure also provides a recombinant circular RNA molecule comprising a protein-coding nucleic acid sequence region and an internal ribosome entry site (IRES) sequence region operably linked to the protein-coding nucleic acid sequence; wherein the IRES is encoded by any one of the nucleic acid sequences listed in SEQ ID NOs: 138-17338, or a nucleic acid sequence that has at least 90% or at least 95% identity or homology thereto. In some embodiments, the IRES sequence is linked to the protein-coding nucleic acid sequence region in a non-native configuration.

### Internal Ribosome Entry Sequences

The recombinant circular RNAs described herein comprise an internal ribosome entry site (IRES). These IRES sequences may be operably linked to a protein-coding sequence of the circRNA. Inclusion of an IRES permits the translation of one or more open reading frames from a circular RNA. The IRES attracts a eukaryotic ribosomal translation initiation complex and promotes translation initiation.

Provided herein are various IRES sequences which, when present in a circRNA, drive translation of a protein encoded by the circRNA. In some embodiments, the IRES of a circRNA may be operably linked to a protein-coding nucleic acid sequence. In some embodiments, the IRES of a circRNA is operably linked to a protein-coding nucleic acid sequence in a non-native configuration. In some embodiments, the IRES is a human TRES. In some embodiments, the IRES is a viral IRES. In some embodiments, the IRES is a type 1 IRES.

As used herein, the term “non-native configuration” refers to a linkage between an IRES and a protein-coding nucleic acid that does not occur in a naturally occurring circRNA molecule. For example, a viral IRES may be operably linked to a protein-coding nucleic acid sequence in a circular RNA, or an IRES that is not found in naturally occurring circRNA molecules may be operably linked to a protein-coding nucleic acid sequence in a circRNA. In some embodiments, an IRES that is found in naturally occurring circRNA molecules operably linked to a certain protein-coding nucleic acid is operably linked to a different protein-coding nucleic acid (i.e., a nucleic acid to which the IRES is not operably linked in any naturally-occurring circRNA). In some embodiments, an IRES that is found in naturally occurring linear mRNAs is operably linked to a protein coding sequence in a circular RNA.

A number of linear IRES sequences are known and may be included in a recombinant circular RNA molecule as described herein. For example, linear IRES sequences may be derived from a wide variety of viruses, such as from leader sequences of picornaviruses (e.g., encephalomyocarditis virus (EMCV) UTR) (Jang et al., J. Virol., 63: 1651-1660 (1989)), the polio leader sequence, the hepatitis A virus leader, the hepatitis C virus IRES, human rhinovirus type 2 IRES (Dobrikova et al., Proc. Natl. Acad. Sci., 100(25): 15125-15130 (2003)), an IRES element from the foot and mouth disease virus (Ramesh et al., Nucl. Acid Res., 24: 2697-2700 (1996)), and a giardiavirus IRES (Garlapati et al., J. Biol. Chem., 279(5): 3389-3397 (2004)). A variety of nonviral IRES sequences also can be included in a circular RNA molecule, including but not limited to, IRES sequences from yeast, the human angiotensin II type 1 receptor IRES (Martin et al., Mol. Cell Endocrinol., 212: 51-61 (2003)), fibroblast growth factor IRESs (e.g., FGF-1 IRES and FGF-2 IRES, Martineau et al., Mol. Cell. Biol., 24(17): 7622-7635 (2004)), vascular endothelial growth factor IRES (Baranick et al., Proc. Natl. Acad. Sci. U.S.A., 105(12): 4733-4738 (2008); Stein et al., Mol. Cell. Biol., 18(6): 3112-3119 (1998); Bert et al., RNA, 12(6): 1074-1083(2006)), and insulin-like growth factor 2 IRES (Pedersen et al., Biochem. J., 363(Pt 1): 37-44 (2002)).

IRES sequences and vectors encoding IRES elements are commercially available from a variety of sources, such as, for example, Clontech (Mountain View, CA), Invivogen (San Diego, CA), Addgene (Cambridge, MA) and GeneCopoeia (Rockville, MD), and IRESite: The database of experimentally verified IRES structures (iresite.org). Notably, these databases focus on activity of IRES sequences in mRNA (i.e., linear RNAs), and do not focus on circRNA IRES activity profiles.

### Viral IRES Sequences

In some embodiments, the circRNAs described herein comprise viral IRES sequence. The viral IRES sequence may be operably linked to a protein-coding sequence in a non-native configuration. For example, the viral IRES sequence may be operably linked to a sequence that encodes a non-viral protein. In some embodiments, the protein coding sequence encodes an animal protein, a plant protein, a bacterial protein, a fungal protein, or an artificial protein. In some embodiments, the protein coding sequence encodes a mammalian protein, such as a human protein. In some embodiments, the viral IRES sequence, when placed into a circular RNA, drives potent translation of a protein encoded by the circular RNA.

Table 7 below provides a non-limiting list of viral IRES that may be used in a circRNA to drive expression of a protein encoded by the circular RNA. Also provided in Table 7 are GenBank Accession Nos. for the genomic sequences from which the viral IRES were identified. Sequences encoding the viral IRES are provided in the SEQUENCE APPENDIX.

TABLE 7
Illustrative viral IRES sequences

| Viral IRES | GenBank Accession No. |
|---|---|
| iPV1 | NC_002058 |
| iPV2 | D00625 |
| iPV3 | AJ293918 |
| iHRV1 | NC_038311 |
| iHRV2 | X02316 |
| iHRV-B3 | NC_038312 |
| iCVA1 | AF499635 |
| iCVA16 | KC117318 |
| iCVB2 | AF081485 |
| iCVB3 | NC_038307 |
| iEV-A71 | KT354875 |
| iEV-A120 | LK021688 |
| iEV71 | HM003207 |
| iEV-B107 | AB426609 |
| iHRV-A9 | FJ445177 |
| iHRV-A21 | FJ445121 |
| iHRV-A57 | JN614995 |
| iHRV-A89 | NC_001617 |
| iHRV-A100 | FJ445175 |
| iHRV-B4 | DQ473490 |
| iHRV-B17 | EF173420 |
| iHRV-B26 | FJ445124 |
| iHRV-B37 | EF173423 |
| iHRV-B92 | FJ445169 |
| iHRV-C3 | EF186077 |
| iHRV-C11 | EU840952 |
| iHRV-C54 | KP282614 |
| iCVB1 | M16560 |
| iCVB5 | AF114383 |
| iEchoV1 | AF029859 |
| iEchoV-E11 | X80059 |
| iEchoV-E15 | AY302541 |
| iCVA20 | AF499642 |
| iEV-D94 | DQ916376 |
| iSimianEV-A | NC_003988 |
| iSimianA5 | AF326751 |
| iSimianV4 | NC_038309 |
| iSwineVesciular | MK038999 |
| iHRV-B3_SC2220 | KY684757 |
| iHRV-B5 | FJ445112 |
| iHRV-B6 | DQ473486 |
| iHRV-B14 | MN306034 |
| iHRV-B27 | FJ445186 |
| iHRV-B35 | DQ473487 |
| iHRV-B42 | FJ445130 |
| iHRV-B48 | DQ473488 |
| iHRV-B52 | FJ445188 |
| iHRV-B69 | FJ445151 |
| iHRV-B70 | DQ473489 |
| iHRV-B72 | FJ445153 |
| iHRV-B79 | FJ445155 |
| iHRV-B83 | MN306025 |
| iHRV-B84 | FJ445162 |
| iHRV-B86 | FJ445164 |
| iHRV-B91 | FJ445168 |
| iHRV-B93 | EF173425 |
| iHRV-B97 | FJ445172 |
| iHRV-B99 | FJ445174 |
| iHRVB_SC0739 | MN306037 |
| iHRVB_G001-19 | MN212904 |
| iHRVB_R93 | KF958309 |
| iHRVB_BCH214 | GU568096 |
| iHRVB_3039 | KF958308 |
| iEV-B69 | AY302560 |
| iEV-B73 | AF241359 |
| iEV-B74 | AY556057 |
| iEV-B75 | AY556070 |
| iEV-B77 | AJ493062 |
| iEV-B79 | AB426610 |
| iEV-B80 | AY843298 |
| iEV-B81 | AY843299 |
| iEV-B82 | AY843300 |
| iEV-B83 | AY843301 |
| iEV-B84 | DQ902712 |
| iEV-B85 | AY843303 |
| iEV-B86 | AY843304 |
| iEV-B87 | KC292019 |
| iEV-B88 | AY843306 |
| iEV-B93 | MN580134 |
| iEV-B97 | AY843307 |
| iEV-B98 | AB426608 |
| iEV-B100 | DQ902713 |
| iEV-B101 | AY843308 |
| iEV-B106 | KF990476 |
| iEV-B107_TN94-0349 | AB426609 |
| iEV-B111 | KF312882 |
| iEV-B113 | KC344833 |
| iEV-D68 | NC_038308 |
| iEV-D70 | NC_001430 |
| iEV-D94 | DQ916376 |
| iEV-D111 | MK032895 |
| iHCosV-B1 | NC_012801 |
| iEcho-E7 | LS451295 |
| iEcho-E9 | AF524866 |
| iPV3_PAK1019536 | KY941934.1 |
| iPV3_NIE1219535 | KY941933.1 |
| iPV3_SWI10947 | FJ914252.2 |
| iPV3_FIN84 | FJ842158.1 |

In some embodiments, a circRNA comprises any one of the IRES in Table 7, or a fragment or derivative thereof. In some embodiments, a circRNA comprises an IRES encoded by any one of SEQ ID NO: 101-125, or a fragment or derivative thereof.

In some embodiments, the IRES is a Type 1 IRES. Type I IRES elements occur in the RNA genome of enterovirus species, including poliovirus (PV), coxsackievirus B3 (CVB3), enterovirus 71 (EV71), and human rhinovirus (HRV). In some embodiments, the IRES is an enterovirus IRES. In some embodiments, the IRES is an HRV IRES.

In some embodiments, a circRNA comprises any one of the following IRES: iCVA20; iEchoV-E11, iSimianEV-A, iCovid19, iHRV-A57, iEchoVl1, iCrPV, iHRV-A89, iHRV-B26, iBEV, iEchoVl, iHRV-A21, iPV1, iCVB3, iEMCV, iHCV, iCVB5, iSwineVesicular, iHRV-A2, iHRV-C3, iHRV-C11, iCVB1, iPV2, iHRV-B17, iEchoV-E15, iEV71, iHRV-A9, iSiminanV4, iEV-D94, iSimianA5, iPV3, iHRV-C54, iHRV-A100, iHRV-B37, iHRV-B4, iHRV-B92, iHRV-B3, iHRV-A1, iEV107, or a fragment or derivative thereof.

In some embodiments, a circRNA comprises any one of the following IRES: iEMCV, iHCV, iCVB5, iSwineVesicular, iHRV-A2, iHRV-C3, iHRV-C11, iCVB1, iPV2, iHRV-B17, iEchoV-E15, iEV71, iHRV-A9, iSiminanV4, iEV-D94, iSimianA5, iPV3, iHRV-C54, iHRV-A100, iHRV-B37, iHRV-B4, iHRV-B92, iHRV-B3, iHRV-A1, iEV107, or a fragment or derivative thereof.

In some embodiments, a circRNA comprises any of the following IRES: iEV-B79, iEV-B77, iPV3 SWI10947, iHRV-B26, iHRV-B37, iHRV-A89, iEV-B86, iEV-B1113, iEV-B87, iHRVA021, iEV-B88, iHRV-C11, iEV-B93, iEVD70, iEV-B111, iHRV-B92, iEV-B69, iEV-B73, iEV-B107, iEV107, iHRV-C54, iEV-B100, iHRVB_BCH214, iEV-B98, iPV3_NIE21219535, iEV-D111, iEcho-E9, iEV-B82, iEV-D94, iEV-B75, iEV97, iEV-B84, iHRV-C3, iHRV-A1, iEcho-E7, iEV-B81, iPV3_PAK1019536, iHRV-A9, iEV-B106, iHRV-A100, iPV3 FIN84, iEV-B85, iHRV-B86, iEV-B101, iHRV-B3, iHRV-B17, iHRVB_G001-10, iHRV-B70, iEV-B74, iEV-B80, iCVB3, iEV-B83, iHRV-A57, iHRV-B35, iHRV-B4, iEV-D68, iHRVB_R93, iHRV-B5, iHRVB-B52, iHRVB-B93, iHRV-B84, iHRV-B83_SC2220, iHRV-B72, iHRV-B69, iHRVB_SC0739, iHRV-B91, iHRV-B42, iHRV-B6, iHRV-B83, iHRV-B48, iHRV-B99, iHRV-B79, iHRV-B97, iHRV-B27, iHRVB 3039, iHRVB-B14, iCosV-B1.

In some embodiments, a circRNA comprises any of the following IRES: iEV-B83, iHRV-A57, iHRV-B35, iHRV-B4, iEV-D68, iHRVB_R93, iHRV-B5, iHRVB-B52, iHRVB-B93, iHRV-B84, iHRV-B83_SC2220, iHRV-B72, iHRV-B69, iHRVB_SC0739, iHRV-B91, iHRV-B42, iHRV-B6, iHRV-B83, iHRV-B48, iHRV-B99, iHRV-B79, iHRV-B97, iHRV-B27, iHRVB_3039, iHRVB-B14, iCosV-B1, or a fragment or derivative thereof.

In some embodiments, a circRNA comprises the iCVB3 IRES. In some embodiments, a circRNA comprises a fragment or derivative of the iCVB3 IRES.

In some embodiments, a circRNA comprises the iHRV-B3 IRES. In some embodiments, a circRNA comprises a fragment or derivative of the iHRV-B3 IRES.

### Synthetic IRES

In some embodiments, a circRNA comprises a synthetic IRES. A “synthetic IRES” is an IRES that is modified relative to a wildtype IRES in order to modulate its structure and/or activity. For example, in some embodiments, an IRES that is modified to incorporate an aptamer sequence is a synthetic IRES.

In some embodiments, a synthetic IRES comprises an aptamer. In some embodiments, a synthetic IRES comprises a first aptamer and a second aptamer. In some embodiments, a synthetic comprises two, three, four, five, six, seven, eight, nine, ten, or more aptamers.

In some embodiments, the aptamer is a wildtype aptamer. In some embodiments, the aptamer is a fragment of a wildtype aptamer. In some embodiments, the aptamer is an aptamer that was designed to bind DNA or RNA. Synthetic aptamers can be created that bind a specific DNA or RNA sequence by evolution through one or more rounds of evolution using, for example, SELEX technology.

In some embodiments, the aptamer is a modified version of a known aptamer (e.g., a mutant aptamer). In some embodiments, the aptamer is modified to have an extended stem region. For example, the length of the stem region may be extended by about 10% to about 25%, about 25% to about 50%, about 50% to about 75%, about 75% to about 100%, about 125%, about 150%, about 175%, about 200% or more. In some embodiments, the length of the stem region is extended by about 1, about 2, about 3, about 4, about 5, about 6, about 7, about 8, about 9, about 10 base pairs. As will be understood by those of skill in the art, extension of a stem region by 1 base pair comprises adding 2 nucleotides to the aptamer sequence. Accordingly, an aptamer which comprises a stem region extended by 3 base pairs have a nucleotide sequence that is 6 nucleotides longer than the same aptamer in which the stem region is not extended.

The aptamer may be inserted into the IRES sequence in any location which is permissive to such changes. In some embodiments, the aptamer is positioned within the secondary structure of the IRES so that is spatially proximal to portion of the IRES responsible for translation initiation. In some embodiments, the aptamer is located in a position where it can bind to one or more translation initiation factors, such as eIF4G. In some embodiments, the aptamer does not interrupt the native eIF4G binding site of the IRES. In some embodiments, the IRES does not interrupt a native GRNA tetraloop within the IRES.

In some embodiments, the aptamer is an eIF4G-binding aptamer, such as any one of the aptamers listed in Table 6. In some embodiments, the aptamer is a fragment or derivative of any of the aptamers listed in Table 6. In some embodiments, the eIF4G-binding aptamer comprises or is encoded by the sequence of SEQ ID NO: 99. In some embodiments, the eIF4G-binding aptamer comprises the sequence of SEQ ID NO. 134.

TABLE 6
eIF4G-Binding Aptamers

| Aptamer | | | SEQ ID |
|---|---|---|---|
| ID | DNA Sequence | RNA Sequence | NOs: |
| A | TAA TAC GAC TCA CTA | UAAUACGACUCACUAUAGG | 126, 130 |
| | TAG GG ACTC ACT ATT TGT | GACUCACUAUUUGUUUUCG | |
| | TTT CGC GCC CAG TTG | CGCCCAGUUGCAAAAAGUG | |
| | CAA AAA GTG TCG CCG | UCGCCGCAUCUAGAGGGCC | |
| | CAT CTA GAG GGC C | | |
| B | TAA TAC GAC TCA CTA | UAAUACGACUCACUAUAGG | 127, 131 |
| | TAG GGT CCA G?C CCC | GUCCAGACCCCAACAGACU | |
| | AAC AGA CTC CAT AAC | CCAUAACUAAUAUGUCGCA | |
| | TAA TAT GTC GCA AAA | AAACCGCAUCUAGAGGGCC | |
| | CCG CAT CTA GAG GGC C | | |
| C | TAA TAC GAC TCA CTA | UAAUACGACUCACUAUAGG | 128, 132 |
| | TAG GGT CCG TAG AAA | GUCCGUAGAAACGCGUUAA | |
| | CGC GTT AAG GTG AAA | GGUGAAAGUUUGAGGGCUC | |
| | GTT TGA GGG CTC CTC | CUCACCGCAUCUAGAGGGC | |
| | ACC GCA TCT AGA GGG CC | C | |
| D | TAA TAC GAC TCA CTA | UAAUACGACUCACUAUAGG | 129, 133 |
| | TAG GGT CCG CGG CGC | GUCCGCGGCGCCAUCUCAU | |
| | CAT CTC ATG TTT AGT TGT | GUUUAGUUGUCCUAUGUCG | |
| | CCT ATG TCG AGC CCG | AGCCCGCAUCUAGAGGGCC | |
| | CAT CTA GAG GGC C | | |
| E | ACTCACTATTTGTTTTCGCG | ACUCACUAUUUGUUUUCGC | 99, 134 |
| | CCCAGTTGCAAAAAGTGTC | GCCCAGUUGCAAAAAGUGU | |
| | G | CG | |

In some embodiments, the IRES is a type I TRES. In some embodiments, the IRES is an enterovirus IRES. In some embodiments, the TRES is an HIRV IREs.

SEQ ID NO: 101-125 shown in the SEQUENCE APPENDIX provide illustrative IRES sequences, wherein the TRES sequences comprise an aptamer. The aptamer insertion is shown in capital letters.

In some embodiments, a synthetic TRES sequence comprises a modified iCVB3 IRES. In some embodiments, the modified iCVB3 TRES comprises an aptamer inserted in domain I, II, III, IV, V, VI, or VII thereof. In some embodiments the modified iCVB3 TRES comprises an aptamer inserted in domain I, II, III, IV, V, VI, or VII thereof, in a location that minimally disrupts the native RNA structure. In some embodiments, the modified iCVB3 IRES comprises an aptamer inserted in domain IV thereof. In some embodiments, the aptamer is modified to have an extended stem region. The stem region may be extended, for example, by 1, 2, 3, 4, 5, 6, or more base pairs. In some embodiments, the aptamer is positioned within the secondary structure of the IRES so that is spatially proximal to portion of the IRES responsible for translation initiation. In some embodiments, the aptamer does not interrupt the native eIF4G binding site of the IRES and/or does not interrupt a native GRNA tetraloop within the IRES.

In some embodiments, a synthetic IRES sequence comprises a modified iHRV-B3 IRES. In some embodiments, the modified iHRV-B3 IRES comprises an aptamer inserted in domain I, II, III, IV, V, VI, or VII thereof. In some embodiments, the modified iHRV-B3 IRES comprises an aptamer inserted in domain IV thereof. In some embodiments, the aptamer is modified to have an extended stem region. The stem region may be extended, for example, by 1, 2, 3, 4, 5, 6, or more base pairs. In some embodiments, the aptamer is positioned within the secondary structure of the IRES so that is spatially proximal to portion of the IRES responsible for translation initiation. In some embodiments, the aptamer does not interrupt the native eIF4G binding site of the IRES and/or does not interrupt a native GRNA tetraloop within the IRES.

### IRES Elements and Features

In some embodiments, a circRNA comprises an IRES, such as a synthetic or viral IRES, that comprises one or more of the IRES elements or features described below.

In some embodiments, a circRNA comprises an IRES that comprises at least one RNA secondary structure element. Intramolecular RNA base pairing is often the basis of RNA secondary structure and in some circumstances be a critical determinant of overall macromolecular folding. In conjunction with cofactors and RNA binding proteins (RBPs), secondary structure elements can form higher order tertiary structures and thereby confer catalytic, regulatory, and scaffolding functions to RNA. Thus, the IRES may comprise any RNA secondary structure element that imparts such structural or functional determinants.

In some embodiments, the RNA secondary structure may be formed from the nucleotides at about position 40 to about position 60 of the IRES, relative to the 5′ end thereof. The most common RNA secondary structures are helices, loops, bulges, and junctions, with stem-loops or hairpin loops being the most common element of RNA secondary structure. A stem-loop is formed when the RNA chains fold back on themselves to form a double helical tract called the stem, with the unpaired nucleotides forming a single-stranded region called the loop. Bulges and internal loops are formed by separation of the double helical tract on either one strand (bulge) or on both strands (internal loops) by unpaired nucleotides. A tetraloop is a four-base pairs hairpin RNA structure. There are three common families of tetraloop in ribosomal RNA: UNCG (SEQ ID NO: 135), GNRA (SEQ ID NO: 136), and CUUG (SEQ ID NO: 137) (N is one of the four nucleotides and R is a purine). Pseudoknots are formed when nucleotides from the hairpin loop pair with a single stranded region outside of the hairpin to form a helical segment. RNA secondary structure is further described in, e.g., Vandivier et al., Annu Rev Plant Biol., 67: 463-488 (2016); and Tinoco and Bustamante, supra). In some embodiments, the IRES of the recombinant circRNA molecule comprises at least one stem-loop structure. The at least one RNA secondary structure element may be located at any position of the IRES, so long as translation is efficiently initiated from the IRES. In some embodiments, the stem portion of the stem-loop may comprise from 3-7 base pairs, 4, 5, 6, 7, 8, 9, 10, 11 or 12 base pairs or more. The loop portion of the stem-loop may comprise from 3-12 nucleotides, including 4, 5, 6, 7, 8, 9, 10, 11, 12 or more nucleotides. The stem-loop structure may also have on either side of the stem one or more bulges (mismatches). In some embodiments, the RNA secondary structure element is formed from the nucleotides at about position 40 to about position 60 of the IRES, wherein the first nucleic acid at the 5′ end of the IRES is considered to be position 1. In some embodiments, the sequence that is complementary to an 18S rRNA is located 5′ to the at least one RNA secondary structure element (i.e., in the range of about position 1 to about position 40 of the IRES). In some embodiments, the sequence that is complementary to an 18S rRNA is located 3′ to the a least one RNA secondary structure element (i.e., in the range of about position 61 to the end of the IRES). Sequences encoding exemplary secondary structure-forming RNA sequences that may be included in the IRES described herein are set forth in SEQ ID NOs: 17339-29113.

In some embodiments, the at least one RNA secondary structure element of the IRES is a stem-loop. In some embodiments, the at least one RNA secondary structure element is encoded by any one of the nucleic acid sequences listed in SEQ ID NOs: 17339-29113. In some embodiments, the at least one RNA secondary structure element is encoded by a nucleic acid sequence having at least 80%, at least 85%, at least 90%, at least 91%, at least 92%, at least 93%, at least 94%, at least 95%, at least 96%, at least 97%, at least 98%, or at least 99% sequence identity relative to any one of the nucleic acid sequences listed in SEQ ID NOs: 17339-29113. In some embodiments, the at least one RNA secondary structure element is encoded by a nucleic acid sequence having at least 1, at least 2, at least 3, at least 4, at least 5, at least 6, at least 7, at least 8, at least 9 at least 10, or more nucleotide substitutions relative to any one of the nucleic acid sequences listed in SEQ ID NOs: 17339-29113.

RNA secondary structure typically can be predicted from experimental thermodynamic data coupled with chemical mapping, nuclear magnetic resonance (NMR) spectroscopy, and/or sequence comparison. In some embodiments, the RNA secondary structure is predicted by a machine-learning/deep-learning algorithm (e.g., CNN) (See, Zhao, Q., et al., “Review of Machine-Learning Methods for RNA Secondary Structure Prediction,” Sep. 1, 2020 (available on the world wide web at: arxiv.org/abs/2009.08868). A variety of algorithms and software packages for RNA secondary structure prediction and analysis are known in the art and can be used in the context of the present disclosure (see, e.g., Hofacker I.L. (2014) Energy-Directed RNA Structure Prediction. In: Gorodkin J., Ruzzo W. (eds) RNA Sequence, Structure, and Function: Computational and Bioinformatic Methods. Methods in Molecular Biology (Methods and Protocols), vol 1097. Humana Press, Totowa, NJ; Mathews et al., supra; Mathews, et al. “RNA secondary structure prediction,” Current Protocols in Nucleic Acid Chemistry, Chapter 11 (2007): Unit 11.2. doi:10.1002/0471142700.nc1102s28; Lorenz et al., Methods, 103: 86-98 (2016); Mathews et al., Cold Spring Harb PerspectBiol., 2(12): a003665 (2010)).

In some embodiments, the IRES of the recombinant circRNA may comprise a nucleic acid sequence that is complementary to 18S ribosomal RNA (rRNA). Eukaryotic ribosomes, also known as “80S” ribosomes, have two unequal subunits, designated small subunit (40S) (also referred to as “SSU”) and large subunit (60S) (also referred to as “LSU”) according to their sedimentation coefficients. Both subunits contain dozens of ribosomal proteins arranged on a scaffold composed of ribosomal RNA (rRNA). In eukaryotes, eukaryotic 80S ribosomes contain greater than 5500 nucleotides of rRNA: 18S rRNA in the small subunit, and 5S, 5.8S, and 25S rRNA in the large subunit. The small subunit monitors the complementarity between tRNA anticodon and mRNA, while the large subunit catalyzes peptide bond formation. Ribosomes typically contain about 60% rRNA and about 40% protein. Although the primary structure of rRNA sequences can vary across organisms, base-pairing within these sequences commonly forms stem-loop configurations.

In some embodiments, the IRES of the recombinant circRNA may comprise any nucleic acid sequence that is complementary to any eukaryotic 18S rRNA sequence. In some embodiments, the nucleic acid sequence that is complementary to 18S rRNA is encoded by any one of the nucleic acid sequences set forth in Table 3. In some embodiments, the nucleic acid sequence that is complementary to 18S rRNA is encoded by a nucleic acid sequence that has at least 80%, at least 85%, at least 90%, at least 91%, at least 92%, at least 93%, at least 94%, at least 95%, at least 96%, at least 97%, at least 98%, or at least 99% identity or homology to a sequence set forth in Table 3. In some embodiments, the nucleic acid sequence that is complementary to 18S rRNA is encoded by a nucleic acid sequence that has at least 1, at least 2, at least 3, at least 4, at least 5, at least 6, at least 7, at least 8, at least 9, at least 10, or more nucleotide substitutions relative to a nucleic acid sequence set forth in Table 3.

TABLE 3
Illustrative DNA sequences that encode RNA sequences that are
complementary to 18S RNA

| SEQ ID | |
|---|---|
| NO: | Nucleic Acid Sequence |
| 33084 | CACCTACGGAAACCTTGTTACGACTTTTACTTCCTCTAGATAGTCAAGTTC |
| | GACCGTCTTCTCAGCGCTCCGCCAGGGCCGTGGGCCGACCCCGGCGGGGC |
| | CGATCCGAGGGCCTCACTAAACCATCCAATCGGTAGTAGCGACGGGCGGT |
| | GTGTACAAAGGGCAGGGACTTAATCAACGCAAGCTTATGACCCGCACTTA |
| | CTGGGAATTCCTCGTTCATGGGGAATAATTGCAATCCCC |
| 33085 | GGGACGCCGACCGCTCGGGGGTCGCGTAACTAGTTAGCATGCCAGAGTCT |
| | CGTTCGTTATCGGAATTAACCAGACAAATCGCTCCACCAACTAAGAACGG |
| | CCATGCACCACCACCC |
| 33086 | GTTTATGGTCGGAACTACGACGGTATCTGATCGTCTTCGAACCTCCGACTT |
| | TCGTTCTTGATTAATGAAAACATTCTTGGCAAATGCTTTCGCTCTGGTCCGT |
| | CTTGCGCCGGTCCAAGAATTTCACCTCTAGCGGCGCAATACGAATGCCCCC |
| | GGCCGTCCCTCTTAATCATGGCCTCAGTTCCGAAAACCAACAAAATAGAA |
| | CCGCGGTCCTATTCCATTATTCCTAGCTGCGGTATCCAGGCGGCTCGGGCC |
| | TGCTTTGAACACTCTAATTTTTTCAAAGTAAACGCTTCGGG |
| 33087 | AGAGCATCGAGGGGGCGCCGAGAGGCAAGGGGGGGGACGGGCGGTGGC |
| | TCGCCTCGCGGCGGACCGCCCGCCCGCTCCC |
| 33088 | TACGAGCTTT |
| 33089 | ATACGCTATTGGAGCTGGAATTACCGCGGCTGCTGGCACCAGACTTGCCCT |
| | CCAATGGATCCTCGTTAAAGGATTTAAAGTGGACTCATTCCAATTACAGGG |
| | CCTCGAAAGAGTCCTGTA |
| 33090 | GGGGGCGTGCGATCGGCCCGAGGTTATCTAGAGTCACCAAAGCCGCCGGC |
| | GCCCGCCCCCCGGCCGGGGCCGGAGAGGGG |

The most commonly used criterion for RNA secondary structure prediction is the minimum free energy (MFE), since, according to thermodynamics, the MFE structure is not only the most stable, but also the most probable one in thermodynamic equilibrium. The MFE of an RNA or DNA molecule is affected by three properties of nucleotides in the RNA/DNA sequence: number, composition, and arrangement. For example, longer sequences are on average more stable because they can form more stacking and hydrogen bond interactions, guanine-cytosine (GC)-rich RNAs are typically more stable than adenine-uracil (AU)-rich sequences, and nucleotide order influences the folding structure stability because it determines the number and the extension of loops and double-helix conformations. It has been found that mRNAs and microRNA precursors, unlike other non-coding RNAs, have greater negative MFE than expected given their nucleotide numbers and compositions. Thus, free energy also can be employed as a criterion for the identification of functional RNAs.

The IRES of the recombinant circRNA molecule may comprise a minimum free energy (MFE) of less than about −15 kJ/mol (e.g., less than about −16 kJ/mol, less than about −17 kJ/mol, less than about −18.5 kJ/mol, less than about −19 kJ/mol, less than about −18.9 kJ/mol, less than about −20 kJ/mol, less than about −30 kJ/mol). In some embodiments, the MFE is greater than about −90 kJ/mol (e.g., greater than about −85 kJ/mol, greater than about −80 kJ/mol, greater than about −70 kJ/mol, greater than about −60 kJ/mol, greater than about −50 kJ/mol, greater than about −40 kJ/mol). In some embodiments, the IRES has a has a minimum free energy (MFE) of about −18.9 kJ/mol or less. In some embodiments, the IRES has a MFE in the range of about −15.9 kJ/mol to about −79.9 kJ/mol. In some embodiments, the IRES may comprise a MFE in the range of about −12.55 kJ/mol to about −100.15 kJ/mol. In some embodiments, the IRES is a viral IRES and has an MFE in the range of about −15.9 kJ/mol to about −79.9 kJ/mol. In some embodiments, the IRES is a human IRES and has a MFE in the range of about −12.55 kJ/mol to about −100.15 kJ/mol.

In some embodiments, the at least one secondary structure element of an IRES of may comprise a minimum free energy (MFE) of less than about −0.4 kJ/mol, less than about −0.5 kJ/mol, less than about −0.6 kJ/mol, less than about −0.7 kJ/mol, less than about −0.8 kJ/mol, less than about −0.9 kJ/mol, or less than about −1.0 kJ/mol. In some embodiments, the at least one secondary structure element of the IRES may comprise a MFE of less than about −0.7 kJ/mol.

In some embodiments, the RNA sequence comprising the nucleotides at about position 40 to about position 60 of an IRES of a circRNA described herein may comprise a minimum free energy (MFE) of less than about −0.4 kJ/mol, less than about −0.5 kJ/mol, less than about −0.6 kJ/mol, less than about −0.7 kJ/mol, less than about −0.8 kJ/mol, less than about −0.9 kJ/mol, or less than about −1.0 kJ/mol. In some embodiments, the RNA sequence comprising the nucleotides at about position 40 to about position 60 of the IRES may comprise an MFE of less than about −0.7 kJ/mol.

As discussed, above, the minimum free energy of a particular RNA (e.g., an RNA produced from a DNA sequence) may be determined using a variety of computational methods and algorithms. The most commonly used software programs, employed to predict the secondary RNA or DNA structures by MFE algorithms, make use of the so-called nearest-neighbor energy model. This model uses free energy rules based on empirical thermodynamic parameters (Mathews et al., J Mol Biol, 288: 911-940 (1999); and Mathews et al., Proc Natl Acad Sci USA, 101: 7287-7292 (2004)) and computes the overall stability of an RNA or DNA structure by adding independent contributions of local free energy interactions due to adjacent base pairs and loop regions. In sequences with homogeneous nucleotide arrangements and compositions, the additive and independent nature of the local free energy contributions suggests a linear relationship between computed MFE and sequence length (Trotta, E., PLoS One, 9(11): e113380 (2014)). Algorithms for determining MFE are further described in, e.g., Hajiaghayi et al., BMC Bioinformatics, 13: 22 (2012); Mathews, D. H., Bioinformatics, Volume 21, Issue 10: 2246-2253 (2005); and Doshi et al., BMC Bioinformatics, 5: 105 (2004) doi 10.1186/1471-2105-5-105).

One of ordinary skill in the art will appreciate that the melting temperature (Tm) of a particular circRNA molecule may also be indicative of stability. Indeed, RNA sequences with high Tₘ generally contain thermo-stable functionally important RNA structures (see, e.g., Nucleic Acids Res., 45(10): 6109-6118 (2017)). Thus, in some embodiments, the IRES of the recombinant circRNA molecule has a melting temperature of at least 35.0° C. In some embodiments, the IRES of the recombinant circRNA molecule has a melting temperature of at least 35.0° C., but not more than about 85° C. In some embodiments, in some embodiments, the RNA secondary structure has a melting temperature of at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C. or greater. In some embodiments, the melting temperature is not more than about 85° C., not more than about 75° C., not more than about 70° C., not more than about 65° C., not more than about 60° C., not more than about 55° C., not more than about 50° C. or less.

The melting temperature of a particular nucleic acid molecule can be determined using thermodynamic analyses and algorithms described herein and known in the art (see, e.g., Kibbe W. A., Nucleic Acids Res., 35(Web Server issue): W43-W46 (2007). doi:10.1093/nar/gkm234; and Dumousseau et al., BMC Bioinformatics, 13: 101 (2012). doi.org/10.1186/1471-2105-13-101).

In some embodiments, the IRES comprises at least one RNA secondary structure element; and a nucleic acid sequence that is complementary to an 18S ribosomal RNA (rRNA); wherein the IRES has a minimum free energy (MFE) of -18.9 kJ/mol or less and a melting temperature of at least 35.0° C. In some embodiments, the RNA secondary structure element of the IRES has a has a minimum free energy (MFE) of less than −18.9 kJ/mol, and is formed from the nucleotides at about position 40 to about position 60 of the IRES, wherein the first nucleic acid at the 5′ end of the IRES is considered to be position 1. In some embodiments, the RNA secondary structure element has a melting temperature of at least 35.0° C., and is formed from the nucleotides at about position 40 to about position 60 of the IRES, wherein the first nucleic acid at the 5′ end of the IRES is considered to be position 1.

Because circRNA molecules are often generated from linear RNAs by back-splicing of a downstream 5′ splice site (splice donor) to an upstream 3′ splice site (splice acceptor), the recombinant circular RNA molecule may further comprise a back-splice junction. In some embodiments, the IRES may be located within about 100 to about 200 nucleotides of the back-splice junction. In addition, it has been observed that regions of RNA with higher G-C content have more stable secondary structures than RNA strands with lower G-C content. Thus, in some embodiments, the IRES of the recombinant circRNA molecule may further comprise a minimum level of G-C base pairs. For example, the non-native IRES of the recombinant circRNA molecule may comprise a G-C content of at least 25% (e.g., at least 30%, at least 35%, at least 40%, at least 45% or more), but not more than about 75% (e.g., about 70%, about 65%, about 60%, about 55%, about 50% or less). In some embodiments, the IRES has a G-C content of at least 25%.

G-C content of a given nucleic acid sequence may be measured using any method known in the art, such as, for example chemical mapping methods (see, e.g., Cheng et al., PNAS, 114 (37): 9876-9881 (2017); and Tian, S. and Das, R., Quarterly Reviews of Biophysics, 49: e7 doi:10.1017/S0033583516000020 (2016)).

Exemplary sequences encoding IRESs for use in the circRNA molecules of the present disclosure are set forth in SEQ ID NOs: 138-17338. Thus, the disclosure further provides a recombinant circular RNA molecule comprising a protein-coding nucleic acid sequence and an IRES operably linked to the protein-coding nucleic acid sequence in a non-native configuration; wherein the IRES is encoded by any one of the nucleic acid sequences of SEQ ID NOs: 138-17338.

In some embodiments, the IRES is encoded by any one of the nucleic acid sequences set forth in SEQ ID NOs: 138-365. In some embodiments, the IRES is encoded by a nucleic acid sequence that has at least 80%, at least 85%, at least 90%, at least 91%, at least 92%, at least 93%, at least 94%, at least 95%, at least 96%, at least 97%, at least 98, or at least 99% identity to one or the nucleic acid sequences of SEQ ID NOs: 138-365. In some embodiments, the IRES is encoded by a nucleic acid sequence that has at least 1, at least 2, at least 3, at least 4, at least 5, at least 6, at least 7, at least 8, at least 9, at least 10 or more nucleotide substitutions relative to any one of the sequences in SEQ ID NOs: 138-365.

In some embodiments, the IRES is encoded by any one of the nucleic acid sequences set forth in SEQ ID NOs: 366-17338. In some embodiments, the IRES is encoded by a nucleic acid sequence that has at least 80%, at least 85%, at least 90%, at least 91%, at least 92%, at least 93%, at least 94%, at least 95%, at least 96%, at least 97%, at least 98, or at least 99% identity or homology to one or the nucleic acid sequences of SEQ ID NOs: 366-17338. In some embodiments, the IRES is encoded by a nucleic acid sequence that has at least 1, at least 2, at least 3, at least 4, at least 5, at least 6, at least 7, at least 8, at least 9, at least 10 or more nucleotide substitutions relative to any one of the sequences of SEQ ID NOs: 366-17338.

In some embodiments, the TRES is encoded by the nucleic acid sequences denoted Index 876 (SEQ ID NO: 668), 6063 (SEQ ID NO: 2407), 7005 (SEQ ID NO: 2739), 8228 (SEQ ID NO: 3179), or 8778 (SEQ TD NO: 3381). In some embodiments, the TRES is encoded by the nucleic acid sequence of SEQ TD NO: 33093.

In some embodiments, the IRES is encoded by any one of the nucleic acid sequences set forth in Table 5. In some embodiments, the TRES is encoded by a nucleic acid sequence that has at least 800%, at least 8500, at least 900%, at least 910%, at least 920%, at least 9300, at least 9400 at least 9500 at least 960%, at least 9700 at least 98, or at least 9900 identity or homology to one or the nucleic acid sequences of Table 5. In some embodiments, the IRES is encoded by a nucleic acid sequence that has at least 1, at least 2, at least 3, at least 4, at least 5, at least 6, at least 7, at least 8, at least 9, at least 10 or more nucleotide substitutions relative to any one of the sequences in Table 5.

TABLE 5
Illustrative Sequences Encoding IRES sequences

| Oligo- | | |
|---|---|---|
| nucleotide | | SEQ ID |
| Identifier | Sequence | NO: |
| 4173 | CATCCTCCTGCTGAAGTGACAAGCCACGCTGCTTCTGGA | 33091 |
| | GCCAAAGCTGACCAAGAAGAACAAATCCACCCCAGATC | |
| | TAGACTCAGGTCACCTCCTGAAGCCCTCGTTCAGGGTCG | |
| | ATATCCCCACATCAAGGACGGTGAGGATCTTAAAGACCA | |
| | CTCAACAGAAAGTAAAAAA | |
| 2125 | GCAGAAGGGAAACGTGAAGAAGGTGAAGATGGCGGTGG | 33092 |
| | CCAGGGCCGGGGTCTTGGGAGTCCAGTGGCTGCAAAGG | |
| | GCATCCCGGAACGTGATGCCGCTGGGCGCACGGACAGC | |
| | CTCCCACATGACCAAGGACATGTTCCCGGGGCCCTATCC | |
| | TAGGACCCCAGAAGAACGGGC | |
| 6742 | AGACTCGGGAACTGCCTGAATGTGGTTTGGGACACGAG | 33093 |
| | ACCTCATCATATTGATGAGCGAACAAACAAGAACATTTC | |
| | CTCCCTCCCCTCCTTTGAATTGAAATGGCACATTAAGACT | |
| | TGTCACGGCTTCTCACTGGGACTGGAGACCTCGTTCCTTC | |
| | ACCCCGCGTGTCGCCAG | |
| 8839 | AGGTGGGCGGTGGCGGTAGGTGGGTGGCCAGCGACTGG | 33094 |
| | CAACGCGGTGGCTTAGACGCGCGAGATCCGGCCAGGGT | |
| | CAGAGTACTGTAAGATTGATGTTAAAGGCATGGTGTTCA | |
| | CCCCACTTCATCAGCGTACATAAGTTATCTCTTCTTTTGG | |
| | ACCCTTATTTTATGCCATA | |
| 8279 | GGAGGCAGGTTGATGTGTTTGTGCTTCCTTCTACAGCCA | 33095 |
| | ATATGAAAAGGCCTAGTAAGTGGGGTCGGGAGGCGGGC | |
| | GTGGAGGGACCCACGTCTGGAAGTTGCTGCAGCCACCAC | |
| | GACGCTCTTCTACGGCTACGGCTTTGTCTCTGCTGAGTTA | |
| | AAGAAAGCAAGTAAACGC | |
| 1637 | ATTTATAAGCAATAGTAGCTTATTTGGCTCACAGTTCTG | 33096 |
| | GAGGCTGAGAAGATCGTGAGGCTGCATCTGGCAAGGGC | |
| | CTTCTTGCTGCTTCATAACATGGCAGAAGACATCATGCG | |
| | GGTGTGTGTCTGGGGAAGAGACTTACAGAAGTGGAGTT | |
| | GCTGAGTCAAAGATCTAACC | |
| 27147 | TCTTTTGGTGCAGAAGGTGACGGGAAACAGGCCGCAGA | 33097 |
| | CCTGAACTTCCAACCGTATGTAGGCGAGAAGCCGGTGCC | |
| | GATACTCCCACTATCCCACAATGTCCCACTGGGCCCCAG | |
| | AGTGGAAGAGGGCGGAGGCTAATCCAAGAGACCTTGGG | |
| | GCCAGCTGGGATGTCAGGGG | |
| 7408 | ATCCACCATGAAGCAGAATCTAAAGAAAAAGGTCCAAG | 33098 |
| | CATGAAGGAACCAAATCCAATTTCTCCACCAATTTTCCA | |
| | TGGTCTGGACACCCTAACCGTGATGGGCATTGCGTTTGC | |
| | AGCCTTTGTGATCGGAGCACTCCTGACGGGGGCCTTGTG | |
| | GTACATCTATTCTCACACA | |
| 29135 | CAGATTAGAGGACGGGCGCTTTGGAGCCGGCCCCAGGC | 33099 |
| | AGCGTGTGTCGGTCGCCTAGTCTGGAGAACTAGTCCTCG | |
| | ACTCACGTGCAAGGATGATGCTGAAAGGAATAACAAGG | |
| | CTTATCTCTAGGATCCATAAGGCCAAGCATGGGGATCAG | |
| | CACGAGGGTCAGCACTACAA | |
| 7806 | CTCTCGGGGGCCAGGGGTCATCGGTTTGACCCCTGACCT | 33100 |
| | ATAAGCCAAGATACCCCATAAACACACTCAGAAAGCAG | |
| | AGAAAAAGGACAAGAGTCTGTGTTTGAGAGGGGGTCTG | |
| | CCATTCCTGCTTGGGGACTGGTGGGGAAGAGGGCCAGG | |
| | ACATCTTCTGAGCCAGGACGT | |
| 661 | AGTCTGGCTGCGGCGGCAGAGCTGAAGTGAGCGGAGCC | 33101 |
| | ACCAGGAGGCCATGTCGGGTGAGGACGCTGAGGTCCGG | |
| | GCAGTCTCTGAAGATGTCTCCAATGGAAGCAGTGGCTCG | |
| | CCCAGCCCTGGGGACACACTGCCCTGGAACCTTGGGAAA | |
| | ACGCAGCGGAGCCGGCGCAG | |
| 1258 | ATCTGCCTCAGCCTCTTTGTCACTGCCTGGACCATTGTCC | 33102 |
| | CTGCTGTTTCTCAGACAGCATAGAGGCTGAGGCTGGGGC | |
| | CAGGACCCAGACAGAGACACACGGTCACTGCAGCTGAA | |
| | GCCGCTGCCCCTGCTACAGGCACCACCAGGACCAGCTGA | |
| | TCATTCCAGCCCACAGCA | |
| 6757 | CTCAGGCTGTGGCCCCACCTGCCCCCAAGCCCACCCAGG | 33103 |
| | CTGGGGAAGGAACGCTGTCAGAGGCCCTGCTGCAGCTG | |
| | CAGTTTGATGATGAAGACCTGGGGGCCTTGCTTGGCAAC | |
| | AGCACAGACCCAGCTGTGTTCACAGACCTGGCATCCGTC | |
| | GACAACTCCGAGTTTCAGC | |
| 5825 | CTCAAGGTTTTGAAGAGACAGGGGACACGCTCTATGCCC | 33104 |
| | CCTATTCCACACATTTTCAGCTGCAGAACCAGCCTCCCC | |
| | AAAAGGAATTCTCCATCCCACGGGCCCACGCTTGGCCGA | |
| | GCCCTTACAAAGACTATGAAGTAAAGAAAGAGGGCAGC | |
| | TAACTCTGTCCTGAAGAGT | |
| 27115 | GCTTCCGTAGAGGCCAGCTGGTTGCTACGCAGCAACAGG | 33105 |
| | AAACATTCCGCGCTACCGAGTACTTTCTACTCCCGACCA | |
| | GGCATTGCTCTCTCTGGAGACCCTCGGCGGTGGTTGCTG | |
| | TATTTTGACTTGGAGTAAGGATGACTTTTCGGGCCACAG | |
| | ATAGTGAATTTGACCTGA | |
| 3130 | CTGGAGGGCCGCACTCCCGTTCCAGCCAGGCTGAGCCTT | 33106 |
| | CTGTCCCCTGCCTCTGGGGCCTGGGAACCCCCCTTCTTCT | |
| | TTCTCCTGAATGGCACCCCCGCCCTAGAATCCAGACACC | |
| | GAGTTTCCCACTGTGGCTGGTTCAAGGAGCTCCCTGGTG | |
| | ACAGTCTGTGGCTGAGC | |
| 5065 | TAGAGTGGGGGGTGGAAGATGATTCTCGCCTGTTGCTGG | 33107 |
| | GGATTTATGAACATGGCTATGGAAACTGGGAGTTAATTA | |
| | AAACAGACCCAGAGCTTAAATTAACTGACAAAATTCTGC | |
| | CGGTGGAGACAGATAAAAAGCCTCAGGGGAAGCAGCTA | |
| | CAGACCCGAGCGGATTACT | |
| 8066 | AGCGGCTGCAGCATCCAGCCAGCTTGGATGTCTGGCCTG | 33108 |
| | TGAGCCTGGGGAAACTATTATTAATAATATTTACTGTTG | |
| | ATAATATTGGGGAAAACAGCCCTTAACTCTGAGGTTTCT | |
| | GCTGTGCTCCTTTCCAAAACAGACTTCCAGGACTCTGAA | |
| | GAAACAGTTACAAGCAGG | |
| 1952 | AAGGGAGGAGGCGCCGAGCTGACCGGGCGACGCCGCGG | 33109 |
| | GAGGTTCTGGAAACGCCGGGAGCTGCGAGTGTCCAGAC | |
| | ACTTCCCTCTGTGACCATGAAACTCTGGGTGTCTGCATTG | |
| | CTGATGGCCTGGTTTGGTGTCCTGAGCTGTGTGCAGGCC | |
| | GAATTCTTCACCTCTATTG | |
| 5020 | GCGAACTGAAGGTGGCCCAAGGGAAGGCTCTGCAAGAG | 33110 |
| | AATTTGGCCCTCCTGACCCAGACCCTAGCTGAAAGAGAA | |
| | GAGGAGGTGGAGACTCTGCGGGGACAAATCCAGGAACT | |
| | GGAGAAGCAACGGGAAATGCAGAAGGCTGCTTTGGAAT | |
| | TGCTGTCTCTGGACCTGAAGA | |
| 8766 | AGGAAGCCGAGGAAGGCTGTGTTTCTGACTCACACGGG | 33111 |
| | GGAGTCGGGGGAGTCGTAAACAACCCTGAAGAGAACAG | |
| | CCAGGCCTGGCGCTCTGGGCTTGGTCCTGGGACACCCGT | |
| | TTGACACTGTAAAGGTGAGGCTGCAGACCCAGACCACCT | |
| | ACCGGGGCATCGTTGATTGC | |
| 27433 | CTGCCTCCCCCCCGCTCCCGCTCCCCTGAGCCCAGCCAG | 33112 |
| | ACCCCGCGCCGCCCGCGCCCCGCTCGACTCCGGAGGCTC | |
| | CCGCAGCCCCGGCGTCCGCCCCGCTGCCCCCTCCCCCGG | |
| | GGGCCATGGGGGCGCCCCCGGGCTACCGGCCCTCAGCTT | |
| | GGGTGCATCTCCTCCACC | |
| 27818 | CTCAGTGAAGCAACGAGGATGCCGGGGAGAGGGAAGGG | 33113 |
| | GCTGGGCTCTGGGCGGTGCCAAGTCTGTGAGGGGGCGC | |
| | GGTCACCGCCCAGGGTTCCCACGAACGCCAAGGCGGCC | |
| | ACGTCCTGCTCCCCCTGGTGAAGAAGCTGCCCTGGGCTT | |
| | GTCGTCCTAGGGTCTCCAGAC | |
| 8861 | CTGATGTGAGCTAGTTTGTCTGGTTGAGTTGGATGTTTAA | 33114 |
| | ATAGAAGGCAGAACAACAACAGGTACTCCACATCAGCA | |
| | TTCTCAAGACTGGAGAAGTTAGGCCTCAGACATCCCAAG | |
| | CCTTCTCCTTTCATTGGAAACTTGACATTTTTCCGCCAGG | |
| | GTTTTTGGGAAAGCCAA | |
| 8040 | GCCACATTCCACACTAACGTGTTGGTGAATTCTTCTGGG | 33115 |
| | CATTGCCAGTACCTGCCTCCAGGCATATTCAAGAGTTCC | |
| | TGCTACATCGATGTACGCTGGTTTCCCTTTGATGTGCAGC | |
| | ACTGCAAACTGAAGTTTGGGTCCTGGTCTTACGGAGGCT | |
| | GGTCCTTGGATCTGCAG | |
| 27907 | TCCTGCCTCGGCAACCCCGGGCCCTGAGGGCAGGCCCCA | 33116 |
| | ACCGCGGAGGAGCAGGAGAGGGCGGAGGCCGGCGGGC | |
| | CATGCCCTGGTCGTCCCGCGGCGCCCTCCTTCGGGACCT | |
| | GGTCCTGGGCGTGCTGGGCACCGCCGCCTTCCTGCTCGA | |
| | CCTGGGCACCGACCTGTGGG | |
| 2392 | AAGTTTGAAACTGGTAACTTCGGGAGTTGAGCCACGAGC | 33117 |
| | TGTTGTGCATCCAGAGGTGGAATTGGGGCCCGGCATTCC | |
| | CTCCTCGTCCCGGGCTGGCCCTTGCCCCCACCCTGCAACT | |
| | CCTGGTTGAGATGGGCTCAGCCAAGAGCGTCCCAGTCAC | |
| | ACCAGCGCGGCCTCCGC | |
| 7802 | AGCCTTCGCTGGATCTCCTTCCCGAACTCACAAATCCTG | 33118 |
| | ACGAGCTCCTGTCTTATCTGGACCCCCCCGACCTGCCGA | |
| | GCAATAGTAACGATGACCTCCTGTCTCTATTTGAGAACA | |
| | ACTGAGGGCCACCCGGTCGGGGCCATCCCTCCACACTCT | |
| | GCATCCTACCCCACCTAC | |
| 7655 | TTTTTAGAGGTTGGTTGTTGTGTTTTAGGATTCTGTCCAT | 33119 |
| | TTTCTTTTAAAGTTATAAACACGTACTTGTGCGAATTATT | |
| | TTTTTAAAGTGATTTGCCATTTTTGAAAGCGTATTTAATG | |
| | ATAGAATACTATCGAGCCAACATGTACTGACATGGAAA | |
| | GATGTCAAAGATATGT | |
| 981 | AGCCAGCGGACGTCCAGGAACCGGGATGCCTCCAGCAG | 33120 |
| | TGAGGCGGTCAGCCTGCAGCATGGGATGGCTGTGGATCT | |
| | TTGGGGCAGCCCTGGGGCAGTGTCTGGGCTACAGTTCAC | |
| | AGCAGCAAAGGGTGCCATTTCTTCAGCCTCCCGGTCAAA | |
| | GTCAACTGCAAGCGAGTTA | |
| 5027 | GGGACCAGGAACTGGAGGCTCTGCAGCAAGAACAGCAG | 33121 |
| | CAGGCCCAGGGACAGGAGGAGAGGGTGAAGGAAAAGG | |
| | CAGACGCCCTCCAGGGAGCTCTGGAGCAAGCCCATATG | |
| | ACACTGAAGGAGCGTCATGGAGAGCTTCAGGACCACAA | |
| | GGAACAGGCACGAAGGCTGGAGG | |
| 7683 | CTACTCCGGAGCCTGAGGTGGGAGGATCGCTTGAGTCTG | 33122 |
| | GGAGGCAGAGGCTGCATTGAGCTATGATCATGGCACTGC | |
| | ATTCCAGCCTGGGTGACAGTGCAAGACCTTGTCTCAGAA | |
| | TAAATAAAGTATGTGATGAAGATGTGCATACATTATATG | |
| | CAAATACTGTTTTTTTTT | |
| 8650 | CACCAATTCGGCTGGCGTCTCCGAGACCGCGGACTCCCG | 33123 |
| | TAGGGTCCCCGTGGCCCCGAGTTGTAGTCGGGACACCCC | |
| | GGCCGCGGGTGATCGTCGGGTCTCCACGCGCCCGGGTCG | |
| | CTGACGCGGATCCGGCCTCGGCGCCTTCTCAGGGCGCCC | |
| | TGCAAGGCCGCAGGCAGG | |
| 6898 | CCTCCTCTCTGGCTAATCAACAGCTGCCCCCAGCCTGTG | 33124 |
| | GTGCCAGGCAACTCAGCAAGCTGAAAAGGTTCCTTACTA | |
| | CCCTGCAGCAGTTTGGCAATGACATTTCACCCGAGATAG | |
| | GAGAAAGAGTTCGCACCCTCGTTCTGGGACTAGTGAACT | |
| | CCACTTTGACAATTGAAG | |
| 2042 | TTTAGTGTCACAGGAGACACGAGGGCAAAGTGCTGAGG | 33125 |
| | AAACTCTAGAGCAACATCGGAATGCATGGTGAAGCATC | |
| | AGATGGTGAAGGAGAAGGAGACACAGAAGTGATGCAGC | |
| | AGGAGACAGTTCCAGTTCCTGTACCTTCAGAGAAAACCA | |
| | AACAGCCTAAAGAATGTTTTT | |
| 1686 | GCCACTTCTCTTCCCTTCATTCTTCGCCAGGCTCTCTGCT | 33126 |
| | GACTCAAGTTCTTCAGTTCACGATCTTCTAGTTGCAGCG | |
| | ATGAGTGCACGAGTGAGATCAAGATCCAGAGGAAGAGG | |
| | AGATGGTCAGGAGGCTCCCGATGTGGTTGCATTCGTGGC | |
| | TCCCGGTGAATCTCAGCA | |
| 5902 | CTTCAACTTGGCAACAGTTGCCTGGGGTAGCTCTACACA | 33127 |
| | ACTCTGTCCAGCCCACAGCAATGATTCCAGAGGCCATGG | |
| | GGAGTGGACAGCAGCTAGCTGACTGGAGGAATGCCCAC | |
| | TCTCATGGCAACCAGTACAGCACTATCATGCAGCAGCCA | |
| | TCCTTGCTGACTAACCATG | |
| 3399 | CACCCTGACCCAAGCCGAGACAGGTTCCAAACCTCAACC | 33128 |
| | TGCAGCCGGAAGGGGGAAGTGAAACTCGGCTGGGGGTG | |
| | GGGGCTCAGAAGCCGCCCCAGAAAGCACTGAAAGCCAC | |
| | AGCACGTACACCCACTCCAGGGATCTGCCAGCACCCTGT | |
| | GGGGCCCAGACTACAGGCTG | |
| 6560 | GGGACGCCATTGTGATTGGGGGGGGATCCAGGGCTGCT | 33129 |
| | TCACTGCATACCACCTGGCCAAACACAGGAAGAGGATC | |
| | CTCCTGCTGGAGCAGTTCTTTCTACCACACTCCCGAGGA | |
| | AGCTCCCATGGACAAAGCCGGATAATCCGAAAGGCGTA | |
| | CCTGGAAGACTTTTACACCC | |
| 2890 | CACCGTAGTGCTTAGAGGCCGAAAAAGTACAGCCCCTTC | 33130 |
| | CGGGCTCCGCGGTACGGGAAGACAGCTTTGGGATGTCG | |
| | GAAGATCCTAGAGGTCCCAAGGTACCACGAAAGGCAGT | |
| | GGTAGAAAAGCCAGCTCGGGCAGCAGAGCGAGAGGCCC | |
| | GGGCCCTGCTGGAGAAGAACC | |
| 4343 | ATTGGCTGAGCCCGGCTGTCAGTCCTTTCGCGCCTCGGC | 33131 |
| | GGCGCGGCATAGCCCGGCTCGGCCTGTAAAGCAGTCTCA | |
| | AGCCTGCCGCAGGGAGAAGATGGCGGTCGCCGTGAGAA | |
| | CTTTGCAGGAACAGCTGGAAAAGGCCAAAGAGAGTCTT | |
| | AAGAACGTGGATGAGAACAT | |
| 6138 | ACAAGGAGACTGGGAGGTGTCTCAAGTGCCTGTACCAC | 33132 |
| | ACGGAAGGGGAACACTGTCAGTTCTGCCGGTTTGGATAC | |
| | TATGGTGATGCCCTCCAGCAGGACTGTCGAAAGTGTGTC | |
| | TGTAATTACCTGGGCACCGTGCAAGAGCACTGTAACGGC | |
| | TCTGACTGCCAGTGCGACA | |
| 2241 | GAGGAAGGAGAGAGTGGAGGAGGAGGGCTTTGGGTTAG | 33133 |
| | GGAGAGTGCTTTCGTTTGTTTTAAATGGGAGAAACTGGA | |
| | GCATGTTGCCAAGGGCAGAGAGCCAGCAGAGAGGGGTG | |
| | AATGGAAGAAGGAGCGAGAAGGGGGTTACTGACGAAGC | |
| | CTTATCCTGGAGGAGAGAAGG | |
| 5715 | CAAGTGGTCCTGTCGAAGTATTTATCACTGAGACTCCGA | 33134 |
| | GTCAGCCCAACTCCCACCCCATCCAGTGGAATGCACCAC | |
| | AGCCATCTCACATTTCCAAGTACATTCTCAGGTGGAGAC | |
| | CTAAAAATTCTGTAGGCCGTTGGAAGGAAGCTACCATAC | |
| | CAGGCCACTTAAACTCCT | |
| 3456 | CTAGACCAAAGGGTGCGGCTGCTGCAGAGGTGGCTGAT | 33135 |
| | GCAGGTTTCCATCCTTGGGGAATGACCACGCAACTGGGC | |
| | CCAGCCCTGGTGCTGGGGGTGGCCCTGTGCCTGGGTTGT | |
| | GGCCAGCCCCTACCACAGGTCCCTGAACGCCCCTTCTCT | |
| | GTGCTGTGGAATGTACCCT | |
| 5260 | ATCTCCAGCCATTGCTCCTCCAACAGAGACCATGGCTCC | 33136 |
| | TCCAGTCAGGGATCCTGTTCCTGGGAAACCCACGGTCAC | |
| | CATCCGGACTCGAGGCGCCATTATTCAAACCCCAACCCT | |
| | AGGCCCCATCCAGCCTACTCGGGTGTCAGAAGCTGGCAC | |
| | CACAGTTCCTGGCCAGAT | |
| 1288 | GGAGGCAGGCGGTGCCGCGGCGCCGGGACCCGACTCAT | 33137 |
| | CCGGTGCTTGCGTGTGGTGGTGAGCGCAGCGCCGAGGAT | |
| | GAGGAGGTGCAACAGCGGCTCCGGGCCGCCGCCGTCGC | |
| | TGCTGCTGCTGCTGCTGTGGCTGCTCGCGGTTCCCGGCG | |
| | CTAACGCGGCCCCGCGGTCG | |
| 8756 | CACCCCCACCGACCTCTCGGAGACAGGAATCCGCTCTGC | 33138 |
| | CCCTGCATCCTCCTCTGCTCACCCTTCTCTCAGTAGTGTT | |
| | TGTCAGACACCAGTCCGGACCTCAGCTCTCGATCTAGTT | |
| | CCCGAGTAAGATTTAGCTCCCGGGAAAGCGTGCCTGAAA | |
| | CAAGCCGGAGTGAGCCT | |
| 8952 | CCTGTACCATAACCAGGAGGCTGATGGTTGCTCAGGCCT | 33139 |
| | CCCTCAAACTAGCCAATGAGAAGTGCACGGGTCCTGGTC | |
| | GCCTAGGAAACCGCGTGACAACAAGATGGCGGCGCTGC | |
| | GGGACGGCTAGCGGCCCTGCGTGGGAGTTCCTGACAGCT | |
| | GGATTCTAGAAGTAGAACT | |
| 2759 | TTCCCCGGAAGTGTTGGTTAAAGCCCCTCCAATCAGCGG | 33140 |
| | CTCGGTGCGGCAAGTTTGAATTTCGTGGAGGCTCGGGTT | |
| | GTGAGGGTTCCTGCTTCGGAGTCGGCGGTGGTCGTCCAG | |
| | ACCGAGTGTTCTTTACTTTTTGTTTGGTTGAGGTTTCACG | |
| | CTAGAAGGTGGCTCAGG | |
| 1241 | CTGTCCATACACTCTCTCATCATCCTGTTCCTTGGATTGG | 33141 |
| | ACTTCACTAAGCAATTTATCACTCACCTTCAGACTTACAT | |
| | GTGGGAGTTTTCACAACAGTAGTTTTGGAATCATTAGAA | |
| | CTTGGATTGATTTCATCATTTAACAGAAACAAACAGCCC | |
| | AAATTACTTTATCACC | |
| 28013 | GAGCTCCAGCCTCCAGGCACCCGGGATCCAGCGCCGCCG | 33142 |
| | CTCATAACACCCGCGACCCCGCAGCTAAGCGCAGCTCCC | |
| | GACGCAATGGACCCGGCGCTGGCAGCCCAGATGAGCGA | |
| | GGCTGTGGCCGAGAAGATGCTCCAGTACCGGCGGGACA | |
| | CAGCAGGCTGGAAGATTTGC | |
| 4681 | TGTTGACTCATGCAAATGAGGTATCTGAACTGCAGCTTC | 33143 |
| | AGTATTAGCAGAGCCACAGGCCGCCTCTGTGGCATCACC | |
| | AGGGTTTCTCTGAAGAAGAGGGTCTGCATTTTCCTAAAC | |
| | CCAGTGCTGCTCTCCCATCTCCCATCTTCCTCTCGCAGCT | |
| | TGATGAGCCCCGGTGTG | |
| 5602 | TGCCCCAGCCCTCAGGGCCGCCAGCAAGGAAGAAATTT | 33144 |
| | GTGATACCCCTCGACGAGGATGAGGTCCCTCCTGGAGTG | |
| | GCCAAGCCCTTATTCCGATCTACACAGAGCCTTCCCACT | |
| | GTGGACACCTCGGCCCAGGCGGCCCCTCAGACCTACGCC | |
| | GAATATGCCATCTCACAGC | |
| 1945 | GAGAGGCCGGCGTCTCTCCCCCAGTTTGCCGTTCACCCG | 33145 |
| | GAGCGCTCGGGACTTGCCGATAGTGGTGACGGCGGCAA | |
| | CATGTCTGTGGCTTTCGCGGCCCCGAGGCAGCGAGGCAA | |
| | GGGGGAGATCACTCCCGCTGCGATTCAGAAGATGTTGGA | |
| | TGACAATAACCATCTTATT | |
| 5229 | AACCCCAACCCTAGGCCCCATCCAGCCTACTCGGGTGTC | 33146 |
| | AGAAGCTGGCACCACAGTTCCTGGCCAGATTCGCCCAAC | |
| | GATGACCATTCCTGGCTATGTGGAGCCTACTGCAGTTGC | |
| | TACCCCTCCCACAACCACCACCAAGAAGCCACGAGTATC | |
| | CACACCAAAACCAGCAAC | |
| 7542 | CTGCAGCATCTTCTACGTCGGGACCACAGCCTCCGCCTC | 33147 |
| | CACAAAGCCTGAACCTCCTTTCGCAGGCTCAGCTGCAGG | |
| | CACAGCCTCTTGCGCCAGGCGGAACTCAAATGAAAAAG | |
| | AAAAGTGGCTTCCAGATAACTAGCGTTACTCCTGCTCAG | |
| | ATCTCCGCTAGTATCAGCT | |
| 6866 | CCAATACCTGGGATCCATTGCCTCTCCTTCTGTGCACCCA | 33148 |
| | GCAACGCCCATTTCACCTGGACGTGCCAGCGGCATGACA | |
| | ACCCTCTCTGCAGAACTTTCCAGTCGACTCTCAACGGCA | |
| | CCCGACCTGACAGCGTTCAGCGACCCGCGCCAGTTCCCC | |
| | GCGCTGCCCTCCATCTC | |
| 1849 | TCTCTTGCTTCAACAGTGTTTGGACGGAACAGATCCGGG | 33149 |
| | GACTCTCTTCCAGCCTCCGACCGCCCTCCGATTTCCTCTC | |
| | CGCTTGCAACCTCCGGGACCATCTTCTCGGCCATCTCCTG | |
| | CTTCTGGGACCTGCCAGCACCGTTTTTGTGGTTAGCTCCT | |
| | TCTTGCCAACCAACC | |
| 2558 | AGTTACCTCTCCCCTTTCACGTAGTTTTCATTTGTGGTGA | 33150 |
| | GATTCTCTCCCAGGCCACAAGACATTTCCTGCTCGGAAC | |
| | CTTGTTTACTAATTTCCACTGCTTTTAAGGCCCTGCACTG | |
| | AAAATGCAAGCTCAGGCGCCGGTGGTCGTTGTGACCCAA | |
| | CCTGGAGTCGGTCCCG | |
| 2109 | ATCAGCCCAAAGGTAGGCTCAGGCTCCGACGGTGGCCG | 33151 |
| | GCGGGGGTCACGAGGCTTCGTAGTGGAGGAACGGGTTT | |
| | GGCGTGTGGGACGCAGCTGCCTCTGTACTGGGGAGTCAC | |
| | GGAGTGGCCGGGCTCCAGGGACATGGCGGCGGCCTCTG | |
| | CGGTGTCGGTGCTGCTGGTGG | |
| 297 | ATCCGGAGGCAGCCTGCAGGAAGCCGTAGCGCCGGTAC | 33152 |
| | GTGCCCCTCTCCTGTCTGGAGGCGGGTGTAGAAGTCCGA | |
| | CCGCGGAAGCCAGACTGCTGTCCAGTCGGCGAGCGCGT | |
| | ACCATTCAGCATCGGCTCCGCCCGAGTCCCACCTTCCTC | |
| | AGGCTCTGATTGGCTGACAC | |
| 2621 | CAACAGAAGCCAAGAAGGAAGCCGTCTATCTTGTGGCG | 33153 |
| | ATCATGTATAAGCTGGCCTCCTGCTGTTTGCTTTTCATAG | |
| | GATTCTTAAATCCTCTCTTATCTCTTCCTCTCCTTGACTCC | |
| | AGGGAAATATCCTTTCAACTCTCAGCACCTCATGAAGAC | |
| | GCGCGCTTAACTCCGG | |
| 6574 | TGGCTGACACATTCCTGGAGCACATGTGCCGCCTGGACA | 33154 |
| | TTGATTCACCACCCATCACAGCCCGGAACACTGGCATCA | |
| | TCTGTACCATTGGCCCAGCTTCCCGATCAGTGGAGCTGA | |
| | AGAAGGGAGCCACTCTCAAAATCACGCTGGATAACGCC | |
| | TACATGGAAAAGTGTGACG | |
| 1033 | GACATTTACATCCTGCCCAGCGACAACTCTGGACAAGTC | 33155 |
| | AGTCCCCCAGAGTCTCCAACTGTGACCACTTCCTGGCAG | |
| | TCTGAGAGCTTACCTGTGTCACTGTCAGCTAGCCAGAGT | |
| | TGGCACACAGAAAGCCTGCCAGTGTCACTAGGCCCTGAG | |
| | TCCTGGCAGCAGATTGCA | |
| 8789 | CCTCTGACTTGACCCAATGAAAGAAGCATATGGCACTTG | 33156 |
| | TGAAGATAAATGTTACTCCTCCCTTTTTAATTGGAACTTC | |
| | TGCTTAGGACCTGTGTATGACGTTTCACCTGTGATCTGTT | |
| | CTTTCGGTAGCCACTGACTTTGAGTTACAGGAAGGTCTC | |
| | CGAAGATTTGTGTCAA | |
| 6392 | CTTCCCTGAGGAGGACGACCCCACCAACTGGCTGCGTTG | 33157 |
| | CTACTACTACGAAGACACCATCAGCACCATCAAGTCTGT | |
| | GGCCTGGGAGGGAGGGGCCTGTCCAGCCTTCCTGCCATC | |
| | CCTACGACCACTGCCCCTCACATCACCTTCTCATGGGTCC | |
| | CTCTCCCACTCCAAAGC | |
| 6885 | ATTTATGATTTCAACATGGATACATATTTCAGTTCTTTCT | 33158 |
| | TTTTCTCACTATCTGAAAATACATTTCCCTCCCTCTCTTC | |
| | CCCCCAATATCTCCCTTTTTTTCTCTCTTCCTCTATCTTCC | |
| | AAACCCCACTTTCTCCCTCCTCCTTTTCCTGTGTTCTCTTA | |
| | AGCAGATAGCAC | |
| 561 | CCCTTCCGGCTGGCCCCGCTCAGTCACCCGCAGCAGGCG | 33159 |
| | TGCAGTTTCCCGGCTCTCCGCGCGGCCGGGGAAGGTCAG | |
| | CGCCGTAATGGCGTTCTTGGCGTCGGGACCCTACCTGAC | |
| | CCATCAGCAAAAGGTGTTGCGGCTTTATAAGCGGGCGCT | |
| | ACGCCACCTCGAGTCGTG | |
| 7556 | ATACAAATTTGCCTTTGGCACAACAGATACCACTAAGTT | 33160 |
| | CTACCCAGTTCTCCGCACAATCATTAGCTCAGGCAATTG | |
| | GAAGCCAAATTGAAGATGCCAGGCGTGCAGCGGAGCCC | |
| | TCCTTAGTTGGCTTACCTCAGACTATCAGTGGTGACAGT | |
| | GGGGGAATGTCAGCAGTTT | |
| 8800 | TCCGTCCTCCAGGATCTGGGGAGAAAGAGCCCCATCCCT | 33161 |
| | TCTCTCTCTGCCACCATTTCGGACACCCCGCAGGGACTC | |
| | GTTTTGGGATTCGCACTGACTTCAAGGAAGGACGCGAAC | |
| | CCTTCTCTGACCCCAGCTCGGGCGGCCACCTGTCTTTGCC | |
| | GCGGTGACCCTTCTCTC | |
| 5781 | GTTTACTTCCCCTGCCAGCATTGGGGTGCTCTCTAAGCA | 33162 |
| | ACAGTAGGCGGAGAGTGGTCTGGCGTATTAAAAACAAA | |
| | GGATCGTCAAGTGGGCCTTCCCAGGCATTGCTTTGACTT | |
| | AGTACATGTAGAGGATGTGGCAGTTCTCTCCGTCCCTGC | |
| | CACTGCTGGTTTCTTTGTT | |
| 2930 | TATTTTAAGCCCAGTCTTCCCTGGGCCACCTTTAGCAGAT | 33163 |
| | CCTCGTGCGCCCCCGCCCCCTGGCCGTGAAACTCAGCCT | |
| | CTATCCAGCAGCGACGACAAGTAAAGTGGCCCGCCGGTT | |
| | TCTGAGCCTTCTGCCCTGCGGGGACACGGTCTGCACCCT | |
| | GCCCGCGGCCACGGACC | |
| 3426 | CCAGCGGAAGTAATTCTTTCGACTGCCCCGGAACCCACC | 33164 |
| | GGAGCAGGCAGCTGGGGGTGGGGGGGCGGCCCTGGGAT | |
| | AGGGGCTGTGGCAGTACGCGGGGACCCGGCTGCGGTGG | |
| | CTGCGGGACTGACGAATGGAGAGGAGAAAACCTATGGT | |
| | GGCTGTGAAGGACCTGATGCC | |
| 5800 | ATGGGGAAGGTGAAGGTCGGAGTCAACGGATTTGGTCG | 33165 |
| | TATTGGGCGCCTGGTCACCAGGGCTGCTTTTAACTCTGG | |
| | TAAAGTGGATATTGTTGCCATCAATGACCCCTTCATTGA | |
| | CCTCAACTACATGGTTTACATGTTCCAATATGATTCCACC | |
| | CATGGCAAATTCCATGGC | |
| 27433 | CTGCCTCCCCCCCGCTCCCGCTCCCCTGAGCCCAGCCAG | 33166 |
| | ACCCCGCGCCGCCCGCGCCCCGCTCGACTCCGGAGGCTC | |
| | CCGCAGCCCCGGCGTCCGCCCCGCTGCCCCCTCCCCCGG | |
| | GGGCCATGGGGGCGCCCCCGGGCTACCGGCCCTCAGCTT | |
| | GGGTGCATCTCCTCCACC | |
| 424 | ATTTCTGGGAAGCAGTCGATATTATCTGTACGCCTAGAA | 33167 |
| | CAGTGCCCTCTGCAGCTGAATAACCCTTTTAACGAGTAT | |
| | TCCAAATTTGATGGCAAGGGTCATGTAGGTACAACAGCA | |
| | ACCAAGAAGATCGATGTCTACCTCCCTCTGCACTCGAGC | |
| | CAGGACAGACTGCTGCCA | |
| 28259 | CTCTGGTGATACAGAAGAAAAGACAGTCTCCATTTTCAA | 33168 |
| | ACAGTCCCTCCTGGGAGAACACAGACAGGCAGAGGATT | |
| | ACAACACAAGGCAGCAAGCACTGGGAGACGAAAGTTTT | |
| | GGCATCTGTTCCCTGGCTGTGCCAAGATGGGCGATTGGA | |
| | GCTTCCTGGGAAATTTCCTG | |
| 6972 | GTCTACTATGTCTGAACTGTGGTTTCTTGTTTATCCTTTTT | 33169 |
| | TCCTTAGTTGGACTGTAATGTATGGTCTGTCAACCTGTGA | |
| | ATCTTTAAAGTATGATTCAGGTATTGTTGTATTCTTTACT | |
| | GTGTAATAAAAAAGTTGAAAAAAATCTGGATCCTCTGTC | |
| | TCCCTCGTCCCCGG | |
| 417 | TTTCGTGACCTTGTCCAGTAGAAGGCTATTTAATTTTCAC | 33170 |
| | AACTGCTTGAATTTTGACATACAAGATGAAGCAAGATGC | |
| | CTCAAGAAATGCTGCCTACACTGTGGATTGTGAAGATTA | |
| | TGTGCATGTGGTAGAATTTAATCCCTTTGAGAATGGGGA | |
| | TTCAGGAAACCTAATTG | |
| 8182 | TGTCTGCACCAGCCGGCCTCCAGCCTGGCTGGACCCTGC | 33171 |
| | TGCCTGTGTGGCCCGGAGCCAGAGGCCCCCACACTCCCA | |
| | GCTGCTCTTCTACAGATGCCATCAACGAGCAGGACTCTG | |
| | GGTGGCTCCACTGTCTAAGGGCTCTGCGAGTGACCCGGC | |
| | GGGCGAGCTCCGTGCTGC | |
| 7572 | CTTACGTAGTAATTACATTTCCTTGAAAAAACTATAGTG | 33172 |
| | AATAGAAATCCCTAGCCATTTCATTTTTTATGTTTTTAAT | |
| | GAAGATCTTTAAAATACCATAGGTGGTAATCGTGGAAAA | |
| | TTTGAAAAATCTCATGTCAGTGTATTAAGATGGTGGAGA | |
| | AGTTTTTTTCTCCATTA | |
| 1415 | ATTCATGAAAATCCACTACTCCAGACAGACGGCTTTGGA | 33173 |
| | ATCCACCAGCTACATCCAGCTCCCTGAGGCAGAGTTGAG | |
| | AATGGAGAGAATGTTACCTCTCCTGGCTCTGGGGCTCTT | |
| | GGCGGCTGGGTTCTGCCCTGCTGTCCTCTGCCACCCTAA | |
| | CAGCCCACTTGACGAGGA | |
| 7327 | GAGCTTCCAGGGATATTTGAGGCACCATCCCTGCCATTG | 33174 |
| | CCGGGCACTCGCGGCGCTGCTAACGGCCTGGTCACATGC | |
| | TCTCCGGAGAGCTACGGGAGGGCGCTGGGTAACCTCTAT | |
| | CCGAGCCGCGGCCGCGAGGAGGAGGGAAAAGGCGAGC | |
| | AAAAAGGAAGAGTGGGAGGA | |
| 705 | GCCCCCTCCCCTGACTATCAAAGCAGCGGCCGGCTGTTG | 33175 |
| | GGGTCCACCACGCCTTCCACCTGCCCCACTGCTTCTTCGC | |
| | TTCTCTCTTGGAAAGTCCAGTCTCTCCTCGGCTTGCAATG | |
| | GACCCCAACTGCTCCTGCGCCGCTGGTGTCTCCTGCACC | |
| | TGCGCTGGTTCCTGCA | |
| 8561 | AGTCGCATAGTGGTTTTTCCGCTCGCGTCGCTGTGTGAA | 33176 |
| | AGTTGGCTCGCCGCTCTTTGCACGCCCTCCCTGGAGGCC | |
| | GACCCGAGACGCCAAGCTGGAGAGACCGTGCCTCCCCG | |
| | AGGCCGGCCGCCCCGCGAGCACAGCCTCCGCCCCCGTTG | |
| | CACTGCCGGGCTGGGCAAT | |
| 1679 | GGTGGCGGGAAGAGGAGGCGCGAGAATGGAGGTGGAG | 33177 |
| | GCCGTCTGTGGTGGCGCGGGCGAGGTGGAGGCCCAGGA | |
| | CTCTGACCCTGCCCCTGCCTTCAGCAAGGCCCCCGGCAG | |
| | CGCCGGCCACTACGAACTGCCGTGGGTTGAAAAATATAG | |
| | GCCAGTAAAGCTGAATGAAAT | |
| 5635 | TATTTCCCTCTACTGAGTAAACCCTATCTGTGATTCCCCC | 33178 |
| | AAACATCTGGCATGGCTCCCTTTTGTCCTTCCTGTGCCCT | |
| | GCAAATATTAGCAAAGAAGCTTCATGCCAGGTTAGGAA | |
| | GGCAGCATTCCATGACCAGAAACAGGGACAAAGAAATC | |
| | CCCCCTTCAGAACAGAGG | |
| 5583 | TGTTACTTAAGAATGCTTTCCAGGTGGAAAGTTCCTTAA | 33179 |
| | GTTTGAGGCTTCAAATTCCATACAGCACATTAAAATCCC | |
| | ATTCATGAGTTTGAAATACTGCTCTGTTGTCTTGGAAATA | |
| | CCAATCAGATTGTTGGCTGAAGTGATGTGGATAAAGAAG | |
| | GGATCTTAGAAAAACTA | |
| 9027 | ACGAAGTTCCGGAAATGGAATTACCGAGTCAGATGAGA | 33180 |
| | AACACAAGTCATCAATGAATTAACAAGCAACACATTGTC | |
| | ACATCCAGCTGTTGAAGAGTGGTTAACTCAGCTTGATCT | |
| | CCGAAGACTCTTCTGCACCCCACCCCGCCCCCAACTGCT | |
| | TTCCAAAGAGAAACAGAAA | |

The IRES may be of any length or size. For example, the IRES may be about 100 nucleotides to about 600 nucleotides in length (e.g., about 200, about 225, about 250, about 275, about 300, about 325, about 350, about 375, about 400, about 425, about 450, about 475, about 500, about 525, about 550, or about 575 nucleotides in length, or a range defined by any two of the foregoing values). In some embodiments, the IRES may be about 200 nucleotides to about 800 nucleotides in length (about 200, about 210, about 220, about 240, about 260, about 280, about 320, about 340, about 360, about 380, about 420, about 440, about 460, about 480, about 500, about 520, about 540, about 560, about 580, about 600, about 620, about 640, about 660, about 680, about 700, about 720, about 740, about 760, about 780, or about 800 nucleotides in length, or a range defined by any two of the foregoing values). In some embodiments, the IRES may be about 200 to about 400, about 400 to about 600, about 600 to about 700, or about 600 to about 800 nucleotides in length. In some embodiments, the IRES is about 210 nucleotides in length. In some embodiments, the IRES may be about 100 to about 3000 nucleotides in length.

In some embodiments, a circular RNA molecule comprises of an IRES sequence that consists of a sequence encoded by a DNA sequence from SEQ ID NOs: 138-17338. In some embodiments, a circular RNA molecule comprises an IRES sequence encoded by a DNA sequence from SEQ ID NOs: 138-17338, wherein the IRES sequence additionally comprises up to 1000 additional nucleotides. In some embodiments, the IRES sequence is encoded by a sequence from SEQ ID NOs: 138-17338 and additionally comprises up to 1000 additional nucleotides located at the 5′ end of that sequence. In some embodiments, the IRES sequence is encoded by a sequence from SEQ ID NOs: 138-17338 and additionally comprises up to 1000 additional nucleotides located at the 3′ end of that sequence. In some embodiments, the IRES sequence is encoded by a sequence from SEQ ID NOs: 138-17338 and additionally comprises up to 1000 additional nucleotides located at the 5′ end of that sequence and up to 1000 additional nucleotides located at the 5′ end of that sequence.

In some embodiments, a circular RNA molecule comprises an internal ribosome entry site (IRES) sequence region, wherein the IRES sequence region comprises a sequence encoded by a DNA sequence from SEQ ID NOs: 138-17338, and wherein the sequence encoded by a DNA sequence from SEQ ID NOs: 138-17338 has a minimum free energy (MFE) of less than -18.9 kJ/mol and a melting temperature of at least 35.0° C.

In some embodiments, a circular RNA molecule comprises an internal ribosome entry site (IRES) sequence region, wherein the IRES sequence region comprises a sequence encoded by a DNA sequence from SEQ ID NOs: 138-17338, and wherein the IRES sequence region has a minimum free energy (MFE) of less than −18.9 kJ/mol and a melting temperature of at least 35.0° C., over its entire length.

In some embodiments, a circular RNA molecule comprises an internal ribosome entry site (IRES) sequence region, wherein the IRES sequence region comprises a sequence encoded by a DNA sequence from SEQ ID NOs: 138-17338, and additionally comprises up to 1000 additional nucleotides located at the 5′ end of and up to 1000 additional nucleotides located at the 5′ end, and wherein the IRES sequence region has a minimum free energy (MFE) of less than −18.9 kJ/mol and a melting temperature of at least 35.0° C., over its entire length.

In some embodiments, the recombinant circular RNA molecule comprises a protein-coding nucleic acid sequence operably linked to the IRES, optionally in a non-native configuration. Any protein or polypeptide of interest (e.g., a peptide, polypeptide, protein fragment, protein complex, fusion protein, recombinant protein, phosphoprotein, glycoprotein, or lipoprotein) may be encoded by the protein-coding nucleic acid sequence. In some embodiments, the protein coding-nucleic acid sequence encodes a therapeutic protein. Examples of suitable therapeutic proteins include cytokines, toxins, tumor suppressor proteins, growth factors, hormones, receptors, mitogens, immunoglobulins, neuropeptides, neurotransmitters, and enzymes. Alternatively, the protein-coding nucleic acid sequence can encode an antigen of a pathogen (e.g., a bacterium, virus, fungus, protist, or parasite), and the circRNA can be used as, or as one component of, a vaccine. Therapeutic proteins, and examples thereof, are further described in, e.g., Dimitrov, D. S., Methods Mol Biol., 899: 1-26 (2012); and Lagasse et al., F1000Research, 6: 113 (2017).

Ideally, the IRES is “in-frame” with respect to the protein-coding nucleic acid sequence, that is, the IRES is positioned in the circRNA molecule in the correct reading frame for the encoded protein. Examples of IRES elements that were found to be in-frame with one or more coding sequences are set forth in SEQ ID NOs: 29114-33083. In some embodiments, however, the IRES may be “out of frame” with respect to the protein-coding nucleic acid sequence, such that the position of the IRES disrupts the ORF of the protein-coding nucleic acid sequence. In other embodiments, the IRES may overlap with one or more ORFs of the protein-coding nucleic acid sequence. In addition, while in some embodiments the protein-coding nucleic acid sequence comprises at least one stop codon, in other embodiments the protein-coding nucleic acid sequence may lack a stop codon. The instant inventors have found that a circRNA molecule comprising a protein-coding nucleic acid sequence having an in frame non-native IRES and lacking a stop codon can initiate a recursive (i.e., infinite loop) translation mechanism. Such recursive translation may produce a concatenated protein multimer (e.g., >200 kDa). This particular circRNA design allows for the production of repeating ORF units up to 10 times the size of the single ORF. Without being bound to any particular theory, use of the circRNAs described herein for recursive gene encoding may represent a novel “data compression” algorithm for genes, addressing the gene size limitation associated with many current gene therapy applications.

In some embodiments, the IRES comprises (i) at least one RNA secondary structure element and (ii) a sequence that is complementary to an 18S rRNA. In some embodiments, the IRES comprises (i) at least one RNA secondary structure element and (ii) a sequence that is complementary to an 18S rRNA, wherein the RNA secondary structure of the IRES is formed from the nucleotides at about position 40 to about position 60 of the IRES, wherein the first nucleic acid at the 5′ end of the IRES is considered to be position 1. The relative location of the at least one RNA secondary structure and the sequence that is complementary to an 18S RNA may vary. For example, in some embodiments, the IRES comprises (i) at least one RNA secondary structure element and (ii) a sequence that is complementary to an 18S rRNA, and wherein the at least one RNA secondary structure is located 5′ to the sequence that is complementary to an 18S rRNA. In some embodiments, the IRES comprises (i) at least one RNA secondary structure element and (ii) a sequence that is complementary to an 18S rRNA, and wherein the at least one RNA secondary structure element is located 3′ to the sequence that is complementary to an 18S rRNA).

In some embodiments, the circular RNA may comprise one or more IRES RNA control elements. These elements may, in come embodiments, act as a conditional “off” switch. For example, the IRES RNA control element may be a miRNA binding site. miRNA binding to the circRNA may lead to degradation of the circRNA, destroying its activity.

### DNA Molecules and Host Cells

In some embodiments, the disclosure provides a DNA molecule comprising a nucleic acid sequence encoding any one of the recombinant circRNA molecules disclosed herein. Accordingly, described herein are DNA sequences that may be used to encode circular RNAs. In some embodiments, a DNA sequence encodes a circular RNA comprising an IRES. In some embodiments, a DNA sequence encodes a circular RNA comprising a protein-coding nucleic acid. In some embodiments, the DNA sequence encodes a circular RNA molecule; wherein the circular RNA molecule comprises a protein-coding nucleic acid sequence and an internal ribosome entry site (TRES) operably linked to the protein-coding nucleic acid sequence in a non-native configuration. In some embodiments, the DNA sequence encodes a protein coding-nucleic acid sequence, wherein the protein is a therapeutic protein.

The DNA sequences disclosed herein may, in some embodiments, comprise at least one non-coding functional sequence. For example, the non-coding functional sequence may be a microRNA (miRNA) sponge. A microRNA sponge may comprise a complementary binding site to a miRNA of interest. In some embodiments, a sponge's binding sites are specific to the miRNA seed region, which allows them to block a whole family of related miRNAs. In some embodiments, the miRNA sponge is selected from any one of the miRNA sponges shown in the table below.

| miRNA | Binding sites | Cell context |
|---|---|---|
| 92, empty | four bulged | rat cerebellar neuronal cultures |
| | sites | |
| miR-155, empty | eight bulged | B cell lymphoma cell line |
| | sites | |
| 15a, empty | two perfect sites | human prostate cell lines, xenograft |
| 113, empty | two perfect sites | mouse cardiac myocytes, heart |
| 326, empty | seven bulged | tail vein injection, TH cells |
| | sites | |
| 16, 20, 21, 30-5p, CXCR4 | four to nine | mammalian cell lines |
| | bulged sites | |
| 16, 20, CXCR4 | four to nine | mammalian cell lines |
| | bulged sites | |
| let-7, 22, 124, 125, 132, 143, | five to seven | rat hippocampal neuronal cultures |
| empty | bulged sites | |
| 183, CXCR4 | seven bulged | HEK293T cell line |
| | sites | |
| A.t. 156, 319, | one bulged site | transgenic Arabidopsis plants |
| empty | | |
| 16, CXCR4 | seven to nine | human multiple myeloma cell |
| | bulged sites | lines, xenograft |
| 16, 23a, 142-3p. | four perfect or | U937 cell line, mouse hematopoietic |
| 221, 223, | four | stem/progenitor cells, transplant |
| nonspecific | bulged sites | |
| KSHV miR-K1, | nine bulged | BC-3 B cell lymphoma cell line |
| CXCR4, empty | sites | |
| 21, 140-3p, | one to two | mammalian cell lines |
| 140-5p, empty | perfect sites | |
| 133, empty | three perfect | mouse cardiac myocytes |
| | sites | |
| 204, 211, empty | two perfect sites | ST2 cell line, osteogenic and |
| | | adipogenic differentiation |
| 96/182/183, empty | four bulged | mouse retina |
| | sites each | |
| let-7, 16, empty | six to nine | lung cancer cell line |
| | bulged sites | |
| D.m. 7, 8, 9a, 276a | 10 bulged sites | Transgenic fruitflies |
| 9, 10b, CXCR4 | eight bulged | 4T1 breast cancer cell line, xenograft |
| | sites | |
| herpesvirus miR-K12-7, K12-8, | six bulged sites | human B cell lines |
| BARTI-5p, BART4, empty | | |
| 144, 451, empty | four perfect | mouse hematopoietic stem/progenitor |
| | sites | cells, transplant |
| let-7, 29, 125, | 16 bulged sites | neural stem cells |
| let-7/125, | | |
| 128, empty | | |
| 21, empty | two perfect sites | mouse cardiocytes |
| 18a, 19b, 20a, | one perfect site | K562 cell line |
| nonspecific | | |
| 145/146, empty | eight to nine | mouse hematopoietic stem/progenitor |
| | bulged | cells, transplant |
| | sites each | |
| 31, CXCR4 | seven bulged | MCF7-Ras breast cancer cell line, |
| | sites | xenograft |

In some embodiments, the non-coding sequence may be an RNA binding protein site. RNA binding proteins and binding sites therefore are listed in numerous databases known to those of skill in the art, including RBPDB (rbpdb.ccbr.utoronto.ca). In some embodiments, the RNA binding protein comprises one or more RNA-binding domains, selected from RNA-binding domain (RBD, also known as RNP domain and RNA recognition motif, RRM), K-homology (KH) domain (type I and type II), RGG (Arg-Gly-Gly) box, Sm domain; DEAD/DEAH box, zinc finger (ZnF, mostly C-x8-X-x5-X-x3-H), double stranded RNA-binding domain (dsRBD), cold-shock domain; Pumilio/FBF (PUF or Pum-HD) domain, and the Piwi/Argonaute/Zwille (PAZ) domain.

In some embodiments, the DNA sequence comprises an aptamer. Aptamers are short, single-stranded DNA molecules that can selectively bind to a specific target. The target may be, for example, a protein, peptide, carbohydrate, small molecule, toxin, or a live cell. Some aptamers can bind DNA, RNA, self-aptamers or other non-self aptamers. Aptamers assume a variety of shapes due to their tendency to form helices and single-stranded loops. Illustrative DNA and RNA aptamers are listed in the Aptamer database (scicrunch.org/resources/Any/record/nlx_144509-1/SCR_001781/resolver?q=*&l=).

In some embodiments, the DNA sequence encodes a circular RNA molecule that comprises between about 200 nucleotides and about 10,000 nucleotides.

In some embodiments, the DNA sequence encodes a circular RNA molecule that comprises a spacer between the IRES and a start codon of the protein-coding nucleic acid sequence. The spacer may be of any length (e.g., 10 to 100 nucleotide, 10 to 90 nucleotides, 10 to 80 nucleotides, 10 to 70 nucleotides, 10 to 60 nucleotides, 10 to 50 nucleotides, 10 to 40 nucleotides, 10 to 30 nucleotides, 10 to 20 nucleotides, 20 to 100 nucleotides, 20 to 90 nucleotides, 20 to 80 nucleotides, 20 to 70 nucleotides, 20 to 60 nucleotides, 20 to 50 nucleotides, 20 to 40 nucleotides, 20 to 30 nucleotides, 30 to 100 nucleotides, 30 to 90 nucleotides, 30 to 80 nucleotides, 30 to 70 nucleotides, 30 to 60 nucleotides, 30 to 50 nucleotides, 30 to 40 nucleotides, 40 to 100 nucleotides, 40 to 90 nucleotides, 40 to 80 nucleotides, 40 to 70 nucleotides, 40 to 60 nucleotides, 40 to 50 nucleotides, 50 to 100 nucleotides, 50 to 90 nucleotides, 50 to 80 nucleotides, 50 to 70 nucleotides, 50 to 60 nucleotides, 60 to 100 nucleotides, 60 to 90 nucleotides, 60 to 80 nucleotides, 60 to 70 nucleotides, or 50 nucleotides). For example, in some embodiments, the length of the spacer is selected to optimize translation of the protein-coding nucleic acid sequence.

In some embodiments, the DNA sequence encodes a circular RNA molecule comprising an IRES that is configured to promote rolling circle translation. In some embodiments, the DNA sequence encodes a circular RNA comprising a protein-coding nucleic acid sequence that lacks a stop codon. In some embodiments, the DNA sequence encodes a circular RNA molecule comprising (i) an IRES that is configured to promote rolling circle translation, and (ii) a protein-coding nucleic acid sequence that lacks a stop codon.

The DNA sequences described herein may be comprised in one or more vectors. For example, in some embodiments, a viral vector comprises a DNA sequence encoding a circular RNA. The viral vector may be, for example, an adeno-associated virus (AAV) vector, an adenovirus vector, a retrovirus vector, a lentivirus vector, a vaccinia or a herpesvirus vector.

In some embodiments, the viral vector is an AAV. As used herein, the term “adeno-associated virus” (AAV), includes but is not limited to, AAVI, AAV2, AAV3 (inchiding types 3 A and 3B), AAV4, AAV5, AAV6, AAV7, AAV8, AAV9, AAVIO, AAV11, AAV12, avian AAV, bovine A AV, canine AAV, equine AAV, ovine AAV, and any other AAV now known or later discovered. In some embodiments, the AAV vector may be a modified form (i.e., a form comprising one or more amino acid modifications relative thereto) of one or more ofAAVI, AAV2, AAV3 (including types 3 A and 3B), AAV4, AAV5, AAV6, AAV7, AAV8, AAV9, AAVI0, AAVI 1, AAV12, avian AAV, bovine AAV, canine AAV, equine AAV, or ovine AAV. Various AAV serotypes and variants thereof are described, e.g., BERNARD N. FIELDS et al VIROLOGY, volume 2, chapter 69 (4th ed., Lippincott-Raven Publishers), A number of relatively new AAV serotypes and clades have been identified (see, e.g., Gao et al. (2004) J Virology 78:6381-6388; Moris et al (2004) Virology 33-:375~383). The genomic sequences of various serotypes of AAV, as well as the sequences of the native terminal repeats (TRs), Rep proteins, and capsid subunits are known in the art. Such sequences may be found in the literature or in public databases such as the GenBank© Database. See, e.g. , GenBank Accession Numbers NC_044927, NC 002077, NC_001401, NC_001729, NC 001863, NC 001829, NC_001862, NC_000883, NC 001701, NC 001510, NC_006152, NC_006261, AF063497, U89790, AF043303, AF028705, AF028704, J02275, JO 1901, J02275, X01457, AF288061, AH009962, AY028226, AY028223, NC 001358, NC 001540, AF513851, AF513852, AY530579; the disclosures of which are incorporated by reference herein for teaching parvovirus and AAV nucleic acid and amino acid sequences. See also, e.g., Srivistava et al. (1983) J Virology 45:555; Chiorini et al. (1998) J. Virology 71:6823; Chiorini et al (1999) J Virology 73:1309; Bantel-Schaal et al. (1999) J. Virology 73:939; Xiao et al. (1999) J. Virology 73:3994; Muramatsu et al. (1996) Virology 221:208; Shade et al. (1986) J Virol. 58:921; Gao et al. (2002) Proc. Nat. Acad. Sci. USA 99: 1 1854; Moris et al. (2004) Virology 33-:375-383; international patent publications WO 00/28061, WO 99/61601, WO 98/11244; and U.S. Pat. No. 6,156,303; the disclosures of which are incorporated by reference herein.

In some embodiments, a DNA sequence described herein is comprised in an AAV2 vector, or a variant thereof. In some embodiments, a DNA sequence described herein is comprised an AAV4 vector, or a variant thereof. In some embodiments, a DNA sequence described herein is comprised in an AAV8 vector, or a variant thereof. In some embodiments, a DNA sequence described herein is comprised in an AAV9 vector, or a variant thereof.

In some embodiments, a DNA sequence described herein is comprised in a viral-like particle (VLP). Viral like particles are molecules that closely resemble viruses, but are non-infectious because they contain little or no viral genetic material. They can be naturally occurring or synthesized through the individual expression of viral structural proteins, which can then self-assemble into a virus-lie structure. Combinations of structural capsid proteins from different viruses can be used to create VLPs. For example VLPs may be derived from the, AAVs, retrovirus, Flaviviridae, paramyoxoviridae, or bacteriophages. VLPs can be produced in multiple cell culture systems, including bacteria, mammalian cell lines, insect cell lines, yeast and plant cells.

In some embodiments, a DNA sequence described herein is comprised in a non-viral vector. The non-viral vector may be, for example, a plasmid comprises the DNA sequence. In some embodiments, the non-viral vector is a closed-ended DNA. A closed-ended DNA is a non-viral, capsid-free DNA vector with covalently closed ends (see, e.g., WO2019/169233). In some embodiments, a mini-intronic plasmid vector comprises a DNA sequence described herein. Mini-intronic plasmids are expression systems that contain a bacterial replication origin and selectable marker maintaining the juxtaposition of the 5′ and the 3′ ends of transgene expression cassette as in a minicircle (see, e.g., Lu, J., et al., Mol Ther (2013) 21(5) 954-963).

In some embodiments, a DNA sequence described herein is comprised in a lipid nanoparticle. Lipid nanoparticles (or LNPs) are submicron-sized lipid emulsions, and may offer one or more of the following advantages: (i) control and/or targeted drug release, (ii) high stability, (iii) biodegradability of the lipids used, (iv) avoid organic solvents, (v) easy to scale-up and sterilize, (vi) less expensive than polymeric/surfactant based carriers, (vii) easier to validate and gain regulatory approval. In some embodiments, the lipid nanoparticles range in diameter between about 10 and about 1000 nm.

In some embodiments, a DNA sequence encodes a circular RNA molecule, wherein the circular RNA molecule comprises a protein-coding nucleic acid sequence and an internal ribosome entry site (IRES) operably linked to the protein-coding nucleic acid sequence in a non-native configuration wherein the IRES comprises: at least one RNA secondary structure; and a sequence that is complementary to an 18S ribosomal RNA (rRNA).

In some embodiments, a DNA sequence encodes a circular RNA molecule, wherein the circular RNA molecule comprises a protein-coding nucleic acid sequence and an internal ribosome entry site (IRES) operably linked to the protein-coding nucleic acid sequence in a non-native configuration wherein the IRES comprises: at least one RNA secondary structure element; and a sequence that is complementary to an 18S ribosomal RNA (rRNA); wherein the IRES has a minimum free energy (MFE) of less than −18.9 kJ/mol and a melting temperature of at least 35.0° C.; and wherein the RNA secondary structure element is formed from the nucleotides at about position 40 to about position 60 of the IRES, wherein the first nucleic acid at the 5′ end of the IRES is considered to be position 1.

In some embodiments, a DNA sequence comprises a nucleic acid sequence encoding a circular RNA molecule; wherein the circular RNA molecule comprises a protein-coding nucleic acid sequence and an internal ribosome entry site (IRES) operably linked to the protein-coding nucleic acid sequence in a non-native configuration; wherein the IRES is encoded by any one of the nucleic acid sequences listed in SEQ ID NOs: 138-17338, or a nucleic acid sequence that is at least 90% or at least 95% identical thereto.

Also provided herein are cells comprising a recombinant circRNA molecule, a DNA molecule, or a vector described herein. Any prokaryotic or eukaryotic cell that can be contacted with and stably maintain the recombinant circRNA molecule, DNA molecule encoding the recombinant circRNA molecule, or vector comprising the recombinant circRNA molecule may be used in the context of the present disclosure. Examples of prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. In some embodiments, the host cell is a eukaryotic cell. Suitable eukaryotic cells are known in the art and include, for example, yeast cells, insect cells, and mammalian cells. Examples of yeast cells include those from the genera Hansenula, Kluyveromyces, Pichia, Rhinosporidium, Saccharomyces, and Schizosaccharomyces. Suitable insect cells include Sf-9 and HIS cells (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993).

In some embodiments, the cell is a mammalian cell. A number of mammalian cells are known in the art, many of which are available from the American Type Culture Collection (ATCC, Manassas, Va.). Examples of mammalian cells include, but are not limited to, HeLa cells, HepG2 cells, Chinese hamster ovary cells (CHO) (e.g., ATCC No. CCL61), CHO DHFR-cells (Urlaub et al., Proc. Natl. Acad. Sci. USA, 97: 4216-4220 (1980)), human embryonic kidney (HEK) 293 or 293T cells (e.g., ATCC No. CRL1573), and 3T3 cells (e.g., ATCC No. CCL92). Other mammalian cell lines are the monkey COS-1 (e.g., ATCC No. CRL1650) and COS-7 cell lines (e.g., ATCC No. CRL1651), as well as the CV-1 cell line (e.g., ATCC No. CCL70). Further exemplary mammalian host cells include primate cell lines and rodent cell lines, including transformed cell lines. Normal diploid cells, cell strains derived from in vitro culture of primary tissue, as well as primary explants also are suitable. Other mammalian cell lines include, but are not limited to, mouse neuroblastoma N2A cells, HeLa, mouse L-929 cells, and BHK or HaK hamster cell lines, all of which are available from the American Type Culture Collection (ATCC; Manassas, VA). Methods for selecting mammalian cells and methods for transformation, culture, amplification, screening, and purification of such cells are well known in the art (see, e.g., Ausubel et al., supra). In some embodiments, the mammalian cell is a human cell.

### Method of Producing a Protein

The disclosure further provides a method of producing a protein in a cell, which comprises contacting a cell with the above-described recombinant circular RNA molecule, the above-described DNA molecule comprising a nucleic acid sequence encoding the recombinant circRNA molecule, or a vector comprising the recombinant circRNA molecule under conditions whereby the protein-coding nucleic acid sequence is translated and the protein is produced in the cell.

In some embodiments, a method of producing a protein in a cell comprises contacting a cell with a DNA sequence described herein, or a vector comprising the DNA sequence, under conditions whereby the protein-coding nucleic acid sequence is translated and the protein is produced in the cell. Also provided is a protein produced by the disclosed methods.

In some embodiments, production of the protein is tissue-specific. For example, the protein may be selectively produced in one or more of the following tissues: muscle, liver, kidney, brain, lung, skin, pancreas, blood, or heart.

In some embodiments, the protein is expressed recursively in the cell.

In some embodiments, the half-life of the circular RNA in the cell is about ito about 7 days. For example, the half-life of the circular RNA may be about 1, about 2, about 3, about 4, about 5, about 6, about 7, or more days.

In some embodiments, the protein is produced in the cell for at least about 10%, at least about 20%, or at least about 30% longer than if the protein-coding nucleic acid sequence is provided to the cell using a viral vector encoding a linear RNA or as a linear RNA.

In some embodiments, the protein is produced in the cell at a level that is at least about 10%, at least about 20%, or at least about 30% higher than if the protein-coding nucleic acid sequence is provided to the cell using a viral vector or as a linear RNA.

Use of the IRES sequences described herein to express a protein from a circular RNA may, in some embodiments, allow for continued expression of a protein from the circular RNA in a cell even under stress conditions. In response to one or more stress conditions, production of proteins from linear RNA is often suppressed. Accordingly, in some embodiments, circRNA can be used as an alternative for production of proteins from linear RNAs during stress conditions. In some embodiments, a protein expressed from a circular RNA in a cell is expressed under one or more stress conditions. In some embodiments, expression of a protein from a circular RNA in a cell is not substantially disrupted when the cell is exposed to one or more stress conditions. For example, exposure of the cell to one or more stress conditions may change expression of a protein from a circular RNA by less than 15%, less than 10%, less than 5%, less than 3%, less than 1%, or less than 0.5%. In some embodiments, a protein expressed from a circular RNA is expressed at a level under one or more stress conditions that is substantially the same as the level expressed in the same cell in the absence of the one or more stress conditions. In some embodiments, the level of expression of a protein from a circular RNA in a cell is at least about 70%, at least about 75%, at least about 80%, at least about 85%, at least about 90%, at least about 95%, or at least about 99%, relative to the level of expression in the absence of the one or more stress conditions. A non-limiting list of conditions which may cause cellular stress include changes in temperature (including exposure to extreme temperatures and/or heat shock), exposure to toxins (including viral or bacterial toxins, heavy metals, etc.), exposure to electromagnetic radiation, mechanical damage, viral infection, etc.

In some embodiments, the circRNAs described herein (including components thereof, such as the IRES sequences) facilitate cap-independent translation activity from the circRNA. Canonical translation via a cap-independent mechanism may be reduced in some human diseases. Accordingly, the use of circRNAs to express proteins may be particularly helpful for treating such diseases. In some embodiments, use of the circRNAs described herein facilitates cap-independent translation activity from the circRNA under conditions wherein cap-dependent translation is reduced or turned-off in a cell.

As discussed above, translation of the protein-coding nucleic acid sequence may occur in an infinite loop (i.e., recursively) when the IRES is in-frame with the protein-coding nucleic acid sequence and the protein-coding sequence lacks a stop codon. Thus, in some embodiments, the method of producing a protein in a cell produces a concatenated protein.

Any prokaryotic or eukaryotic host cell described herein may be contacted with the recombinant circRNA molecule or a vector comprising the circRNA molecule. The host cell may be a mammalian cell, such as a human cell. In some embodiments, the cell is in vivo. In some embodiments, the cell is in vitro. In some embodiments, the cell is ex vivo. In some embodiments, the cell is in a mammal, such as a human.

In some embodiments, regardless of cell type chosen, 5′ cap-dependent translation is impaired in the cell (e.g., decreased, reduced, inhibited, or completely obliterated). In some embodiments, there is no substantial 5′ cap-dependent translation in the cell.

The circRNAs described herein may also be produced in vitro, such as by in vitro transcription or other cell-free transcription system. Typical in vitro transcription protocols comprise providing (i) a purified DNA template, wherein the DNA template encodes a circular RNA, (ii) ribonucleotide triphosphates, (iii) a buffer system that includes DTT and magnesium ions, and (iv) an appropriate phage RNA polymerase. The DNA template may comprise, for example, a plasmid construct engineered by cloning, a cDNA template generated by first- and second-strand synthesis from an RNA precursor (e.g., aRNA amplification), or a linear template generated by PCR or by annealing chemically synthesized oligonucleotides. These components are then combined, and incubated under conditions which allows the RNA polymerase to transcribe the DNA to RNA, typically a linear RNA. Commercial kits are available for performing in vitro transcription, such as the Invitrogen MAXIscript© or MEGAscript© kits. In some embodiments, a polyA tail may be added to an RNA produced using in vitro transcription.

Linear RNAs produced in vitro may be circularized using one or more of the following exemplary methods. For example, linear RNAs produced in vitro may be circularized according to chemical methods, using a condensing agent such as cyanogen bromide. In some embodiments, linear RNAs produced in vitro may be circularized using an enzymatic method. For example, the linear RNAs may be circularized using RNA or DNA ligases (e.g., T4 RNA ligase I or II). Alternatively, the linear RNAs may be circularized using ribozymatic methods, such as methods which employ self-splicing introns.

In some embodiments, a protein is produced from a circular RNA in a cell free system. The cell-free system may comprise, for example, all factors required for transcribing circular RNA from DNA, circularizing the RNA, and translating the protein from therefrom. In some embodiments, the circular RNA is more stable than a linear RNA in a cell-free system, which allows for increased expression of a protein from the circular RNA.

In some embodiments, a method for producing a protein comprises contacting a circular RNA with a cell-free extract comprising protein translation initiation factors (e.g., eIF1, eIF2, eIF3, eIF5, eIF6), under conditions wherein the protein is expressed. In some embodiments, a method for producing a protein comprises: (i) providing a linear RNA encoding a protein of interest, (ii) circularizing the RNA, (iii) contacting the circular RNA with a cell-free extract comprising protein translation initiation factors, under conditions wherein the protein is expressed.

In some embodiments, a method for producing a protein comprises contacting a linear RNA with a cell-free extract comprising protein translation initiation factors, under conditions wherein the RNA is circularized and the protein is expressed. In some embodiments, the linear RNA may comprise self-splicing introns.

In some embodiments, a method for producing a protein comprises contacting a DNA with a cell-free extract comprising protein translation initiation factors, under conditions wherein a linear RNA is expressed, the linear RNA is circularized, and the protein is expressed. In some embodiments, the DNA may encode may comprise self-splicing introns.

The recombinant circular RNA molecule, a DNA molecule encoding same, or vectors comprising same, may be introduced into a cell by any method, including, for example, by transfection, transformation, or transduction. The terms “transfection,” “transformation,” and ¹transduction” are used interchangeably herein and refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); strontium phosphate DNA co-precipitation (Brash et al., Mol. Cell. Biol., 7: 2031-2034 (1987); and magnetic nanoparticle-based gene delivery (Dobson, J., Gene Ther, 13 (4): 283-7 (2006)).

Naked RNA, DNA molecules encoding circular RNA molecules, or vectors comprising the circular RNAs or DNAs encoding circular RNAs may be administered to cells in the form of a composition. In some embodiments, the composition comprises a pharmaceutically acceptable carrier. The choice of carrier will be determined in part by the particular circular RNA molecule, DNA sequence, or vector and type of cell (or cells) into which the circular RNA molecule, DNA sequence, or vector is introduced. Accordingly, a variety of formulations of the composition are possible. For example, the composition may contain preservatives, such as, for example, methylparaben, propylparaben, sodium benzoate, and benzalkonium chloride. A mixture of two or more preservatives optionally may be used. In addition, buffering agents may be used in the composition. Suitable buffering agents include, for example, citric acid, sodium citrate, phosphoric acid, potassium phosphate, and various other acids and salts. A mixture of two or more buffering agents optionally may be used. Methods for preparing compositions for pharmaceutical use are known to those skilled in the art and are described in more detail in, for example, Remington: The Science and Practice of Pharmacy, Lippincott Williams & Wilkins; 21st ed. (May 1, 2005).

In some embodiments, the composition containing the recombinant circular RNA molecule, DNA sequence, or vector, can be formulated as an inclusion complex, such as cyclodextrin inclusion complex, or as a liposome. Liposomes can be used to target host cells or to increase the half-life of the circular RNA molecule. Methods for preparing liposome delivery systems are described in, for example, Szoka et al., Ann. Rev. Biophys. Bioeng., 9: 467 (1980), and U.S. Pat. Nos. 4,235,871; 4,501,728; 4,837,028; and 5,019,369. The recombinant circRNA molecule may also be formulated as a nanoparticle.

A host cell can be contacted in vivo or in vitro with a recombinant circRNA molecule, a DNA sequence, or a vector, or compositions containing any of the foregoing. The term “in vivo” refers to a method that is conducted within living organisms in their normal, intact state, while an “in vitro” method is conducted using components of an organism that have been isolated from its usual biological context. When the method is conducted in vivo, in some embodiments the production of the protein is tissue-specific. By “tissue-specific” is meant that the protein is produced in only a subset of tissue types within an organism, or is produced at higher levels in a subset of tissue types relative to the baseline expression across all tissue types. The protein may be produced in any tissue type, such as, for example, tissues of muscle, liver, kidney, brain, lung, skin, pancreas, blood, or heart.

Various embodiments of this invention are described herein, including the best mode known to the inventors for carrying out the invention. Variations of these embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

### EXAMPLES

The following examples further illustrate the invention but, of course, should not be construed as in any way limiting its scope.

### Example 1: Viral IRES Screen

The following example describes the development of a high-throughput screen to systematically identify and quantify viral IRES RNA sequences that can direct circRNA translation.

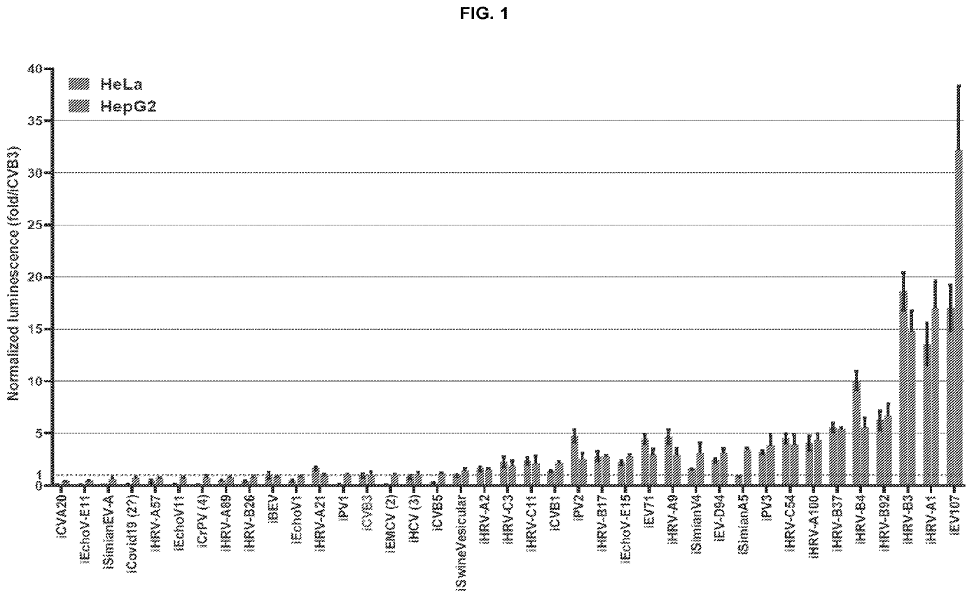

A large set of IRESs (see Table 7) representing a diverse range of viral species were operably linked to a NanoLuciferase transgene (cloned from Promega vector #N1441) in a circular RNA format. The IRESs were selected to sample a large phylogenetic range of mammalian viral IRESs with well-annotated 5′UTR regions provided on NCBI Virus in order to better understand particular viral groups whose IRESs may drive strong translation in circRNAs comprising a luciferase transgene. These synthesized circRNAs were tested by transfection into HeLa and HepG2 cell lines. Protein production of NanoLuciferase was measured via luciferase assay, normalized to constitutive expression of Firefly Luciferase. Normalized fold/CVB3 IRES expression mean±SEM (standard error of the mean) are shown in FIG. 1.

As a result of this screen, type 1 IRESs and in particular human rhinovirus (HRV) IRESs were identified as strong drivers of circular RNA (circRNA) translation from among a diverse panel of RESs.

### Example 2: Rapid IRES Screening with Cell-Free Lysate

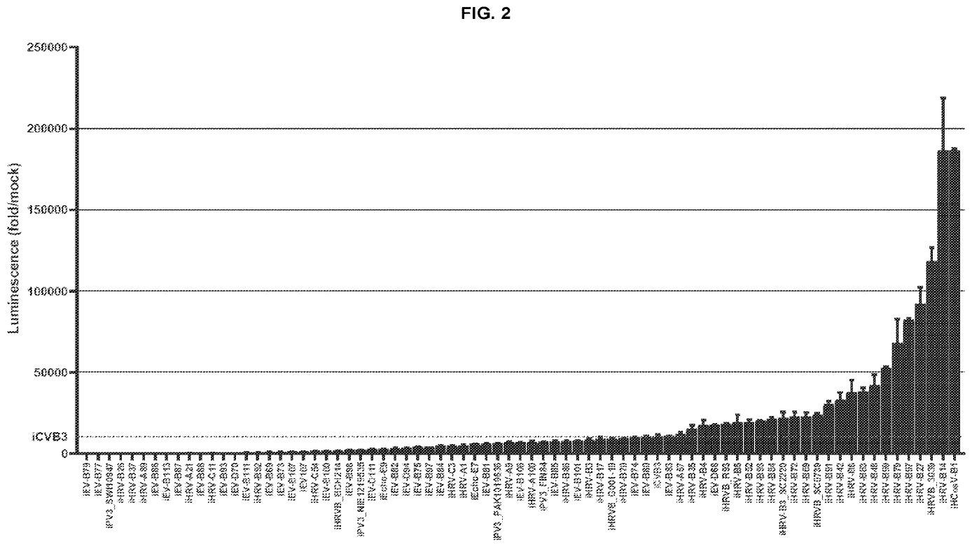

Because HRV IRESs were identified as strong drivers of circRNA translation in the cell-based screen of Example 1, a focused screen of every sequenced and publicly-available rhinovirus type B (HRV-B) and enterovirus B (EV) IRESs was performed in a cell-free assay. A number of other IRESs, namely CVB3, served as benchmarking controls. Plasmids encoding NanoLuciferase expression driven by the IRESs were cloned and served as template for in vitro transcription reactions. circRNAs produced by these reactions served as template for in vitro translation reactions with HeLa lysate. Mean luminescence fold/mock ±SEM are shown in FIG. 2.

This screen identified stronger human rhinovirus (HRV) IRESs for circRNA translation.

### Example 3: Expanded Viral IRES Screen in Diverse Cell Lines

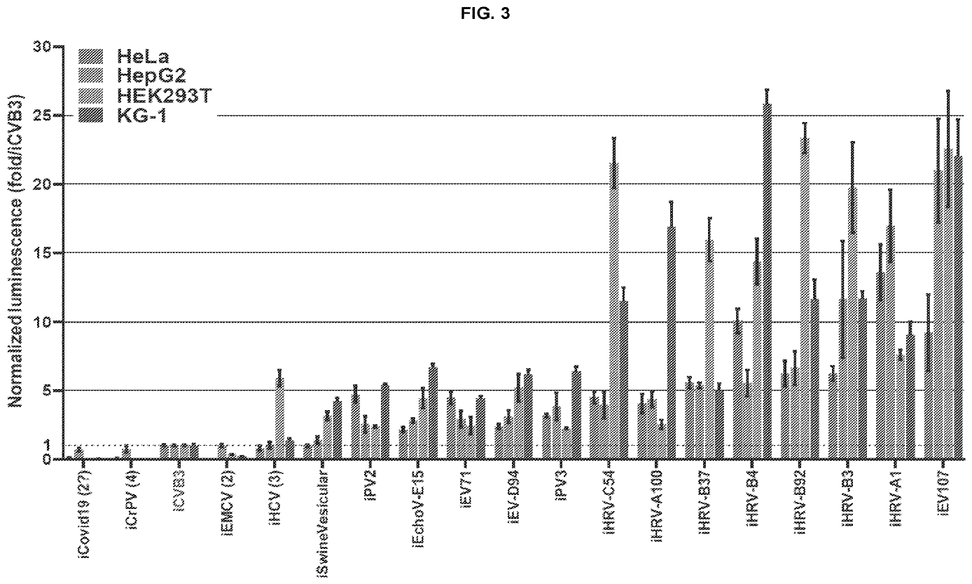

To test the function of various IRES sequences in different cell lines, a number of circRNAs comprising IRESs operably linked to NanoLuciferase were synthesized and tested by transfection into HeLa (cervical cancer), HepG2 (hepatocellular carcinoma), HEK293T (human embryonic kidney), and KG-1 (macrophage) cell lines. Protein production of NanoLuciferase transgene from the circRNA was measured via luciferase assay, normalized to constitutive expression of Firefly Luciferase. Normalized fold/CVB3 IRES expression mean±SEM are shown in FIG. 3. In this study, human rhinovirus (HRV) IRESs, particularly HRV-A1, HRV-B3, HRV-B92, and HRV-B4, were identified to be the strongest drivers of circRNA translation in a diverse set of cell lines.

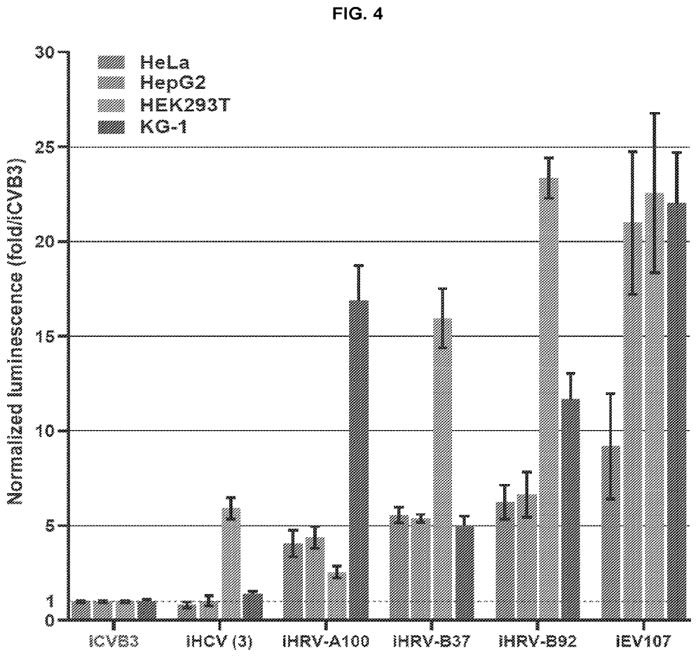

As shown in FIG. 4, focusing in on data from this large-scale IRES testing reveals that some IRESs have cell-type expression specificities. Hepatitis C (HCV) IRES had strong expression in HEK293T cells, as did human rhinovirus B (HRVB) 37 and 92 IRESs. HRV-A100 had strong expression in KG-1 cells exclusively. Enterovirus (EV) 107 had strong expression in all the tested cells, except for HeLa cells.

### Example 4: Rational Structural RNA Engineering of iCVB3 IRES

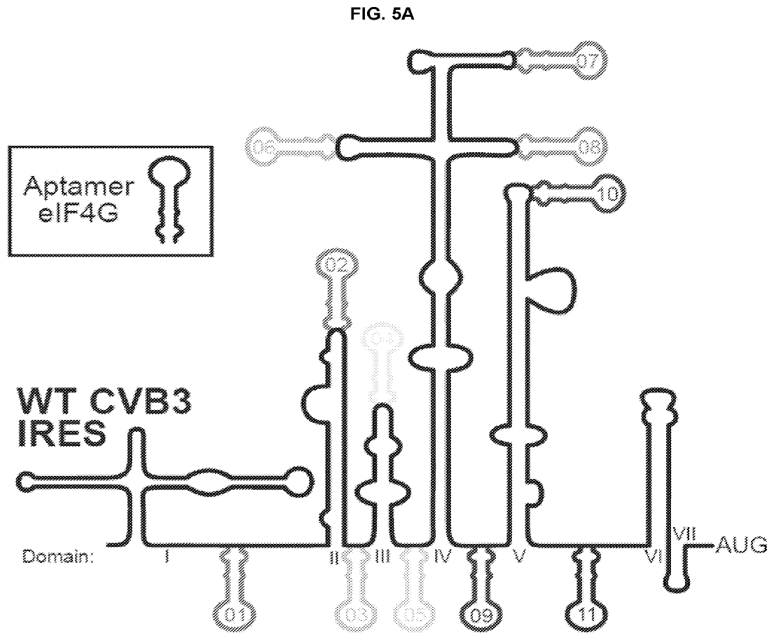

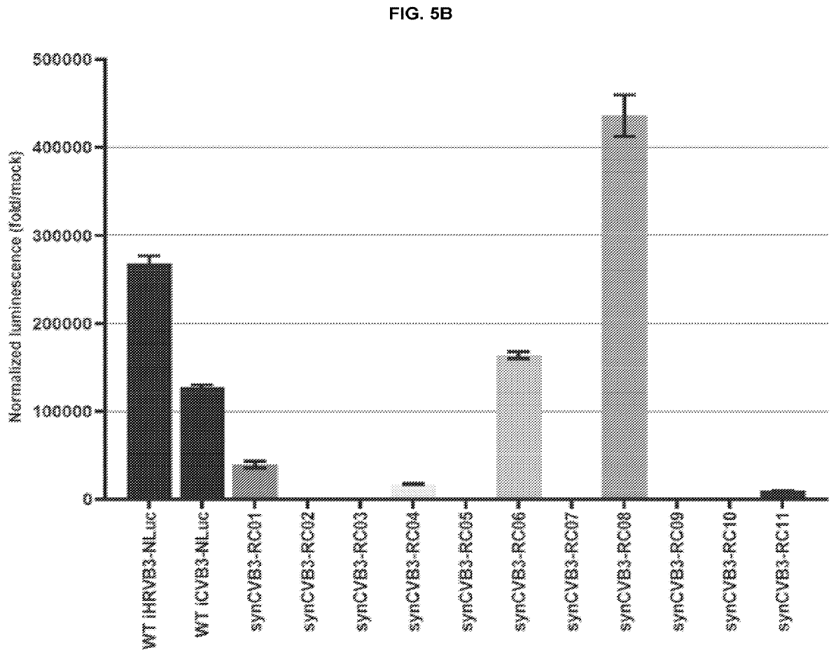

To determine whether rational structural engineering of the aptamers identified in the screens described above could further improve protein translation from a circRNA, an eIF4G-recruiting aptamer (FIG. 5A) was inserted at various locations within the CVB3 IRES (see SEQ ID NO: 101-125). The resulting synthetic IRES constructs were operably linked to a NanoLuciferase transgene and synthesized into circRNA format. Protein expression from the circRNAs was assayed after transfection thereof into HeLa cells. Specifically, NanoLuciferase expression from the circRNA was assayed and normalized to mock-transfected cells.

As shown in FIG. 5B, wild-type iHRV-B3 IRES was a strong IRES, followed by wild-type iCVB3 IRES and the synthetic IRES variants (RC01-11). Notably, aptamer eIF4G insertion into position 6 and 8 (i.e., in the proximal loop of domain IV of the iCVB3 IRES, wherein “proximal” is relative to Domain 5 of the natural eiF4G binding site of the IRES, see FIG. 5A) improved translation strength. Insertion in position 8 improved CVB3's translation strength to beyond that of HRV-B3.

Taken together, this data indicates that rational structural RNA engineering with eIF4G-recruiting aptamer insertions into iCVB3 IRES improves translation activity.

### Example 5: Rational Structural Engineering of the iHRV-B3 IREs

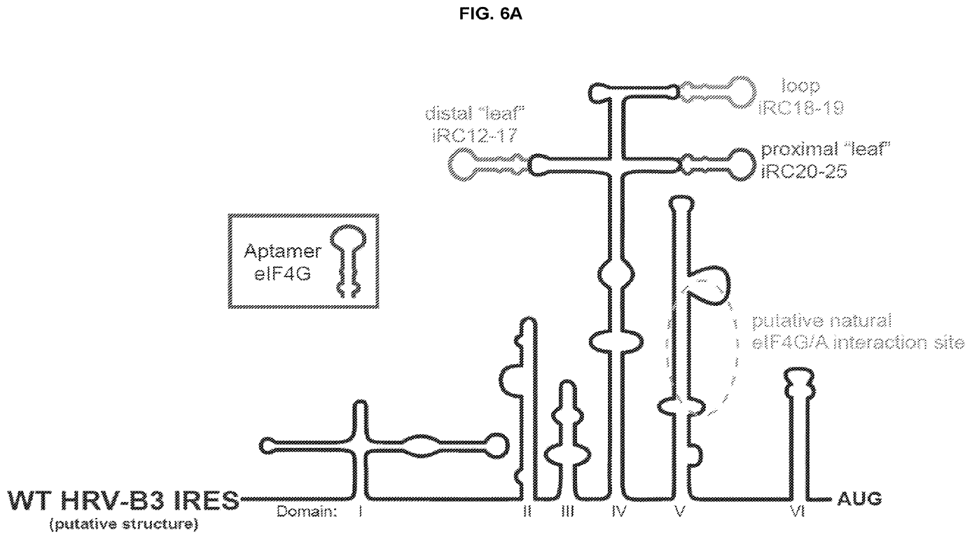

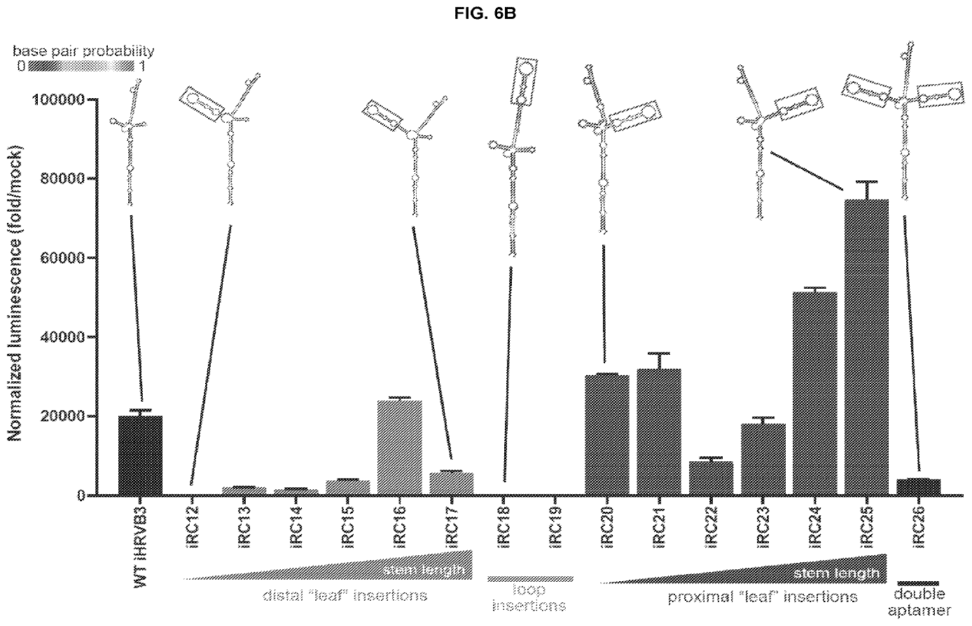

An eIF4G-recruiting aptamer was inserted at various locations within the iHRV-B3 IRES to generate synthetic IRESs (FIG. 6A). Although iHRV-B3's IRES structure is uncharacterized, alignment of sequence between iHRVB3 and iCVB3 IRESs was sufficient to identify key structural elements. Stem length was varied by truncating or lengthening the dsRNA stem region connecting the eIF4G aptamer to the rest of the IRES, and RNAfold predicted structures are shown in FIG. 6B. The resulting constructs IRES constructs were operably linked to a NanoLuciferase transgene, synthesized into circRNA format, and assayed by transfection into HeLa cells. NanoLuciferase expression was assayed and normalized to constitutive expression of Firefly Luciferase. Results are shown in FIG. 6B.

Taken together, this data shows that insertion of eIF4G-recruiting aptamer into HRV-B3 IRES domain IV at the proximal leaf position and further RNA structural optimization at this site engineered a synthetic IRES with improved translation.

### Example 6: a Full-Length Viral IRES is Important for Strong Translation

Viral IRESs are diverse and highly-structured RNA regions found primarily in viral 5′ UTRs that promote cap-independent translation (Kieft 2008 Trends Biochem. Sci. 33, 274-283, Filbin 2009 Curr. Opin. Struct. Biol. 19, 267-276, Martinez-Salas 2018 Front. Microbiol. 8, 2629). Because iCVB3 is nearly 750 bp it was determined if it was possible to truncate an IRES while retaining circRNA translation. A previous structure map of iCVB3 divided the sequence into seven domains (Bailey 2007 J. Virol. 81, 650-668), beginning with domain I containing a cloverleaf structure thought to be critical for viral replication (Murray 2004 J. Virol. 65, 5886-5894). Domains II-V have also been reported to interact with multiple IRES trans-activating factors (ITAFs) (de Breyne 2009 Proc. Natl. Acad. Sci. 106, 9197-9202, Souii 2013 Mol. Biotechnol. 2013 552 55, 179-202, Sweeney 2013 EMBO J. 33, 76-92), while domain VI hosts an AUG upstream of the true translation initiation site that recruits the 43S ribosomal preinitiation complex (Nicholson 1991 J. Virol. 65, 5886-5894, Yang 2003 Virology 305, 31-43, Sweeny 2013; supra).

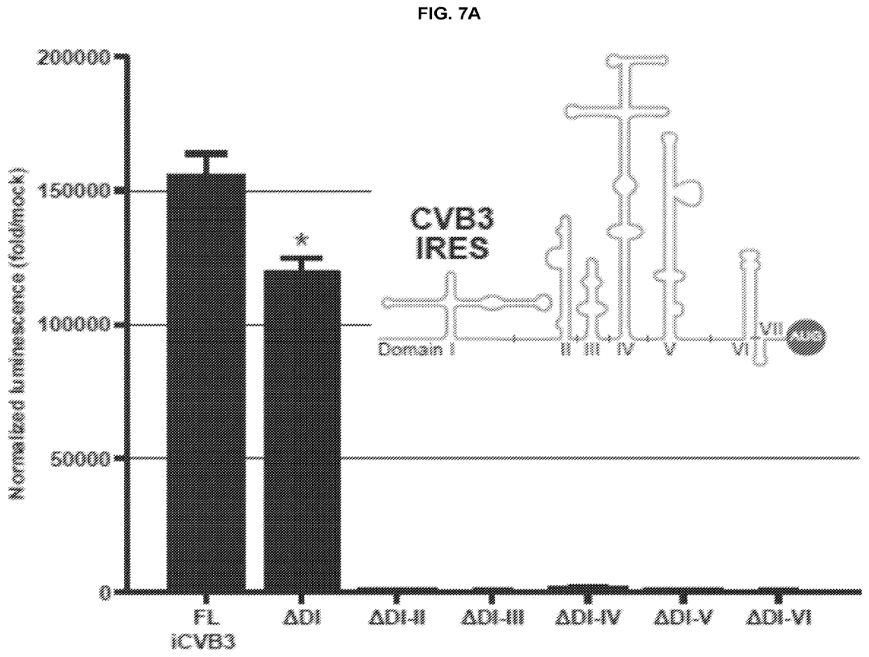

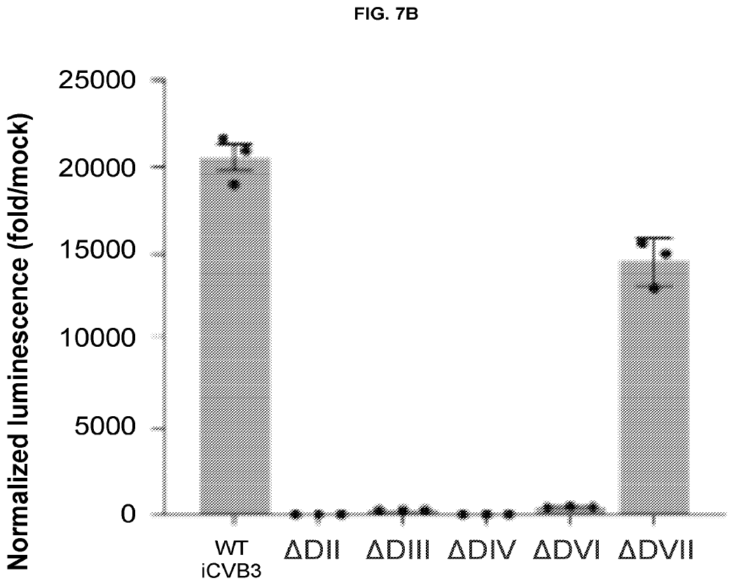

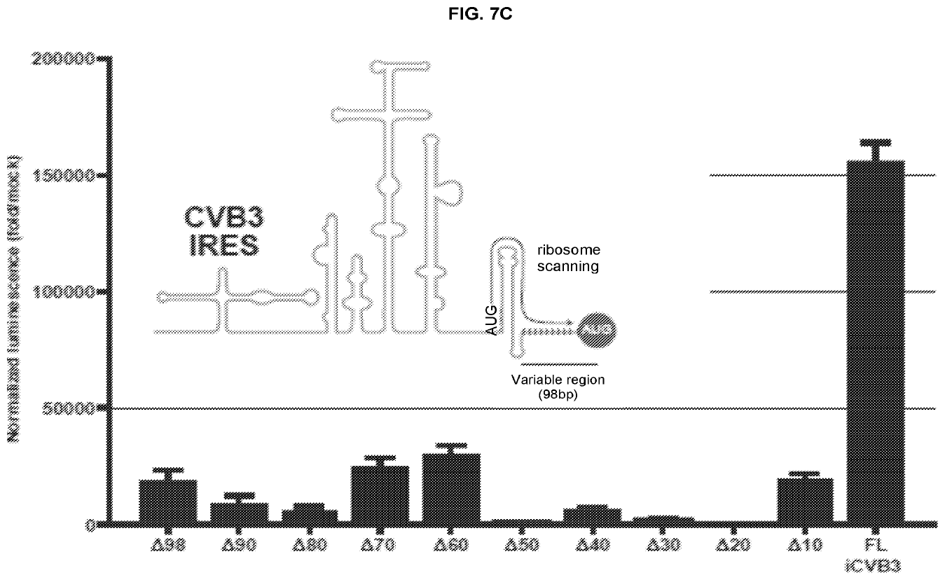

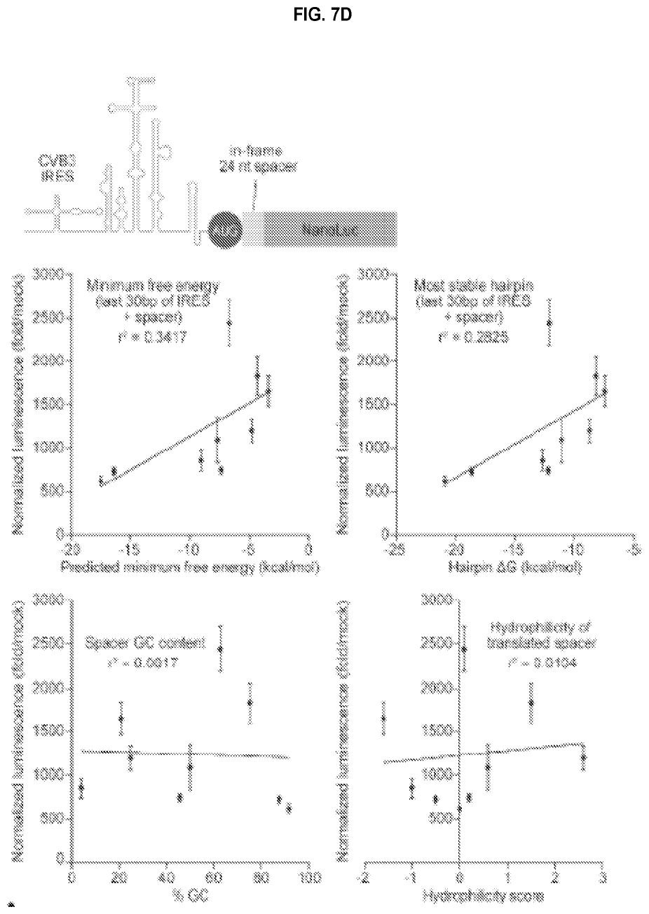

IRES domain truncations starting from the 5′ end of iCVB3 were performed, choosing truncations at boundaries where there was little known secondary structure base pairing. Compared to the full-length IRES, deletion of domain I significantly cut circRNA translation by 25%, and further deletions completely eliminated translational activity (FIG. 7A-B). Successive truncations of iCVB3 from the 3′ end were then performed. This region between domain VII and the start codon is highly variable in both sequence and length among different picornavirus IRESs, so it was hypothesized that it would be amenable to shortening. 3′ deletion of as few as ten terminal nucleotides from this region nearly ablated circRNA translation (FIG. 7C). Together, these data show that a full-length IRES is necessary for strong circRNA translation.

### Example 7: IRES-Coding Sequence Junction Secondary Structure Dictates Translation Strength

Coding sequence-specific factors that influence translation initiation in circRNAs were investigated by synthesizing circRNAs with nine different 24 bp N-terminal leader sequences in frame between the AUG start codon and the NanoLuc reporter (FIG. 7D). Various features of these leader sequences—secondary structure, GC content, and translated hydrophilicity—were compared against the resulting NanoLuc reporter strength. Indicators of secondary structure stability, such as predicted minimum free energy and free energy change for the most stable hairpin, were most correlated with NanoLuc translation (Gruber 2008 Nucleic Acids Res. 36, W70-W74), with 34.2% and 28.3% of variation in translation strength explained by those factors, respectively. On the other hand, the GC content of the N-terminal leader and hydrophilicity of its encoded peptide were not predictive of translation efficiency. These findings indicate that in silico optimization of base-pairing interactions between an IRES and coding sequence can yield additional benefits for circRNA translation.

### Example 8: Vector Topology and Spacer Requirements for circRNA Translation

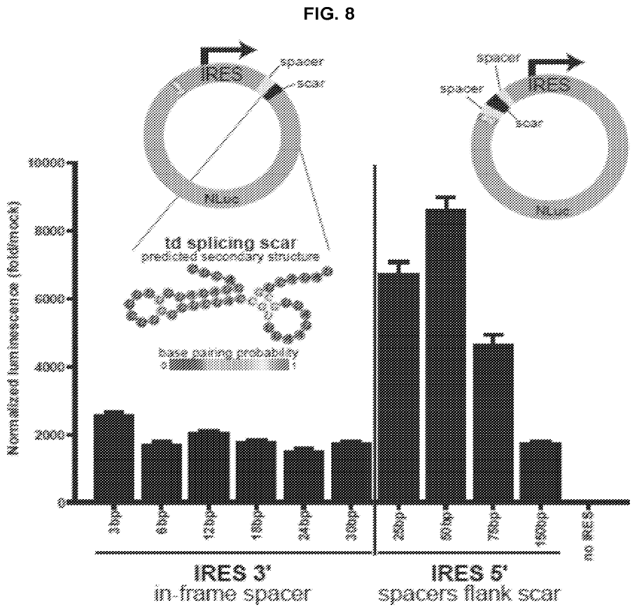

Principles behind circRNA vector topology that are needed for strong translation were investigated. First, circRNAs with the IRES downstream, or 3′, of the reporter NanoLuc gene, maintaining the reading frame through the residual scar formed by the self-splicing reaction of the T4 td intron, were synthesized. In this orientation, translation through the splicing scar is unavoidable. Hypothesizing that the highly structured scar sequence may obfuscate the translation start site, circRNA variants with in-frame spacers of varying lengths between the translation start and the splicing scar were synthesized. The peptides encoded by these spacers reflected consensus viral leader peptide sequences from the rhinovirus family. Testing the expression of these circRNAs indicated that increasing the spacer length was non-beneficial for translation, and that the ribosome was unaffected by the td splicing scar's secondary structure (FIG. 8).

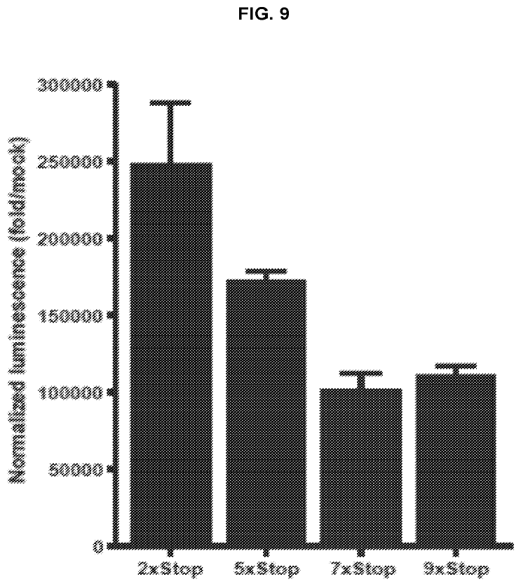

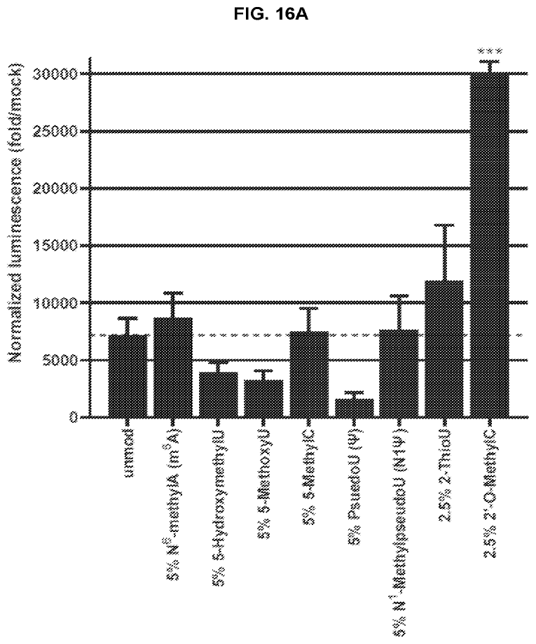

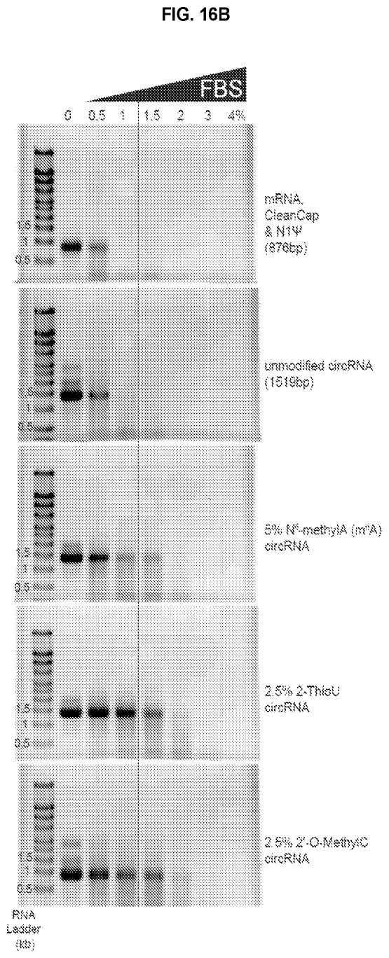

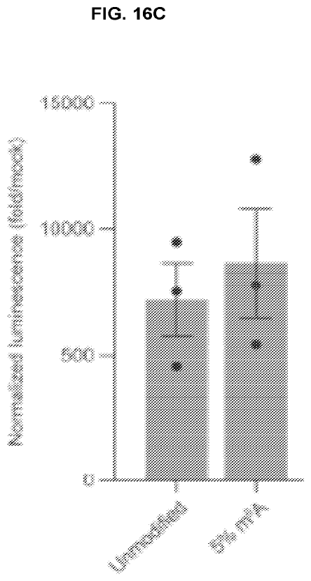

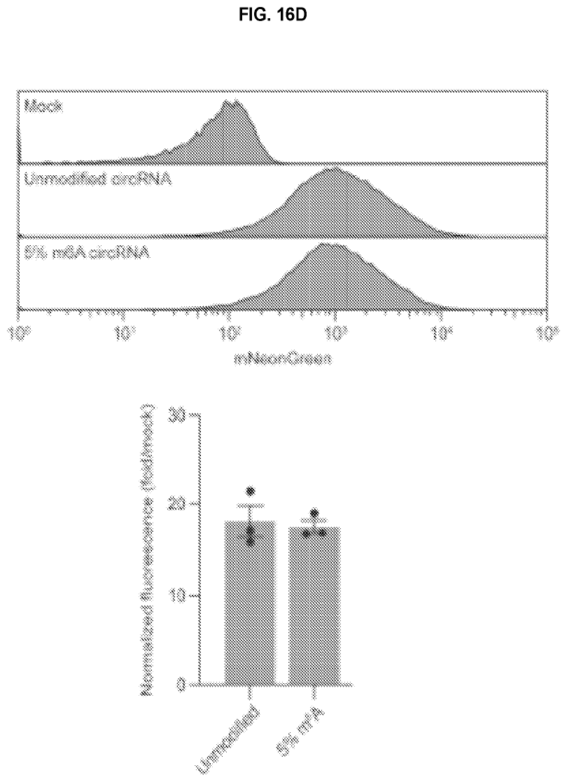

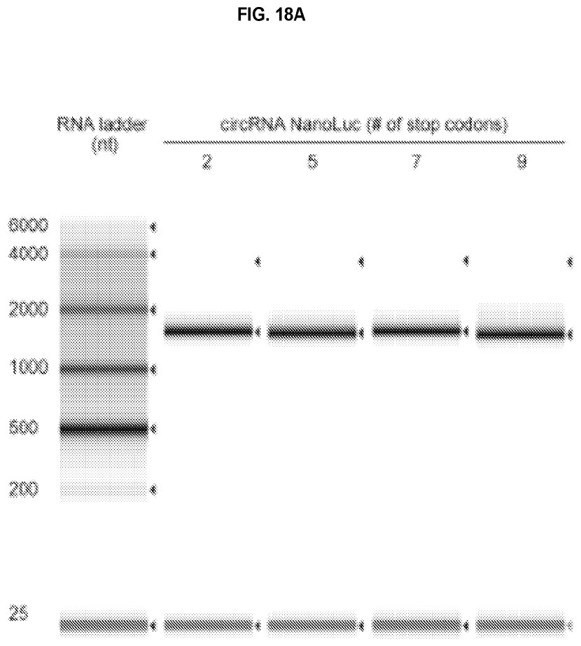

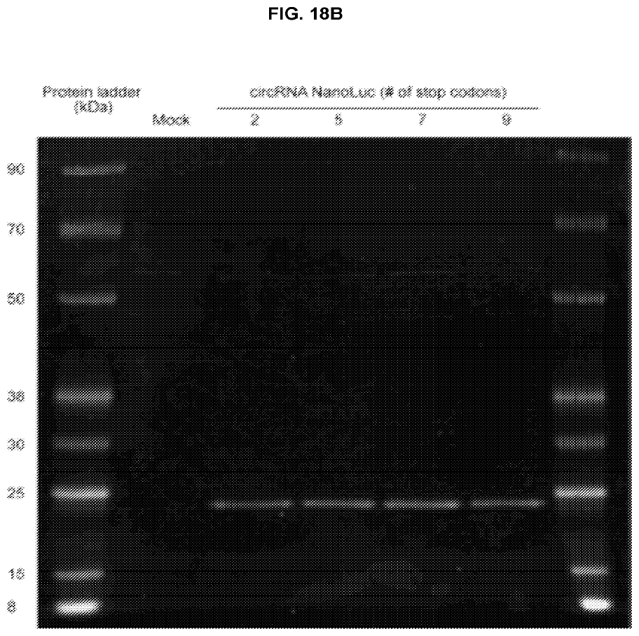

The topology of the circRNA vector was reversed, placing the IRES immediately upstream of the NanoLuc gene. When the IRES is 3′ to the NanoLuc reporter, translation through the td splicing scar is unavoidable. The predicted secondary structure of this scar is shown in FIG. 8. Flanking this translation cassette, adding spacers derived from random 50% GC content sequences of varying lengths in the 5′ and 3′ untranslated regions (UTRs) of the circRNA was tested. When assayed for NanoLuc expression, it was found that circRNAs with spacers 50 bp in length yielded the strongest translation (FIG. 8 and FIG. 16D). It was also tested whether the number of stop codons following the coding sequence affected circRNA expression and found that adding more than two stop codons reduced translation strength (FIG. 9) but did not affect the size of the encoded protein (FIG. 16D, FIG. 18A and FIG. 18B). The results indicate that IRES-mediated translation of circRNAs can occur readily through an intron splicing scar, though with reduced efficiency compared to the IRES being directly upstream of a gene. Furthermore, translation of circRNAs can be improved by the addition of 50 bp spacers separating the IRES and gene of interest from the splicing scar.

### Example 9: Synthetic IRES Engineering with an eIF4G-Binding Aptamer

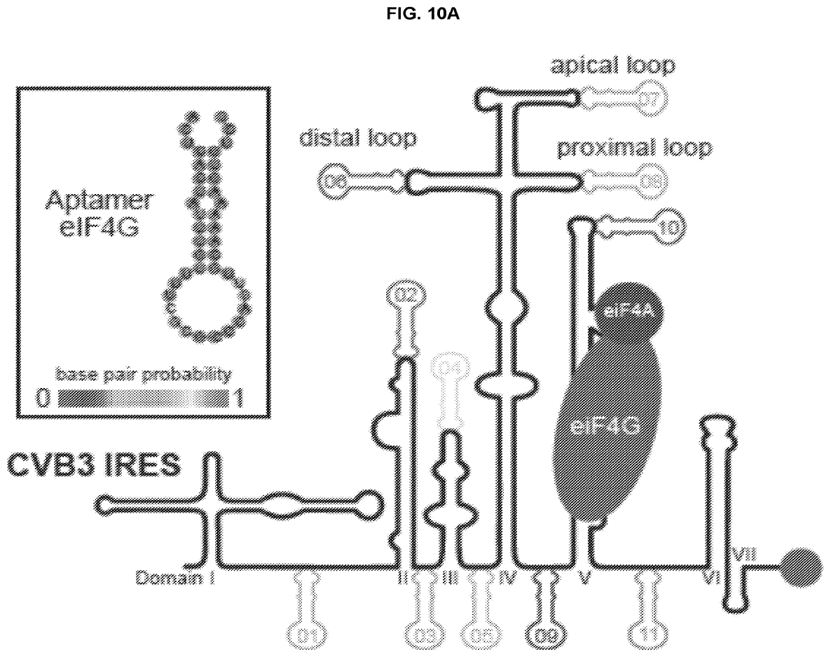

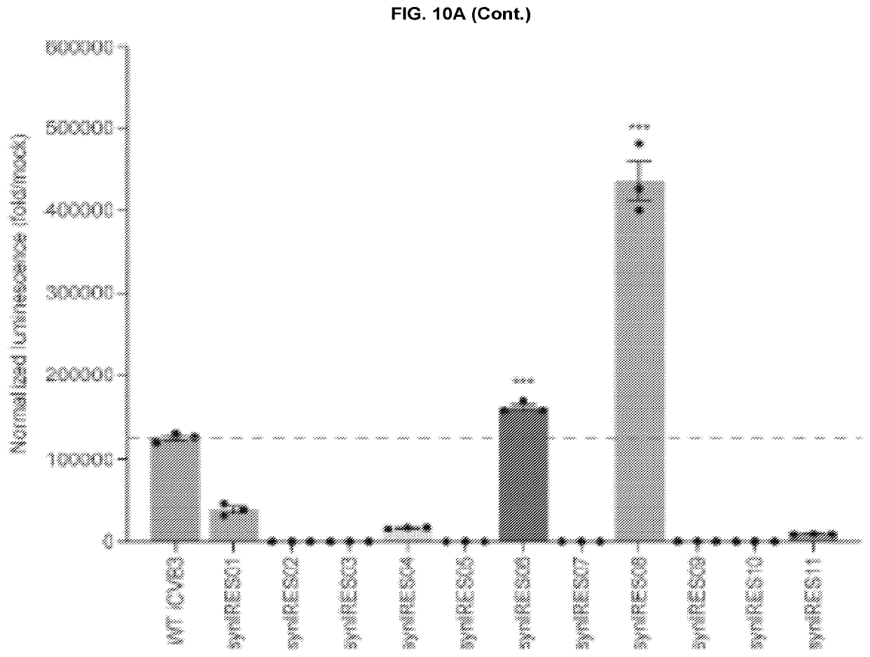

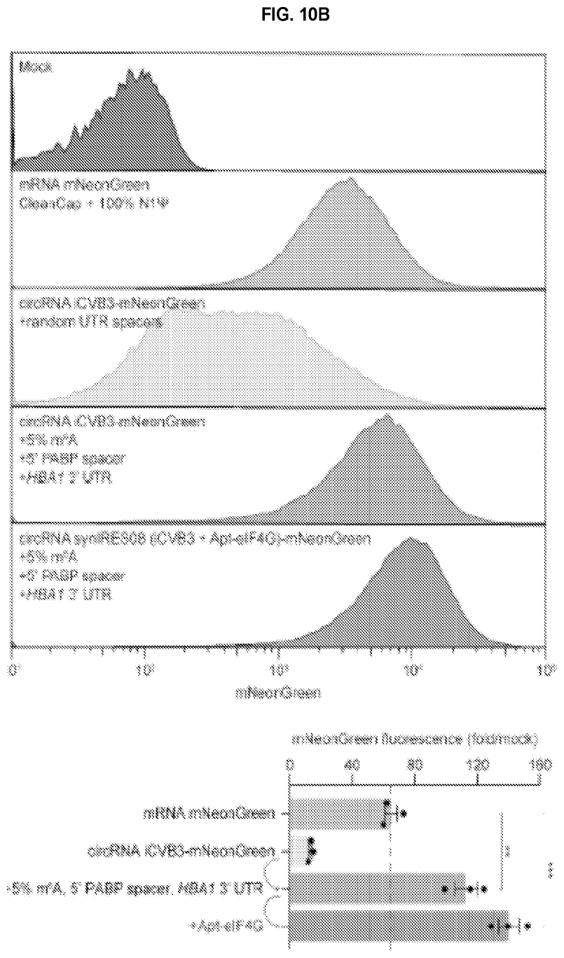

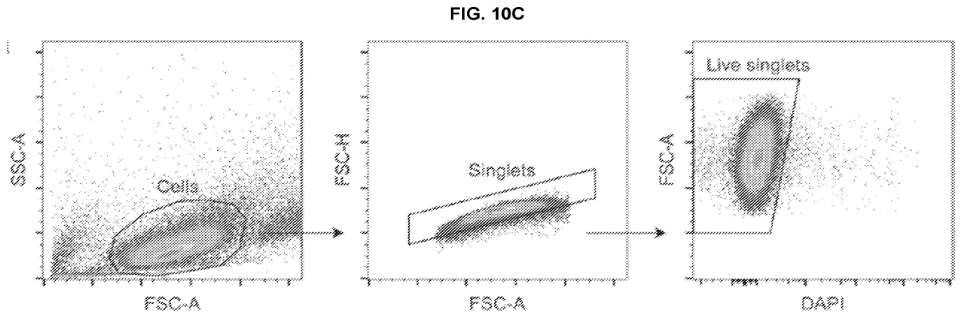

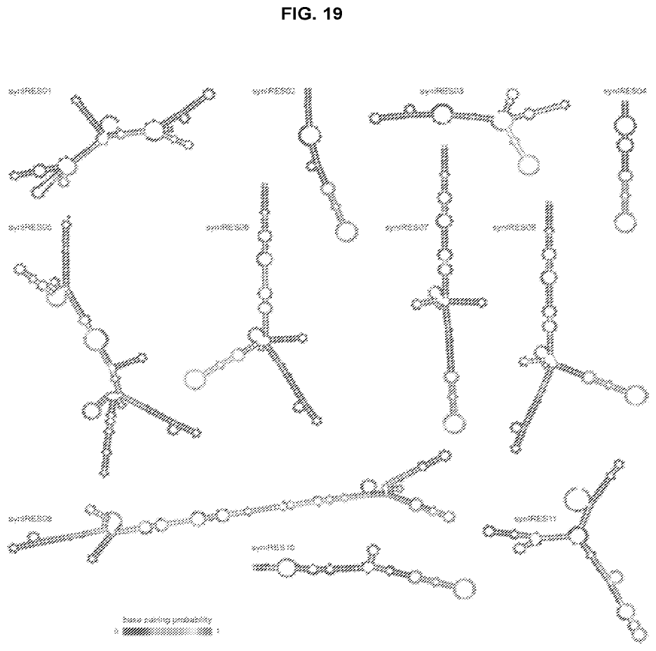

iCVB3 was engineered to have greater affinity for eIF4G. Apt-eIF4G, an eIF4G-recruiting aptamer, can improve cap-dependent translation when inserted in the 5′ UTR of mRNAs (Tusup 2018 Int. J. Med. Heal. Sci. (ISSN 2456-6063) 4, 29-37). Synthetic variants of the iCVB3 where Apt-eIF4G was inserted at hypothetically permissible regions within the IRES were generated (FIG. 10A). These positions were either within the flexible non-base-paired interdomain regions (synIRES01, 03, 05, 09, and 11), which were chosen to avoid aberrant Apt-eIF4G-linker interactions, or at the end of loop domains (synIRESO2, 04, 06, 07, 08, and 10), with removal of several wild-type nucleotides to smoothly transition from the stem-loop structure into Apt-eIF4G's RNA stem. In all cases, rational engineering choices were informed by in silico RNA structure prediction (FIG. 19). Using the NanoLuc assay, it was found that domain IV's cruciform structure was the most permissive to Apt-eIF4G insertion. Both synIRES06 and synIRES08, where Apt-eIF4G was inserted in the distal and proximal loops of domain IV, respectively, showed significantly improved translation over wild-type iCVB3. Conversely, insertion at the apical loop of domain IV completely abrogated translation, consistent with reports of an essential internal C-rich loop and GNRA tetraloops at this site (Garmarnik 2000 Nat. Methods 6, 343-345, Bhattacharyya 2006 Rna.3.2.2990 3, 60-68).

Using flow cytometry, the result was validated with a different reporter, mNeonGreen, a bright monomeric green fluorescent protein (Shaner 2013 Cell Res. 27, 315-328). Compared to CleanCap and 100% N1′-modified mRNA or unmodified circRNA with random 5′ and 3′ UTRs, 5% m6A-modified circRNA with the 5′ PABP spacer and HBA1 3′ UTR exhibited greater mNeonGreen expression (FIG. 10B). This was further improved by aptamer engineering of iCVB3 to include Apt-eIF4G. For gating strategy, see FIG. 10C.

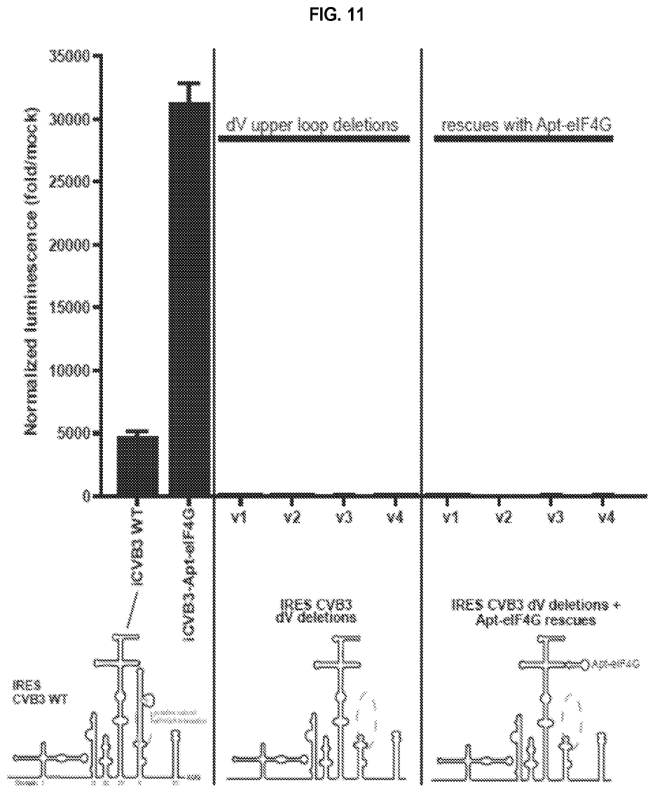

Experiments were conducted to determine if iCVB3 domain V eIF4G footprint deletions could be rescued through addition of Apt-eIF4G to the proximal loop of domain IV (FIG. 11). However, no recovery of translation was achieved by this strategy for any of the four variants. Prior toe-printing analysis deduced conformational changes in domain VI and the 3′ end of iCVB3 following the recruitment of eIF4G and eIF4A (de Breyne 2009; supra). The results indicate that these RNA conformational changes are important for proper ribosome assembly and that simply recruiting eIF4G locally is insufficient for translation initiation.

### Example 10: Identification of Robust Higher-Strength IRESs

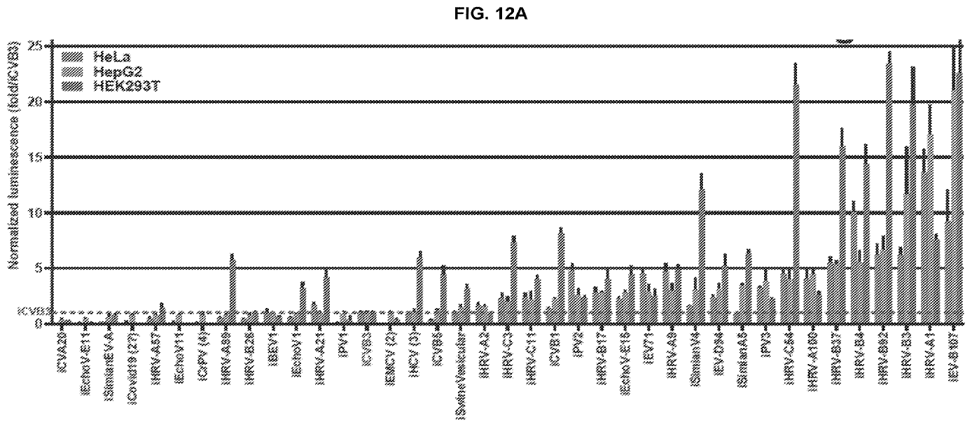

IRESs have evolved a variety of mechanisms to utilize host factors for initiating translation. Based on these mechanisms, IRESs have been categorized into several types—type 1 IRESs can be found in enteroviruses, type 2 in cardioviruses and aphthoviruses, type 3 in some picornaviruses, and type 4 in teschoviruses (Daijogo 2011). To further optimize circRNA expression, experiments were performed to identify IRESs with stronger translation than those previously described in the literature (Mokrejs 2006, Wesselhoeft 2018). Over several rounds of synthesis and testing, a number of IRESs spanning different types and species were characterized in circRNAs. IRESs representing canonical IRES types (type in parenthesis), such as from CVB3 (1), poliovirus 1 (PV1) (1), human rhinovirus Al (HRV-A1) (1), encephalomyocarditis virus (EMCV) (2), hepatitis C virus (HCV) (3), and cricket paralysis virus (CrPV) (4) were first investigated. Type 1 IRESs appeared to drive strong translation in the context of circRNAs (FIG. 12). These IRESs have extended structures that may allow them to scaffold a full set of ITAFs to initiate translation (Filbin 2009). The screen was expanded to include a large set of putative type 1 IRESs from the enterovirus genus, which were incorporated into circRNAs and assayed for NanoLuc translation.

In the screen, IRESs with stronger translation than iCVB3 across multiple cell lines were identified (FIG. 12). In particular, IRESs from the human rhinovirus B (HRV-B) and enterovirus B (EV-B) species drove strong circRNA translation.

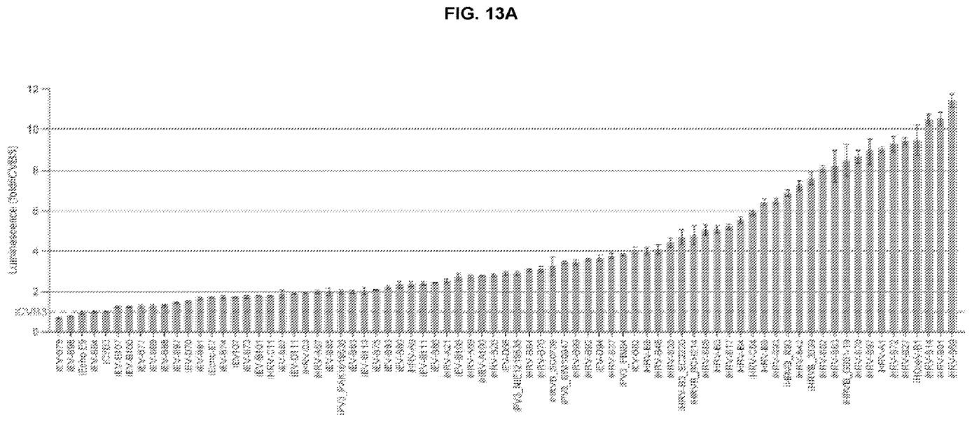

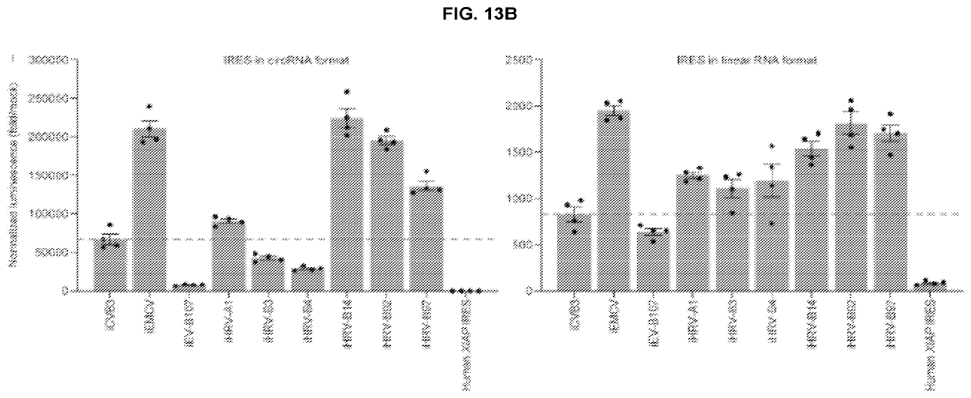

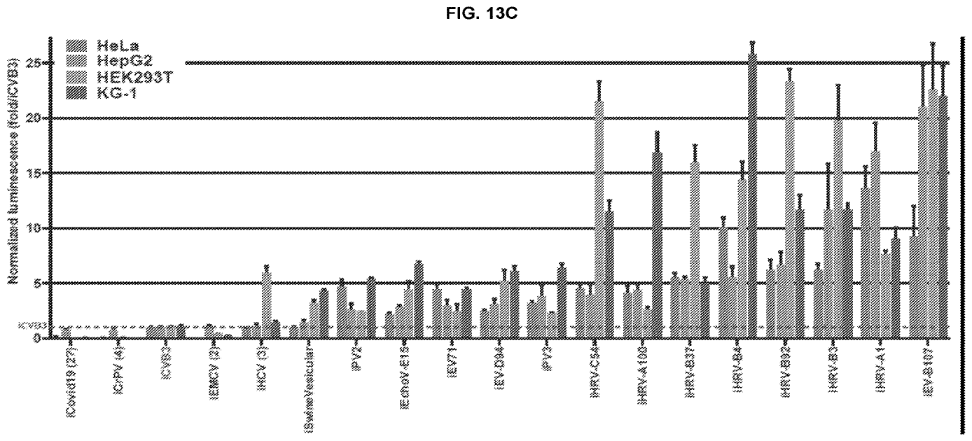

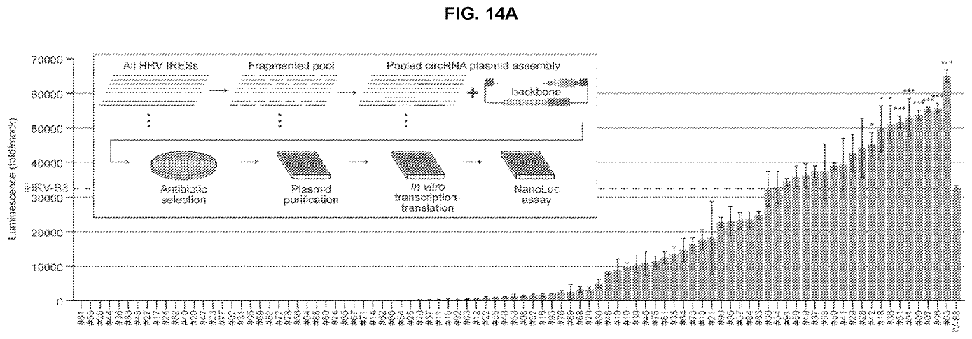

IRESs from every HRV-B and EV-B subspecies with a publicly available sequence on NCBI Virus were synthesized and incorporated into circRNA expression plasmids. An in vitro coupled transcription-translation (IVTT) approach, using circRNA expression plasmids rather than purified circRNAs as the input material, was used (FIG. 13A). In the IVTT-based NanoLuc assay, a large number of HRV-B and EV-B IRESs with greater translational activity than iCVB3 were found. Some of these IRESs were validated in cellulo using purified circRNAs (FIG. 13B). While many hits turned out to be false positives, the discovery of iHRV-B92 and iHRV-B97 as higher-strength IRESs were recapitulated. When these same IRESs were also tested in a linear RNA format, relative differences in translation strength held, but with a 100-fold reduction in absolute expression compared to circRNAs (FIG. 13B). For the strongest IRESs, NanoLuc translation was tested in four different cell lines and it was found that the many drove efficient translation independent of cell type (FIG. 13C). At the same time, some IRESs demonstrated stronger translation in a specific cell type, such as HEK293T cells for iHCV and iHRV-C54 and KG-I cells for iHRV-A100 and iHRV-B4.

### Example 11: Synthetic IRES Engineering Through Unbiased DNA Shuffling

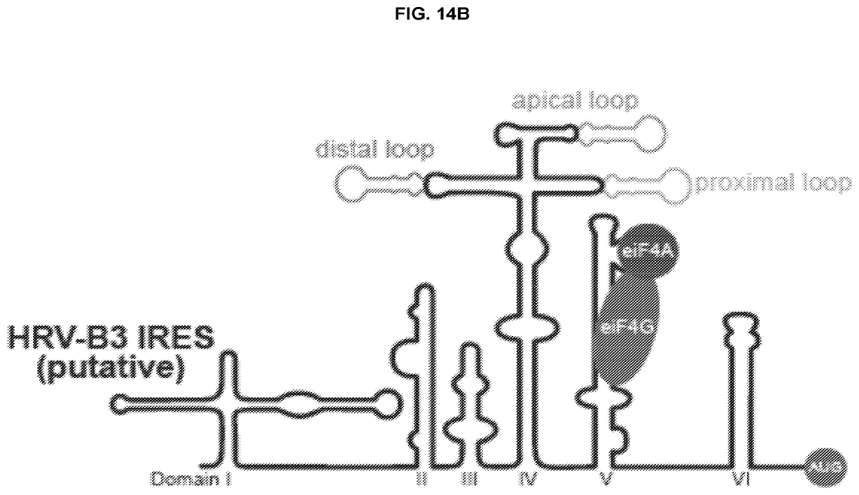

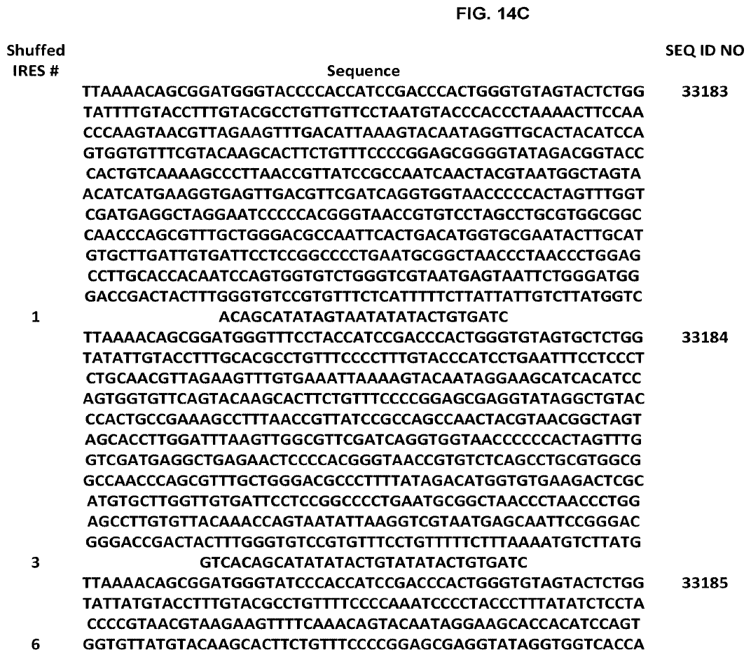

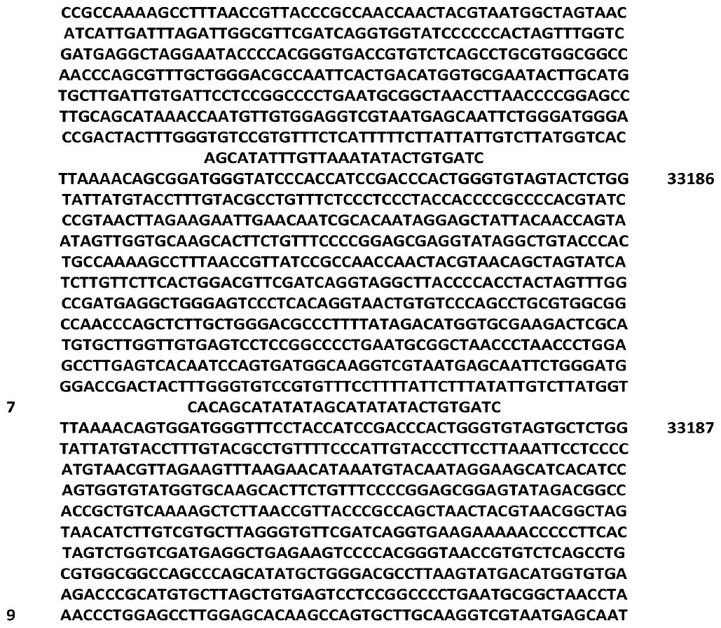

DNA shuffling is an unbiased approach commonly used to generate large diverse libraries for selecting novel engineered proteins (Michnick 1999 Nat. Biotechnol. 1999 1712 17, 1159-1160). Shuffling particularly makes sense over other library generating strategies, such as point mutagenesis, when a homologous family of related proteins is available to act as seed templates for the shuffling reaction. Because the strongest translation overall was observed with IRESs from HRV, DNA shuffling by fragmenting 41 HRV IRESs and cloning the resulting pool into circRNA plasmids (FIG. 14A). 93 circRNA expression plasmids with unique shuffled IRESs were isolated and their translation strength measured using an IVTT assay, with iHRV-B3 as an internal benchmarking control. From these 93 shuffled IRESs, nine with significantly stronger translational activity than wild-type iHRV-B3 were identified, illustrating the ability of IRES shuffling to engineer improved IRESs for circRNA applications (FIG. 14C).

### Example 12: Validation of Apt-eIF4G IRES Engineering with iHRV-B3

It was contemplated that the aptamer engineering approach with Apt-eIF4G might also improve translation for IRESs of indeterminate structure. To test this, the domain architecture of iHRV-B3 was predicted in silico (Gruber 2008 Nucleic Acids Res. 36, W70-W74), which identified six domains including a cruciform structure in domain IV (FIG. 14B). With a focus on loops within this cruciform structure, Apt-eIF4G insertions were performed at the distal, apical, and proximal loop locations, varying the length of the resulting stem by rationally inserting base-paired RNA nucleotides and validating the structure in silico. By assessing a range of stem lengths, a particular position for Apt-eIF4G most favorable to cooperative binding effects was identified. It was found that Apt-eIF4G insertions at the proximal loop of domain IV significantly improved circRNA translation compared to wild-type iHRV-B3, demonstrating the broader utility of the aptamer engineering strategy to synthesize stronger IRESs. As with iCVB3, apical loop insertions of Apt-eIF4G also destroyed iHRV-B3 activity, consistent with a predicted GNRA tetraloop in this region. When a double aptamer insertion of Apt-eIF4G was performed at both the distal and proximal loops, this greatly reduced circRNA translation.

### Example 13: The Effects of 2-Thiouridine (2ThioU) and 2′-O-Methylcitidine (2OMeC) Modifications on circRNA Translation

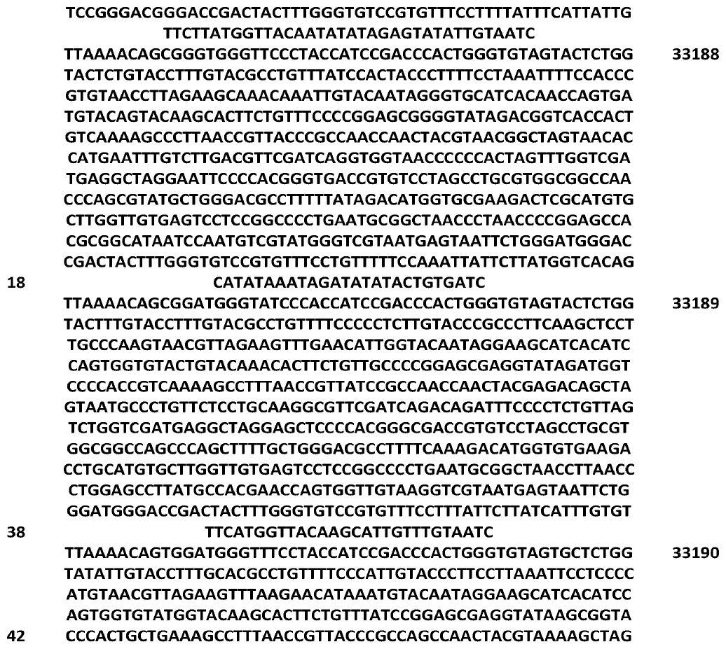

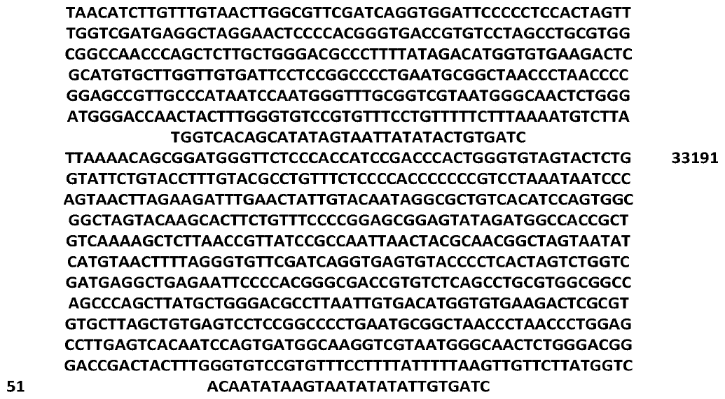

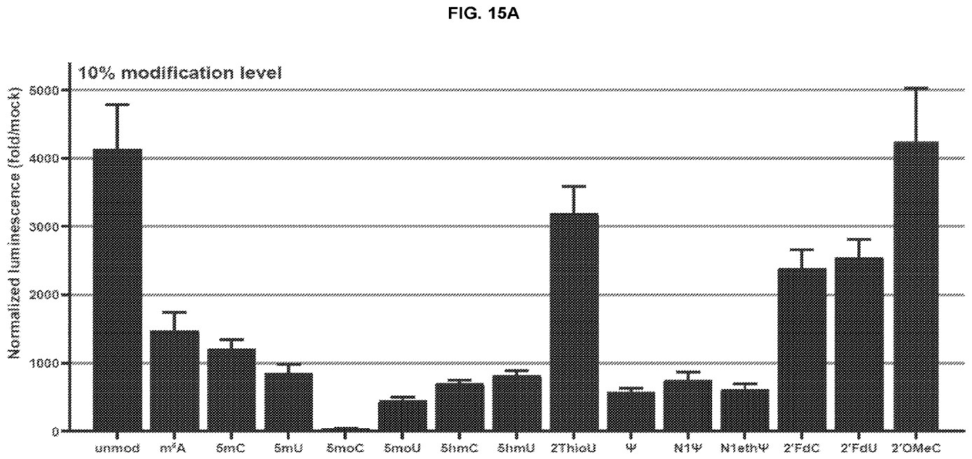

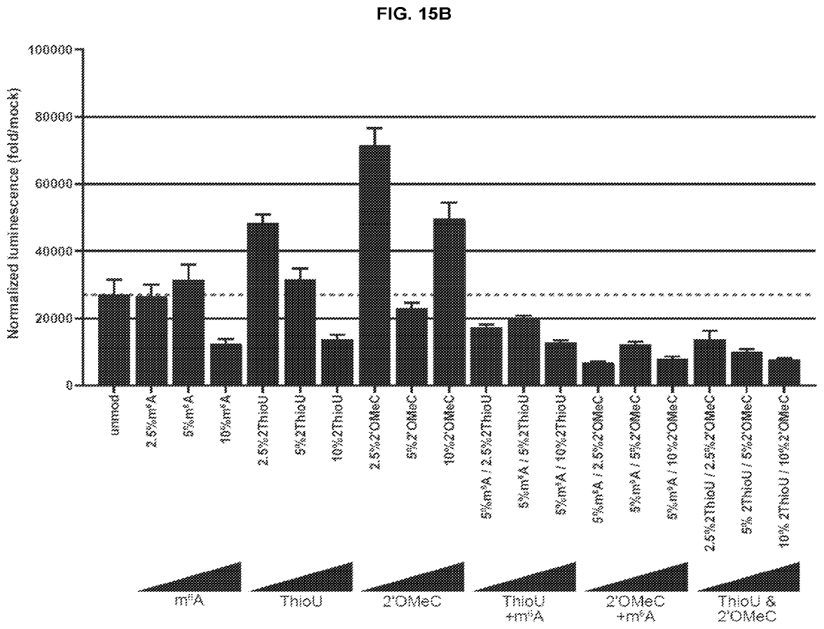

A panel of RNA modifications was analyzed, many with unknown prior effects on translation (FIG. 15A). In a first-pass synthesis, all the modifications were incorporated at a 10% level in circRNA synthesis. This incorporation level was chosen to allow for screening of modifications that lead to difficulty in T7 polymerase-based in vitro transcription or circRNA circularization, or severe blunting of translation. While most modifications had a deleterious effect on circRNA translation, 2-thiouridine (2ThioU) and 2′-O-methylcitidine (2OMeC) modifications improved circRNA translation. A further small-scale experiment exploring these modifications indicated that 2.5% incorporation level was the most advantageous for each modification (FIG. 15B). Dual incorporation of 2ThioU or 2OMeC or m⁶A in pairs blunted translation.

A final round of circRNA synthesis was performed comparing these newly-optimized circRNA modifications alongside modifications previously characterized for improving mRNA translation (FIG. 16A). Alongside translation strength, RNA stability was characterized using an in vitro titrated digestion assay in fetal bovine serum (FBS). mRNA or circRNA was diluted with FBS and digested for 30 minutes at 37° C.. In this period, RNases present in the FBS digest RNA to nucleotides, which eliminates ethidium bromide stain in the agarose gel. While both mRNA and unmodified circRNA rapidly degraded fully in just 1.0% FBS, the addition of 5% m⁶A improved stability to full degradation at 2%. Interestingly, 2.5% 2ThioU and 2.5% 2OMeC modifications conferred resistance to degradation and fully degraded at 3% FBS.

These results indicate that the mechanism behind 2ThioU and 2OMeC-based translation enhancement is likely due to improved stability to RNases that allow for improved integrated translational output over the RNA's life. 2-thiouridine and 2′-O-methylcytidine are moderate and potent enhancers, respectively, of circRNA translation activity. RNA modifications substantially improve stability to RNase degradation and thus translation half-life. The above findings suggest that the same modifications that have previously been characterized to improve mRNA translation (e.g., 5-hydroxymethyluridine, 5-methyoxyuridine, 5-methylcitidine, pseudouridine, and N1-methylpseudouridine) do not function in the same way for circRNAs. Thus, circRNA-specific screening of RNA modifications is necessary for identifying modifications that function in this context.

The results of this Example further support that the specific level of circRNA modification needs to be titrated to optimally enhance translation. Thus, circRNA modifications may be synthesized to drive differing functionalities, such as modification to specifically improve circRNA half-life, to improve amenability to lipid nanoparticle packaging and delivery, or to target specific cell types or cellular organelles.

### Example 14: RNA Modifications Improve Translation Strength and Stability

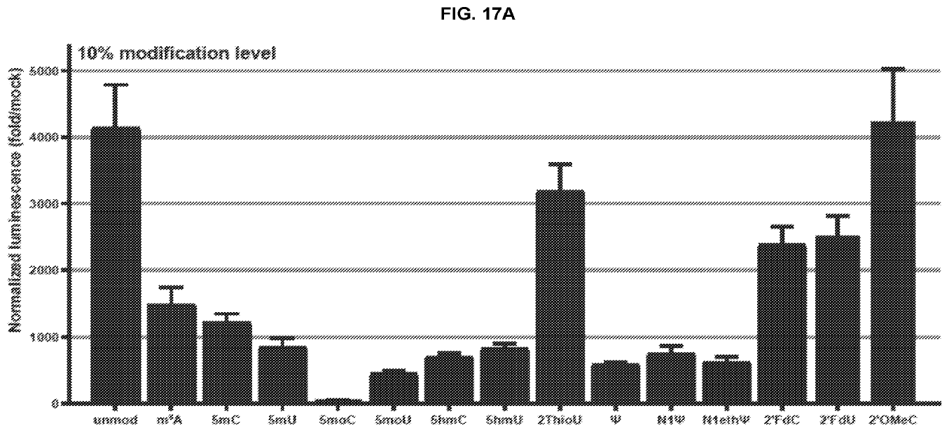

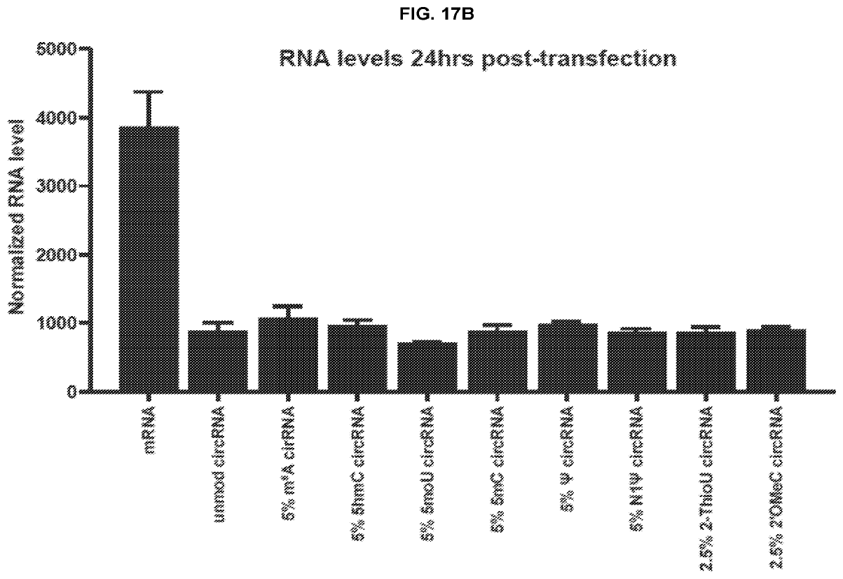

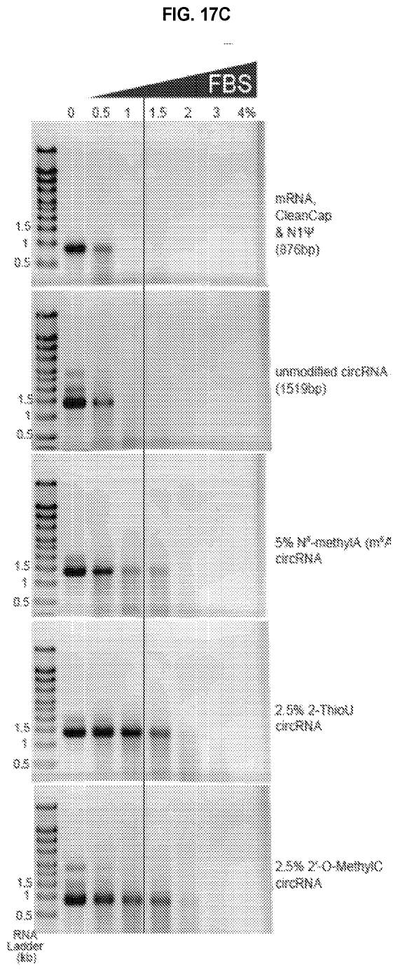

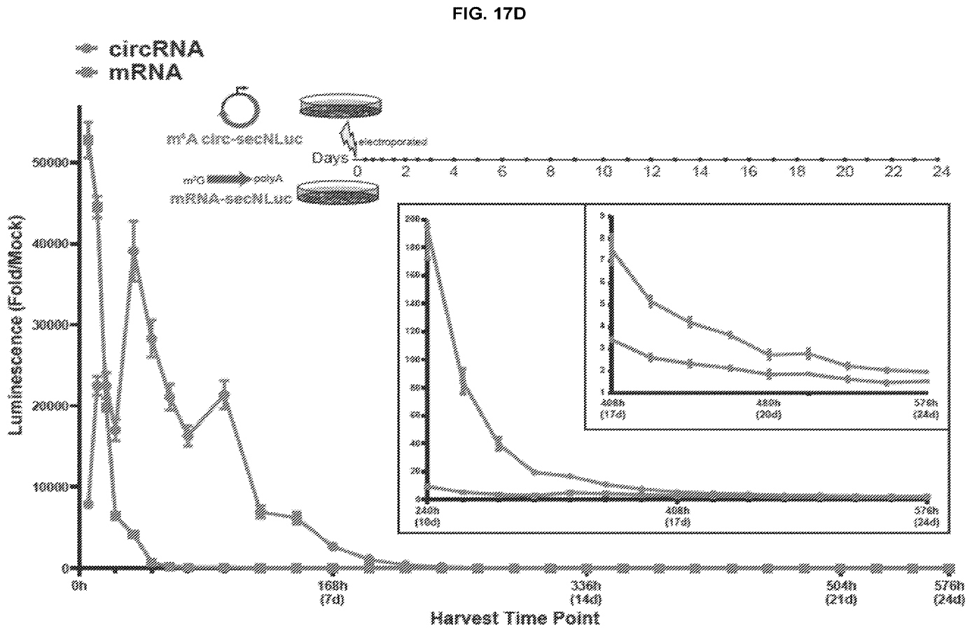

An unmodified circRNA encoding NanoLuc driven by the coxsackievirus B3 (CVB3) IRES (iCVB3) from the picornavirus family, with the translation cassette flanked by 50 bp random sequence spacers was used as a control. In separate syntheses, eight RNA modifications-5-methylcytidine (5mC), 5-methyluridine (5mU), 5-methoxycytidine (5moC), 5-methoxyuridine (5moU), 5-hydroxymethylcytidine (5hmC), 5-hydroxymethyluridine (5hmU), pseudouridine (ψ), and N1-methylpseudouridine (N1T)—that have demonstrated relevancy in improving mRNA translation (Karik6 2005); N6-methyladenosine (m6A) because of its relevance in modulating circRNA immunity (Chen 2019 Mol. Cell 67, 228-238.e5); and five RNA modifications—N1-ethylpseudouridine (NlethW), 2′-fluoro-2′-deoxycytidine (2′FdC), 2′-fluoro-2′-deoxyuridine (2′FdU), 2-thiouridine (2ThioU), and 2′-O-Methylcytidine (2′OMeC) -whose effects on RNA translation have not been described were incorporated into cirRNAs (FIG. 17A). On first-pass, all RNA modifications were tested at a 10% incorporation level to ensure a large effect size, and upon synthesis it was found that none of these modifications greatly reduced circRNA yield. When assayed for translation of NanoLuc, most modifications at 10% incorporation blunted translation compared to unmodified circRNA. However, 2ThioU and 2′OMeC inhibited translation to a lesser extent, indicating that further titration of their incorporation levels might improve translation strength.

Following further titration of RNA modifications at 2.5% and 5% incorporation optimized incorporation levels for eight RNA modifications in circRNAs were identified (FIG. 16A). Of these, 2′OMeC significantly improved translation while m6A and 2ThioU resulted in non-significant increases. Changes in translation were not due to differences in the amount of transfected RNA, which was equivalent among circRNA samples (FIG. 17B). Noticeably, nucleoside modifications known to improve mRNA translation such as N1T (Karik6 2005, Durbin 2016, Svitkin 2017) did not have the same effect in circRNAs.

A fetal bovine serum (FBS) degradation assay, which makes use of the endogenous RNases in FBS, was performed (FIG. 17C). CleanCap and 100% N1I-modified mRNA, the industry standard for mRNA-based therapies, was fully degraded by 1% FBS alongside unmodified circRNA. Conversely, circRNA containing 5% m6A was more resistant to nucleases and was not fully degraded until 2% FBS. These results indicate that nucleoside modification of circRNAs can confer stability against nucleases (FIG. 17C), which may help extend translation duration. However, when circRNAs are delivered into cells, certain RNA modifications improve translation strength despite having equivalent intracellular RNA stability (FIG. 16A).

Although circRNA translation in vitro was greatest with 2.5% 2′OMeC, attempts to combine this modification with m6A to block immune recognition abrogated translation efficiency. To compare the expression kinetics of 5% m6A-modified circRNA with CleanCap and 100% N1′-modified mRNA, a time course using secreted NanoLuc as the reporter was performed (FIG. 17D). mRNA and circRNA was electroporated into cells and media was harvested at time points out to 24 days, at which the NanoLuc signal was indistinguishable from background. While mRNA yielded a stronger maximum translation signal, translation rapidly dropped after 48 hours. On the other hand, circRNA translation peaked at 48 hours but continued yielding detectable expression out to almost 20 days.

### Example 15: Methods

circRNA synthesis

CircRNAs were synthesized using in vitro transcription (IVT) kits (HiScribe T7 High Yield RNA Synthesis Kit). IVT templates were PCR amplified (Q5 Hot Start High-Fidelity 2× Master Mix) for 30 cycles and column purified prior to RNA synthesis (DNA Clean & Concentrator-100. The following forward and reverse oligos were used circBB-T7promoter F:

(SEQ ID NO: 33181)
AAAAAAAAAAAAAAAAAAAAAAAAAAAggccagtgaattgtaatacgactcactataggg
circBB-intron-poly(A)
R:
(SEQ ID NO: 33182)
TTTTTTTTTTTTTTTTTTTTTTTTTTTTTTTTTTTTTTTTTTTTtagaaggcacagttaacgcggccgc

One microgram of circRNA template was used per 20 μL IVT reaction. Reactions were incubated overnight at 37° C. with shaking at 1,000 rpm with a heated lid. IVT templates were subsequently degraded with 2 μL of DnaseI per IVT reaction for 20 minutes at 37° C. with shaking at 1,000 rpm. The remaining RNA was column purified prior to further enzymatic reactions.

To isolate circRNAs, column purified RNA was digested with one unit of RnaseR per microgram of RNA for 60 minutes at 37° C. with shaking at 1,000 rpm. Samples were then column purified, quantified using a Nanodrop One spectrophotometer, and verified for complete digestion using an Agilent TapeStation. In some instances, due to reagent shortages, verification was performed with agarose gel under formamide-based denaturing conditions (NEB B0363S). In cases of incomplete digestion of linear RNAs, RnaseR digestion was repeated.

mRNA Synthesis

IVT templates for mRNA synthesis were PCR amplified (Q5 Hot Start High-Fidelity 2× Master Mix) for 30 cycles and column purified prior to RNA synthesis (DNA Clean & Concentrator-100). The reverse primer in this reaction incorporated a 100 bp poly(A) tail after the 3′ UTR. mRNA was then synthesized using IVT kits (HiScribe T7 High Yield RNA Synthesis Kit) with the following modifications: CleanCap AG (TriLink N-7113) was added to a 4 mM final concentration, and N1′ (TriLink N-1019) was fully substituted for UTP.

One microgram of mRNA template was used per 20 μL IVT reaction, Reactions were incubated for 2 hours at 37° C. with shaking at 1,000 rpm with a heated lid. IVT templates were subsequently degraded with 2 μL of DnaseI per IVT reaction for 20 minutes at 37° C. with shaking at 1,000 rpm. The remaining mRNA was column purified prior to use.

### RNA Gel Electrophoresis

1% agarose gels were prepared by melting RNase-free agarose in Tris-acetate-EDTA running buffer with addition of ethidium bromide. RNA was denatured in RNA loading buffer (Thermo Fisher) by diluting 1:1 volumetrically, heating to 72° C. for 3 minutes, and cooling on ice for 1 minute. RNA was loaded into each well and run at 100 V at room temperature until the bromophenol blue dye reached the edge of the gel. Images were taken using a Bio-Rad Gel Doc XR and Image Lab 5.2 software using the “SYBR-Safe” settings.

### Cell Culture and Transfection

HeLa (CCL-2), HEK293T (CRL-11268), HepG2 (HB-8065), and KG-1 (CCL-246) cells from ATCC were maintained with DMEM (Thermo Fisher) supplemented with 10% FBS (Gibco) and 1% penicillin-streptomycin (Gibco). For routine subculture, 0.25% TrypLE (Thermo Fisher) was used for cell dissociation. For the selection of transduced cells, puromycin (Thermo Fisher) was used at a final concentration of 1 μg/mL.

RNA delivery was achieved with TransIT-mRNA transfection, Lipofectamine transfection, or NEON electroporation. Within each experiment, the molar amount of mRNA or circRNA delivered and transfection method used was the same for all samples. For TransIT-mRNA transfections, 3 μL of TransIT-mRNA reagent (Mirus Bio) was used per microgram of circRNA. Besides this change, transfections were performed following manufacturer's instructions.

### In Vitro NanoLuciferase Assay

Cells were electroporated with the pGL4.54[luc2/TK]vector (Promega) expressing firefly luciferase and transfected with mRNA or circRNA 48 hours later. Cells were harvested at 24 hours post-transfection in 100 μL of passive lysis buffer (Promega) and lysed by rocking and pipetting for roughly 15 minutes at room temperature. Lysate was centrifuged at 4,000 rcf for 10 minutes to clear debris, and 5 μL of clarified lysate was transferred into a 384-well white-bottom assay plate (Perkin Elmer). To each well, 10 μL of ONE-Glo EX from the Promega Nano-Glo Dual-Luciferase Reporter Assay System was added, after which the plate was vortexed for 1 minute, incubated at room temperature for an additional 2 minutes, and read on a TECAN Infinite Pro microplate reader.

Samples were first measured for firefly luminescence, which was used as a constitutive control. To each well, 10 μL of freshly-made NanoDLR Stop & Glo Reagent was then added, after which the plate vortexed for 1 minute and incubated at room temperature for an additional 9 minutes before NanoLuc luminescence was read. Normalized luminescence per well was calculated by dividing NanoLuc signal by firefly luminescence. Within each experiment, normalized luminescence was displayed in terms of fold change relative to mock (no RNA) transfections.

mNeonGreen Flow Cytometry Assay

CircRNAs and mRNAs expressing mNeonGreen driven by different iterations of RNA backbones were electroporated into HeLa cells via NEON electroporation. At 24 hours post-electroporation, cells were lifted using warmed TrypLE (Thermo Fisher), which was quenched with DMEM (Thermo Fisher), and incubated in PBS containing propidium iodide live-dead stain (Thermo Fisher) at room temperature for 15 minutes. Cells were analyzed via flow cytometry on an Attune N×T with the same voltages applied to all conditions. At least 50,000 live singlet cells were recorded per sample.

### In Vitro Transcription-Translation

Coupled IVTT was performed using the 1-Step Human Coupled IVT kit (Thermo Scientific) following manufacturer's instructions. Briefly, circRNA plasmids were incubated with HeLa lysate, accessory proteins, and the reaction mix for at least 90 minutes. An aliquot from each reaction was then used to measure NanoLuc activity as described above.

### Western Blotting

HeLa cells were lysed 24 hours after electroporation using RIPA Lysis and Extraction Buffer (Thermo Fisher) containing Halt Protease and Phosphatase Inhibitor Cocktail (Thermo Fisher). The resulting lysate was clarified by centrifugation and quantified for protein using bicinchoninic acid. Subsequently, 10 g of total protein from each sample was separated on a Bis-Tris gel and transferred to a nitrocellulose membrane using the iBlot 2 Gel Transfer Device. After blocking with 5% bovine serum albumin in 0.1% Tween-20 diluted in PBS for one hour at room temperature, the membrane was stained with a 1:500 dilution of anti-NanoLuc antibody (R&D Systems, MAB10026) in blocking buffer overnight at 4° C. Following washes, the membrane was then incubated with a 1:10,000 dilution of IRDye 680RD goat anti-mouse secondary antibody (LI-COR Biosciences, 926-68070) and visualized on an Odyssey CLx Imaging System (LI-COR Biosciences).

### RNA Structure Predictions

RNA structures were predicted using the RNAfold web server (rna.tbi.univie.ac.at/cgi-bin/RNAWebSuite/RNAfold.cgi) with default settings except for deselecting “avoid isolated base pairs.” The optimal secondary structure based on minimal free energy prediction was subsequently used to represent the RNA sequence.

### Embodiments. Exemplary embodiments of the disclosure are shown below

1. A circular RNA molecule comprising an internal ribosome entry site (IRES) sequence operably linked to a protein-coding sequence; wherein the IRES sequence is a viral sequence; and wherein the protein-coding sequence encodes a non-viral protein.
2. The circular RNA molecule of claim 1, wherein the non-viral protein is a mammalian protein.
3. The circular RNA molecule of claim 1, wherein the non-viral protein is a human protein.
4. The circular RNA molecule of claim 1, wherein the IRES is a Type 1 IRES.
5. The circular RNA molecule of claim 1, wherein the IRES is an enterovirus IRES.
6. The circular RNA molecule of claim 1, wherein the IRES is a human rhinovirus (HRV) IRES.
7. The circular RNA molecule of any one of claims 1-4, wherein the IRES is any one of the IRES listed in Table 7.
8. The circular RNA molecule of any one of claims 1-4, wherein the IRES is any one of the following IRES: iEMCV, iHCV, iCVB5, iSwineVesicular, iHRV-A2, iHRV-C3, iHRV-C11, iCVB1, iPV2, iHRV-B17, iEchoV-E15, iEV71, iHRV-A9, iSiminanV4, iEV-D94, iSimianA5, iPV3, iHRV-C54, iHRV-A100, iHRV-B37, iHRV-B4, iHRV-B92, iHRV-B3, iHRV-A1, iEV107, or a fragment or derivative thereof.
9. The circular RNA molecule of any one of claims 1-4, wherein the IRES is any one of the following IRES: iEV-B83, iHRV-A57, iHRV-B35, iHRV-B4, iEV-D68, iHRVB_R93, iHRV-B5, iHRVB-B52, iHRVB-B93, iHRV-B84, iHRV-B83_SC2220, iHRV-B72, iHRV-B69, iHRVB SC0739, iHRV-B91, iHRV-B42, iHRV-B6, iHRV-B83, iHRV-B48, iHRV-B99, iHRV-B79, iHRV-B97, iHRV-B27, iHRVB 3039, iHRVB-B14, iCosV-B1, or a fragment or derivative thereof.
10. The circular RNA molecule of any one of claims 1-4, wherein the IRES is iCVB3, or a fragment or derivative thereof.
11. The circular RNA molecule of any one of claims 1-4, wherein the IRES is iHRV-B3, or a fragment or derivative thereof.
12. A circular RNA molecule comprising a synthetic internal ribosome entry site (IRES) sequence operably linked to a protein-coding sequence.
13. The circular RNA molecule of claim 12, wherein the synthetic IRES sequence is upstream of said protein coding sequence.
14. The circular RNA molecule of claim 12, wherein the synthetic IRES sequence comprises at least one aptamer.
15. The circular RNA molecule of any one of claims 12-14, wherein the aptamer is a wildtype aptamer.
16. The circular RNA molecule of claims 12-14, wherein the aptamer is a mutant aptamer.
17. The circular RNA molecule of claim 16, wherein the aptamer is modified to have an extended stem region.
18. The circular RNA molecule of any one of claims 13-17, wherein the aptamer is positioned within the secondary structure of the IRES so that is spatially proximal to portion of the IRES responsible for translation initiation.
19. The circular RNA molecule of any one of claims 13-18, wherein the aptamer does not interrupt the native eIF4G binding site of the IRES and does not interrupt a native GRNA tetraloop within the IRES.
20. The circular RNA molecule of any one of claims 13-19, wherein the aptamer is an eIF4G-binding aptamer.
21. The circular RNA molecule of claim 20, wherein the eIF4G-binding aptamer is encoded by the sequence of SEQ ID NO: 99.
22. The circular RNA of any one of claims 12-21, wherein the IRES is a Type 1 IRES.
23. The circular RNA of any one of claims 12-22, wherein the IRES is a modified enterovirus IRES.
24. The circular RNA of any one of claims 12-22, wherein the IRES is a modified human rhinovirus (HRV) IRES.
25. The circular RNA molecule of claim 13, wherein the synthetic IRES sequence is a modified iCVB3 IRES.
26. The circular RNA molecule of claim 25, wherein the modified iCVB3 IRES comprises an aptamer inserted in domain I, II, III, IV, V, VI or VII thereof.
27. The circular RNA molecule of claim 25, wherein the modified iCVB3 IRES comprises an aptamer inserted in domain IV thereof.
28. The circular RNA molecule of any one of claims 25-27, wherein the aptamer is modified to have an extended stem region.
29. The circular RNA molecule of any one of claims 25-27, wherein the aptamer is positioned within the secondary structure of the IRES so that is spatially proximal to portion of the IRES responsible for translation initiation.
30. The circular RNA molecule of any one of claims 25-27, wherein the aptamer does not interrupt the native eIF4G binding site of the IRES and does not interrupt a native GRNA tetraloop within the IRES.
31. The circular RNA molecule of claim 13, wherein the synthetic IRES sequence is a modified iHRV-B3 IRES.
32. The circular RNA molecule of claim 31, wherein the modified iHRV-B3 IRES comprises an aptamer inserted in domain I, II, III, IV, V, or VI thereof.
33. The circular RNA molecule of claim 31, wherein the modified iHRV-B3 IRES comprises an aptamer inserted in domain IV thereof.
34. The circular RNA molecule of any one of claims 32-33, wherein the aptamer is modified to have an extended stem region.
35. The circular RNA molecule of any one of claims 32-34, wherein the aptamer is positioned within the secondary structure of the IRES so that is spatially proximal to portion of the IRES responsible for translation initiation.
36. The circular RNA molecule of any one of claims 35-35, wherein the aptamer does not interrupt the native eIF4G binding site of the IRES and does not interrupt a native GRNA tetraloop within the IRES.
37. The circular RNA molecule of any of the preceding claims, wherein said circular RNA molecule comprises at least one 2-thiouridine (2ThioU) or at least one 2′-O-methylcitidine (20MeC).
38. The circular RNA molecule of claim 37, which comprises at least one 2-thiouridine.
39. The circular RNA molecule of claim 38, which comprises about 2% to about 5% 2-thiouridine.
40. The circular RNA molecule of claim 39, which comprises about 2.5% 2-thiouridine.
41. The circular RNA molecule of claim 37, which comprises at least one 2′-O-methylcitidine.
42. The circular RNA molecule of claim 41, which comprises about 2% to about 5% 2′-O-methylcitidine.
43. The circular RNA molecule of claim 42, which comprises about 2.5% 2′-O-methylcitidine.
44. The circular RNA molecule of any of the preceding claims, wherein said molecule comprises a nucleic acid spacer upstream of said IRES.
45. A nucleic acid that encodes the circular RNA molecule of any one of claims 1-44.
46. A composition comprising the circular RNA molecule of any one of claims 1-44 or the nucleic acid of claim 45
47. A host cell comprising the circular RNA molecule of any one of claims 1-44 or the nucleic acid of claim 45.
48. A method of producing a protein in a cell, the method comprising contacting a cell with the circular RNA molecule of any one of claims 1-44 or the nucleic acid of claim 45 under conditions whereby the protein-coding nucleic acid sequence of the circular RNA is translated and the protein is produced in the cell.
49. A method of producing a protein in vitro, the method comprising contacting a cell-free extract with the circular RNA molecule of any one of claims 1-44 or the nucleic acid of claim 45 under conditions whereby the protein-coding nucleic acid sequence of the circular RNA is translated and the protein is produced.
50. A protein produced by the method of any one of claims 48-49.

SEQUENCE APPENDIX

| | | SEQ |
|---|---|---|
| | | ID |
| Description | Sequence | NO: |
| iPV1 IRES | ttaaaacagctctggggttgtacccaccccagaggcccacgtggcggctagtactccggtattg | 1 |
| | cggtacccttgtacgcctgttttatactcccttcccgtaacttagacgcacaaaaccaagttcaata | |
| | gaagggggtacaaaccagtaccaccacgaacaagcacttctgtttccccggtgatgtcgtatag | |
| | actgcttgcgtggttgaaagcgacggatccgttatccgcttatgtacttcgagaagcccagtacc | |
| | acctcggaatcttcgatgcgttgcgctcagcactcaaccccagagtgtagcttaggctgatgagt | |
| | ctggacatccctcaccggtgacggtggtccaggctgcgttggcggcctacctatggctaacgc | |
| | catgggacgctagttgtgaacaaggtgtgaagagcctattgagctacataagaatcctccggcc | |
| | cctgaatgcggctaatcccaacctcggagcaggtggtcacaaaccagtgattggcctgtcgtaa | |
| | cgcgcaagtccgtggcggaaccgactactttgggtgtccgtgtttccttttattttattgtggctgct | |
| | tatggtgacaatcacagattgttatcataaagcgaattggattggccatccggtgaaagtgagac | |
| | tcattatctatctgtttgctggatccgctccattgagtgtgtttactctaagtacaatttcaacagttatt | |
| | tcaatcagacaattgtatcata | |
| iPV2 IRES | ttaaaacagctctggggttgttcccaccccagaggcccacgtggcggccagtacactggtatc | 2 |
| | gcggtacctttgtacgcctgttttatactcccttcccccgtaacttagaagcacaacgtccaagttc | |
| | aatagaagggggtacaaaccagtaccaccacgaacaagcacttctgttcccccggtgaggctg | |
| | tataggctgtttccacggctaaaagcggctgatccgttatccgctcatgtacttcgagaagcctag | |
| | tatcaccttggaatcttcgatgcgttgcgctcaacactcaaccccagagtgtagcttaggtcgatg | |
| | agtctggacgttcctcaccggcgacggtggtccaggctgcgttggcggcctacctgtggccca | |
| | aagccacaggacgctagttgtgaacaaggtgtgaagagcctattgagctacctgagagtcctc | |
| | cggcccctgaatgcggctaatcctaaccacggagcaggcagtggcaatccagcgaccagcct | |
| | gtcgtaacgcgcaagttcgtggcggaaccgactactttgggtgtccgtgtttccttttatttttacaa | |
| | tggctgcttatggtgacaattattgatagttatcataaagcaaattggattggccatccggtgagaa | |
| | tttgattattaaattactctcttgttgggattgctcctttgaaatcctgtgcactcacacctattggaatt | |
| | acctcattgttgagatattattaccact | |
| iPV3 IRES | ttaaaacagctctggggttgctcccaccccagaggcccacgtggcggccagtactctggtattg | 3 |
| | cggtacctttgtacgcctgttttatactccctccccccgtgcaactttagaagaaatccacaaagtt | |
| | caatagagggggtgcaaaccagcaccaccacgaacaagcacttctgtttccccggtgatgtcg | |
| | tataagctgtacccacggctgaaggcgacggatccgttatccgcttgagtacttcgagaagcct | |
| | agtattaccttggaatcttcgacgcgttgcgctcagcactctaccccgagtgtagcttaggtcgat | |
| | gagtctgggcacgccccaccggcgacggtggcccaggctgcgttggcggcctacccatggc | |
| | tatcaccatgggacgctagttgtgaacaaggtgtgaagagcctattgagctacccaagagtcct | |
| | ccggcccctgaatgcggctaatcctaaccacggagcaagtgtcctcaacccaggggatggctt | |
| | gtcgtaacgcgaaagtctgtggcggaaccgactactttgggtgtccgtgtttccttttatttttatgt | |
| | atggctgcttatggtgacaatcaaaggttgttaccataaagcaatttggattggccatccggtgag | |
| | aatcaaacatattatctacctgtttgttgggttttcttcttttacttgaacaatacctctaataataactg | |
| | ctatattgtcaataagacattatcatcaca | |
| iHRV1 IRES | ttaaaactgggtgtgggttgttcccacccacaccacccaatgggtgttgtactctgttattccggta | 4 |
| | actttgtacgccagtttttccctcccctccccatccttttacgtaacttagaagttttaaatacaagac | |
| | caatagtaggcaactctccaggttgtctaaggtcaagcacttctgtttccccggttgatgttgatat | |
| | gctccaacagggcaaaaacaacagataccgttatccgcaaagtgcctacacagagcttagtag | |
| | gattctgaaagatctttggttggtcgttcagctgcatacccagcagtagaccttgcagatgaggct | |
| | ggacattccccactggtaacagtggtccagcctgcgtggctgcctgcgcacctctcatgaggtg | |
| | tgaagccaaagatcggacagggtgtgaagagccgcgtgtgctcactttgagtcctccggcccc | |
| | tgaatgcggctaaccttaaacctgcagccatggctcataagccaatgagtttatggtcgtaacga | |
| | gtaattgcgggatgggaccgactactttgggtgtccgtgtttcactttttcctttattaattgcttatg | |
| | gtgacaatatatatattgatatatattggcatc | |
| iHRV2 IRES | ttaaaactggatccaggttgttcccacctggatttcccacagggagtggtactctgttattacggta | 5 |
| | actttgtacgccagttttatctcccttcccccatgtaacttagaagtttttcacaaagaccaatagcc | |
| | ggtaatcagccagattactgaaggtcaagcacttctgtttccccggtcaatgttgatatgctccaa | |
| | cagggcaaaaacaactgcgatcgttaaccgcaaagcgcctacgcaaagcttagtagcatctttg | |
| | aaatcgtttggctggtcgatccgccatttcccctggtagacctggcagatgaggctagaaatacc | |
| | ccactggcgacagtgttctagcctgcgtggctgcctgcacaccctatgggtgtgaagccaaac | |
| | aatggacaaggtgtgaagagccccgtgtgctcgctttgagtcctccggcccctgaatgtggcta | |
| | accttaaccctgcagctagagcacgtaacccaatgtgtatctagtcgtaatgagcaattgcggga | |
| | tgggaccaactactttgggtgtccgtgtttcactttttcctttatatttgcttatggtgacaatatatac | |
| | aatatatatattggcacc | |
| iHRV-B3 IRES | ttaaaacagcggatgggtaccccaccatccgacccactgggtgtagtactctggtacttcgtacc | 6 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaattccccacgggtgaccgtgtcctagcctgcgtggcggccaacccagcttatg | |
| | ctgggacgcctttttatagacatggtgtgaagactcgcatgtgcttggttgtgattcctccggccc | |
| | ctgaatgcggctaaccttaaccctggagccttgtgtcacaaaccagtgatgataaggtcgtaatg | |
| | agcaattccgggacgggaccgactactttgggtgtccgtgtttcttatttttcttattattgtcttatgg | |
| | tcacagcatatatataacatatactgtgatc | |
| iCVA1 IRES | ttaaaacagctctggggttgttcccaccccagaggcccacgtggcggccagtactctggtattg | 7 |
| | cggtacctttgtacgcctgttttatatcccctccccaagtaactttagaagcaattcacaaagttcaa | |
| | tagagggggtacaaaccagtaccaccatgaacaagcacttctgtttccccggtgaagctgtata | |
| | gactgttcccacggtcgaaaatggcagatccgttatccgctcacgtacttcgagaagcctagtat | |
| | catcttggaatcttcgacgcgttgcgctcagcactctaccccgagtgtagcttaggccgatgagt | |
| | ctgggcataccccaccggcgacggtggcccaggctgcgttggcggcctacccatggctaatg | |
| | tcatgggacgctagttgtgaacaaggtgtgaagagcctattgagctacttgagagtcctccggc | |
| | ccctgaatgcggctaatcctaaccatggagcaagtgcccacaagccagtgggtggcttgtcgt | |
| | aatgcgcaagtctatggcggaaccgactactttgggtgtccgtgtttccttttattcttattatggct | |
| | gcttatggtgacaacctaagattgttgtcatatagcttttggattggccatccggtgatctttgagat | |
| | taatataccttccttattacttcatagtactcatttattcattacactactgtagttaca | |
| iCVA16 IRES | ttaaaacagcctgtgggttgttcccacccacagggcccactgggcgctagcacactgattctac | 8 |
| | gggatccttgtgcgcctgttttataatccttcccccaagtagcaacttagaagctttgtacaatcac | |
| | gaccagtagtgggcgtggcgcgccagtcacgtcttggtcaagcacttctgtatccccggactga | |
| | gtatcaatagactgctcacgcggttgaaggagaaaacgttcgttacccggctaactacttcgag | |
| | aaacccagtagcaccgtgaaagttgcggagtgtttcgctcagcacttcccccgtgtagatcagg | |
| | tcgatgagtcactgtaaaccccacgggcgaccgtgacagtggctgcgttggcggcctgcccat | |
| | ggggtaacccatgggacgctctaatacagacacggtgtgaagagtctattgagctagttagtag | |
| | tcctccggcccctgaatgcggctaatcctaactgcggagcgcgcaccctcaacccagggggc | |
| | ggcgtgtcgtaatgggtaactctgcagcggaaccgactactttgggtgtccgtgtttccttttattc | |
| | cttattggctgcttatggtgacaattgaaaagttgttaccatatagctattggattggccatccggtg | |
| | tctaatagagctattgtttacttatttattggatacgtccctcttaatctcaaagtcattcaaactcttga | |
| | ttatatattgctccttaactgtaagaa | |
| iCVB2 IRES | ttaaaacagcctgtgggttgatcccacccacagggcccactgggcgctagcacactggtattac | 9 |
| | ggtacctttgtgcgcctgttttatctacccttgcccaatgcaacatagaagcttagtccaaaaggtc | |
| | aataggcagcccagtgcaccaactgggtccagaccaagcacttctgttaccccggaccaagta | |
| | tcagtaggctgctcacgcggtgaaggagaaaacgttcgttacccggccaactacttcgagaaa | |
| | cctagtagcaccatgaaggttgcgcagtgtttcgctccgcacagccccagtgtagatcaggtcg | |
| | atgagccaccgcattccccacggggtgaccgtggcgggtggctgcgttggcggcctgcccat | |
| | ggggtaacccatgggacgcttcaatactgacatggtgtgaagagtctattgagctagttggtagt | |
| | cctccggcccctgaatgcggctaatcctaactgcggagcgtgcgcttgcaacccagtgagtag | |
| | cacgtcgtaatgggtaactctgcagcggaaccgactactttgggtgtccgtgtttcctttattcctt | |
| | gtctggctgcttatggtgacaattgagagattgttgccatatagctattggattggccatccggtat | |
| | ctaacagggcgatcatttacctgtttgttggtttcatacccttgaatcttgaggtttataataccctca | |
| | ctcttataataaaactcaatacatcaag | |
| iCVB3 IRES | ttaaaacagcctgtgggttgatcccacccacagggcccattgggcgctagcactctggtatcac | 10 |
| | ggtacctttgtgcgcctgttttataccccctcccccaactgtaacttagaagtaacacacaccgat | |
| | caacagtcagcgtggcacaccagccacgttttgatcaagcacttctgttaccccggactgagtat | |
| | caatagactgctcacgcggttgaaggagaaagcgttcgttatccggccaactacttcgaaaaac | |
| | ctagtaacaccgtggaagttgcagagtgtttcgctcagcactaccccagtgtagatcaggtcgat | |
| | gagtcaccgcattccccacgggcgaccgtggcggtggctgcgttggcggcctgcccatggg | |
| | gaaacccatgggacgctctaatacagacatggtgcgaagagtctattgagctagttggtagtcct | |
| | ccggcccctgaatgcggctaatcctaactgcggagcacacaccctcaagccagagggcagtg | |
| | tgtcgtaacgggcaactctgcagcggaaccgactactttgggtgtccgtgtttcattttattcctat | |
| | actggctgcttatggtgacaattgagagattgttaccatatagctattggattggccatccggtga | |
| | ccaatagagctattatatatctctttgttgggtttataccacttagcttgaaagaggttaaaacattac | |
| | aattcattgttaagttgaatacagcaaa | |
| IEV-A71 IRES | ttaaaacagcctgtgggttgcacccactcacagggcccacgtggcgctagcactctggttctac | 11 |
| | ggaacctttgtgcgcctgttttacgccccttcccccaatttgcaacttagaagcaatacacaacac | |
| | tggtcaacagtaggcatggcgcaccagccatgtcttgatcaagcacttctgtttccccggaccg | |
| | agtatcaatagactgctcacgcggttgaaggagaaagcgtccgttatccggctaactacttcga | |
| | gaaacttagtaacaccattgaagctgcagagtgtttcgctcggcacttcccccgtgtagatcagg | |
| | tcgatgagtcactgcaatccccacgggtgaccgtggcagtggctgcgctggcggcctgcctat | |
| | ggggtaacccataggacgctctaatgcggacatggtgcgaagagtctattgagctagttagtgg | |
| | tcctccggcccctgaatgcggctaatcctaactgcggagcacatgccttcaatccagagggtag | |
| | tgtgtcgtaatgggcaactctgcagcggaaccgactactttgggtgtccgtgtttccttttattctta | |
| | tattggctgcttatggtgacaattacagaattgttaccatatagctattggattggccatccggtgtg | |
| | caatagagctattatatacctatttgttggctttgtgccactaaccttaaaatctataaccaccctcg | |
| | actttatactaaccctcaatacagtcaaac | |
| IEV-A120 IRES | ttaaaacagcctgtgggttgtacccacccacagggcccactgggcgctagcacactgatttcac | 12 |
| | ggaatccttgtgcgcctgttttacaccccctccccccaactcagtaacttagaagttgtaaacaca | |
| | cgtggccaacagcgggcgtagcgcaccagctatgtcttggtcaagcacttctgtttccccggac | |
| | agagtatcaataagctgcttgcgcggctgaaggagaaaatgttcgttacccggtcaactacttcg | |
| | agaaacctagtagcatcatgaatgttgcgaggcgtttcgctcagcacttcccccgtgtagatcag | |
| | gtcgatgagtcactgcactccccacgggcgaccgtggcagtggctgcgttggcggcctgccc | |
| | atggggtgacccatgggacgcttcaatacggacatggtgtgaagagtctattgagctagttgata | |
| | atcctccggcccctgaatgcggctaatcctaactgcggagcacataccctcaacccagggggc | |
| | ggtgtgtcgtaacgggcaactctgcagcggaaccgactactttgggtgtccgtgtttccttttata | |
| | cttatactggctgcttatggtgacaattgaagaattgttaccatatagctattggattggccatccg | |
| | gtgactaacagagctattatatatttatttgttggatttgttccattaacatacacatctttcagcacttt | |
| | acacctcattttacaatacaatacaagaaa | |
| iEV71 IRES | ttaaaacagcctgtgggttgcacccactcacagggcccactgggcgcaagcactctggtacct | 13 |
| | cggtacctttgtgcgcctgttttacaccccccccccagtgaaacttagaagcagcaaaccacgat | |
| | caatagcgggcataacgctccagttatgtcttgatcaagcacttctgtttccccggactgagtatc | |
| | aatagactgctcgcgcggttgaaggagaaaacgttcgttatccggctagctacttcgggaaacc | |
| | tagtaacaccatgaaagttgcggagagcttcgttcagcactcccccagtgtagatcaggtcgat | |
| | gagtcaccgcattccccacgggcgaccgtggcggtggctgcgttggcggcctgcccatggg | |
| | gtaacccatggggcgctctaatacggacatggtgtgaagagtctactgagctagttagtagtcct | |
| | ccggcccctgaatgcggctaatcccaactgcggagcacacgcccacaagccagcgggtagc | |
| | gtgtcgtaacgggtaactctgcagcggaaccgactactttgggtgtccgtgtttctttttatctttat | |
| | actggctgcttatggtgacaattaaagaattgttaccatatagctattggattggccatccggtgtg | |
| | caacagagcaattgtttacctattcattggtttcgtaccattaaccttgaagtctgtgaccaccctta | |
| | actacatcttgacccttaacacagctaaac | |
| iEV-B107 IRES | ttaaaacagcctgtgggttgttcccacccgcagggcccactgggcgctagcacactggtatccc | 14 |
| | ggtacccttgtgcgcctgttttatataccctcccccttatgtaacttagaagtatgattcaaacggtc | |
| | gacaggcggctcagtgcaccaactgagtcatgaccaagcacttctgttaccccggactgagtat | |
| | caataagctgttcacacggctgaaggagaaaacgttcgttacccggccaattacttcgagaaac | |
| | ctagtaccaccatgaaggttgcgcagtgtttcgctccacacaaccccagtgtagatcaggtcgat | |
| | gagtcaccgcattccccacgggcgaccgtggcggtggctgcgttggcggcctgcccatggg | |
| | gcaacccatgggacgcttcaatactgacatggtgtgaagagtctattgagctaattggtagtcct | |
| | ccggcccctgaatgcggctaatcccaactgtggagcagatactcacaaaccagtgagcggtct | |
| | gtcgtaacgggcaactccgcagcggaaccgactactttgggtgtccgtgtttctttttattcttaca | |
| | ttggctgcttatggtgacaattgacaaattgttaccatatagctattggattggccatccggtgaca | |
| | aacagagctattgtttacttgtttgttggtttcataccattaaattacaaggtcttagaaactctcaact | |
| | ttattttgacactcaatacagcaaa | |
| iHRV-A9 IRES | ttaaaactggatctgggttgttcccactcagatctcccacgtggagtagtacactattattacggta | 15 |
| | atcttgtacgccagttttataatccccttacccaagtaacttagaagataaacacaaagaccaata | |
| | ggagatgatcatccagatcatcataggtcaagcacttctgtttccccggtcaaggttgatatgctc | |
| | taacagggcaaaaacagctgagatcgttatccgcaaagcgcctacgcaaagcctagtaactat | |
| | ctggaagttgcttggttggtcgctccgccatatcccatggtagacctggcagatgaggctagaa | |
| | attccccactggtgacagtgttctagcctgcgtggctgcctgcacaccctctgggtgtgaaacca | |
| | agtaatggacagggtgtgaagagccccgtgtgctcgctttgagtcctccggcccctgaatgtgg | |
| | ctaaccttaaccctgcagctagggcacacaatccagtgtgtatctagtcgtaatgagcaattgcg | |
| | ggatgggaccaactactttgggtgtccgtgtttcttgtttttcttttatgtttgcttatggtgacaatata | |
| | tagtaatatatrttggcatc | |
| iHRV-A21 IRES | ttaaaactgggtccaggttgttcccacctggatctcctattgggagttgtactctattattccggtaa | 16 |
| | ttttgtacgccagttttatctccccttccccaattgcaacttagaagtttatcaatatgaccaataggc | |
| | ggtagttagccaaactaccagaggtcaagcacttctgtttccccggtcaaagttgatatgctcca | |
| | acagggcaaaaacaactgagatcgttatccgcaaagtgcctacgcaaagcctagtaacaccttt | |
| | gaagatttatggttggtcgttccgctatttcccatagtagacctggcagatgaggctagaaattcc | |
| | ccactggcgacagtgttctagcctgcgtggctgcctgcgcaccccttgggtgcgaagccatac | |
| | attggacaaggtgtgaagagccccgtgtgctcactttgagtcctccggcccctgaatgtggcta | |
| | accttaaccctgcagctagtgcatgtaatccaacatgttgctagtcgtaatgagcaattgcggga | |
| | cgggaccaactactttgggtgtccgtgtttcactttttccttttaatattgcttatggtgacaatatata | |
| | taaacatatatattgacacc | |
| iHRV-A57 IRES | ttgtttgatgccagttttatctccccccccaattgcaacttagaagatgtacacaaagaccaatagg | 17 |
| | cagtggtcacccagaccactgaaggtcaagcacttctgtctccccggtcaaagttgatatgctcc | |
| | aacagggcaaaaacaacttagatcgttatccgcaaagtgcctacgcaaagcttagtaccatcttt | |
| | gagagcctatggttggtcgctccactgaaccccacagtagacctggcagatgaggctagaaga | |
| | cccccactggcgacagtgttctagcctgcgtggctgcctgcacacccttacgggtgtgaagcc | |
| | atatgtttgacaaggtgcgaagagccccgtgtgctcactttgagtcctccggcccctgaatgtgg | |
| | ctaaccttaaccctgcagctagtgcacacaagccagtgtgttgctagtcgtaatgagcaattgcg | |
| | ggatgggaccaactactttgggtgtccgtgtttcactttttacctttattttgcttatggtgacaatata | |
| | tatagtatatatattggcacc | |
| iHRV-A89 IRES | ttaaaactgggagtgggttgttcccactcactccacccatgcggtgttgtactctgttattacggta | 18 |
| | actttgtacgccagtttttcccacccttccccataatgtaacttagaagtttgtacaatatgaccaat | |
| | aggtgacaatcatccagactgtcaaaggtcaagcacttctgtttccccggtcaatgaggatatgc | |
| | tttacccaaggcaaaaaccttagagatcgttatccccacactgcctacacagagcccagtaccat | |
| | ttttgatataattgggttggtcgctccctgcaaacccagcagtagacctggcagatgaggctgga | |
| | cattccccactggcgacagtggtccagcctgcgtggctgcctgctcacccttcttgggtgagaa | |
| | gcctaattattgacaaggtgtgaagagccgcgtgtgctcagtgtgcttcctccggcccctgaatg | |
| | tggctaaccttaaccctgcagccgttgcccataatccaatgggtttgcggtcgtaatgcgtaagt | |
| | gcgggatgggaccaactactttgggtgtccgtgtttcctgtttttcttttgattgcattttatggtgac | |
| | aatttatagtgtatagattgtcatc | |
| iHRV-A100 | ttaaaactgaatccagattgttcccatctggatttcctacatggagttgtactctattattccggtaatt | 19 |
| IRES | ttgtacgccagttttatcaccccttcccccgtaacttagaagtttgaaacaaaagaccaataggag | |
| | gtaactatccaagttactataggtcaagcacttctgtttccccggtcaaagttgatatgctccacca | |
| | gggcaaaaacaattgagatcgttatccgcaaagtgcctacgcaaagcctagtagtatcttgaaa | |
| | agcatgtggttggttgctccgctgtaccccacagtaaacctggcagatgaggctagaagttccc | |
| | cactggtgacagtgttctagcctgcgtggctgcctgcgcactctttgagtgcgaagccatatgttt | |
| | gacaaggtgtgaagagccccgtgtgctcactttgagtcctccggcccctgaatgtggctaacctt | |
| | aaccctgcagctagtgcatgcaatccagcatgtggctagtcgtaatgagcaattgcgggatggg | |
| | accaactactttgggtgtccgtgtttcacttttttccttttataattgcttatggtgacaatatatagtga | |
| | tatatattgacacc | |
| iHRV-B4 IRES | ttaaaacagcggatgggtttcccaccatccgacccactgggtgtagtgctctggtattttgtacctt | 20 |
| | tgcacgcctgtttcccatttgtacccttccttaatctccttcccccgtaacgttagaagttttggaattt | |
| | taaagtacaataggaagcgccacatccagtggtgttgcgtacaaatacttctgtttacccggagc | |
| | gaggtataggttgtacccacggccaaaagcctttaaccgttatccgccaatcaactacgtaacg | |
| | gctagtatcatcttgcttttgatttggtgttcgatcaggtggtatcccccactagtctggtcgatgag | |
| | gctaggaattccccacgggcgaccgtgtcctagcctgcgtggcggccagcccagcttttgctg | |
| | ggacgccttttcaaagacatggtgtgaagacctgcatgtgcttggttgtgagtcctccggcccct | |
| | gaatgcggctaaccttaaccctggagcccagcagcataatccaatgttgtttgggtcgtaatgag | |
| | caattccgggacgggaccgactactttgggtgtccgtgtttccttttattcttacattgtcttatggtc | |
| | acagcatatatattatatatactgtgatc | |
| iHRV-B17 IRES | tttaaacagcggatgggttccccaccatccgacccactgggtgtagtactctggtattttgtacctt | 21 |
| | tgtacgcctgtttctcccctacctcccaacctaaacaatcctggtaacttagaagacttaaatcatc | |
| | gtacaataggtgctgtcacatccagtgacggctagtacaagcacttctgtttccccggagcgga | |
| | gtataaatggccaccgctgtcaaaagctcttaaccgttatccgccaattaactacgcaacggcta | |
| | gtaacatcttgttatttttagggcgttcgatcaggtgagtaaacccctcactagtctggtcgatgag | |
| | gctgagaattccccacgggcgaccgtgtctcagcctgcgtggcggccagcccagctaatgct | |
| | gggacgccttaattgtgacatggtgtgaagacccacgtgtgcttaattgtgagtcctccggcccc | |
| | tgaatgcggctaacctaaaccctggagccttgagacacaatccagtgttggcaaggtcgtaatg | |
| | agtaattccgggacgggaccgactactttgggtgtccgtgtttccttttattttcaaattgttcttatg | |
| | gtcacaatataagtaatatattgtgatc | |
| iHRV-B26 IRES | ttaaaacagcgggtgggtatcccaccacccgacccactgggtgttgtacactggtattttgtacct | 22 |
| | ttgtatgcctgtttgcacctccccaccccttccaattacccttacccgaattrtattatgcggtaacat | |
| | tagaagaagtgaacacagtgcaataggacgtatcacatccagtgatataaagcacaagcaattc | |
| | ttgttccccggagctggatatagactgctaacgtggttgaaggtccttaaccgttatccgccaacc | |
| | aactacgagacggctagtaatatcatgtttgtctttgagcgttcgatcaggtgaattccccattcac | |
| | tagtttggtcgatgaggctgagaactccccacaggtgactgtgtctcagcctgcgtggcggcca | |
| | acccagccacggctgggacgcccactgatagacatggtgtgaagacccaattgtgcttggttgt | |
| | gactcctccggcccctgaatgcggctaacctcaaccccggagccttgtagtgtaagccaacac | |
| | atacaaggtcgtaatgggcaactctgggacgggaccgactactttgggtgtccgtgtttcctttat | |
| | ttttatcttttgtgtcttatggttacaagtattgattgtaacc | |
| iHRV-B37 IRES | tttaaacagcggatgggtatcccaccatccgacccactgggtgtagtactctggtattttgtacctt | 23 |
| | tgtacgcctgttgttcctaatgtacccaccctaaaacttcctacccaagtaacgttagaagtttcatc | |
| | aacaagtacaataggaagcatcacatccagtggtgttttgtacaagcacttctgtttccccggag | |
| | cgaggtataggctgtacccactgccgaaagcctttaaccgttatccgccaaccaactacgtaaa | |
| | agctagtatcatcatgttttaaaataggcgttcgatcaggtggatcccccctccactagtttggtcg | |
| | atgaggctaggaactccccacgggtgaccgtgtcctagcctgcgtggcggccaacccagcttc | |
| | tgctgggacgcctttttatggacatggtgtgaagactcgcatgtgcttggttgtgactcctccggc | |
| | ccctgaatgcggctaaccttaaccccggagccctgtgttgcaatccagtaacattagggtcgtaa | |
| | tgagcaatttcgggacgggaccgactactttgggtgtccgtgtttctcatttttcttattattgtcttat | |
| | ggtcacagcatatatataacgtatatactgtgatc | |
| iHRV-B92 IRES | ttaaaacagcggatgggtatcccaccatccggcccactgggtgtagtactctggtacattgtacc | 24 |
| | tttgtacgcctgttttccccctcttgtacccgcccttcaagctccttgcccaagtaacgttagaagtt | |
| | tgaacattggtacaataggaagcatcacatccagtggtgtactgtacaaacacttctgttgcccc | |
| | ggagcgaggtatagatggtccccaccgtcaaaagcctttaaccgttatccgccaatcaactacg | |
| | taatggctagtagcaccttggatttaagttggcgttcgatcaggtggtaacccccactagtttggt | |
| | cgatgaggctaggaattccccacgggtgaccgtgtcctagcctgcgtggcggccaacccagc | |
| | atccgctgggacgccaatttaatgacatggtgtgaagacctgcatgtgcttgattgtgagtcctcc | |
| | ggcccctgaatgcggctaaccctaaccccggagccttgcagcacaatccagtgttgttaaggtc | |
| | gtaatgagcaattctgggatgggaccgactactttgggtgtccgtgtttcttatttttcttgaatttttc | |
| | ttatggtcacagcatatatacattatatactgtgatc | |
| IHRV-C3 IRES | ttaaagctggatcatggttgttcccaccatgattacccacgcggtgcagtggtcttgtattacggta | 25 |
| | catttccataccagttttatacaccccaccccgaaactcatagaagtttgtacacaatgaccaata | |
| | ggtggtggccatccaggtcgctaatggtcaagcacttctgtttccccggcacccttgtatacgctt | |
| | cacccgaggcgaaaaatgaggttgtcgttatccgcaaagtgcctacgaaaagcctagtaacac | |
| | tttgaaaacccatggttggtcgctcagctgtttacccaacagtagacctggcagatgaggctaga | |
| | cattccccaccagcgatggtggtctagcctgcgtggctgcctgcacaccctgccgggtgtgaa | |
| | gccagaaagtggacaaggtgtgaagagcctattgtgctcactttgagtcctccggcccctgaat | |
| | gtggctaaccctaaccccgtagctgttgcatgtaacccaacatgtatgcagtcgtaatgggcaac | |
| | tatgggatgggaccaactactttgggtgtccgtgtttcctgttttactttttcattgcttatggtgacaa | |
| | ttgtatctgatacacttgttacc | |
| IHRV-C11 IRES | ttaaaactggatacaggttgttcccacctgtatcacccaagtggtgtggtgctcttgtatttcggta | 26 |
| | cgtttgcacgccagtttgctaccccttcccttttacgtaacttagaagtttacacaaagaccaatag | |
| | gcggtggtaaatccataccactaacggtcaaatacttctgtttccccggcatgcgaggaatagg | |
| | ctccaaaagggctgaagccactagtgtcgttatccgcattggtactacgcaaagcctagtattac | |
| | cttgaaaatttcttggctggtcgctccaccagataccccactggtagacctggcagatgaggca | |
| | ggacttaccccactggcgacagtggtcctgcctgcgtggctgcctgcacaccccttacggggt | |
| | gtgaagcccagaaacagacaaggtgtgaagagccccgtgtgctactagtgagtcctccggcc | |
| | cctgaatgcggctaatcttaccccacagctgttgcacgcaaaccagcgtgtatgcagtcgtaatg | |
| | agcaattgtgggatggaaccgactactttgggtgtccgtgtttctttttattcctattatttgcttatgg | |
| | tgacaatattgatattatcagtgttgtcatc | |
| iHRV-C54 IRES | ttaaaacagctgtgtggttgttcccaccaccaggcacactgtgcgttgtacactgggattccggt | 27 |
| | cactttgtacgcctgtttgctatcccccccaacttatgtaatttagaagatgtacacaacgcccatta | |
| | ggatgcggccaaaccaggtccgcttagggcaagcacttctgtttccccgggtgtgtgaatagac | |
| | tctaacagggttgaagctgtagcactcgttatccgcgcaactactacgcgaatgttagtagcatc | |
| | ctgtgttgcatttgggatttcgctccgcagaaaaccccatctgtagattagggcaatgaggctac | |
| | acataccccactggcgacagtggtgtagcctgcgtggtgccctacccaggccttcttggcctgg | |
| | gattccacttacaagacagggtgtgaaggcactagtgtgctagttgtgagtcctccggcccctg | |
| | aatgcggctaatcttaaccccgtagcccccgcaagtaaaccaacttgtaggtggtcgtaatgagt | |
| | aattacgggatggaaccgactactttgggtgtccgtgtttccttttattctttatatttgcatcctatgg | |
| | ttacaacataagtaatc | |
| iCVB1 IRES | ttaaaacagcctgtgggttgttcccacccacaggcccattgggcgctagcactctggtatcacg | 28 |
| | gtacctttgtgcgcctgttttacatcccctccccaaattgtaatttagaagtttcacacaccgatcatt | |
| | agcaagcgtggcacaccagccatgttttgatcaagcacttctgttaccccggactgagtatcaat | |
| | agaccgctaacgcggttgaaggagaaaacgttcgttacccggccaactacttcgaaaaaccta | |
| | gtaacaccatggaagttgcggagtgtttcgctcagcactaccccagtgtagatcaggtcgatga | |
| | gtcaccgcgttccccacgggcgaccgtggcggtggctgcgttggcggcctgcctacgggga | |
| | aacccgtaggacgctctaatacagacatggtgcgaagagtctattgagctagttggtaatcctcc | |
| | ggcccctgaatgcggctaatcctaactgcggagcacataccctcaaaccagggggcagtgtgt | |
| | cgtaacgggcaactctgcagcggaaccgactactttgggtgtccgtgtttcattttattcctatact | |
| | ggctgcttatggtgacaattgacaggttgttaccatatagttattggattggccatccggtgactaa | |
| | cagagcaattatatatctctttgttgggtttataccacttagcttgaaagaggttaaaacactacatc | |
| | tcatcattaaactaaatacaacaaa | |
| iCVB5 IRES | ttaaaacagcctgtgggttgttcccacccgcagggcccactgggcgccagcactctggtatcac | 29 |
| | ggtacctttgtgcgcctgttttaaaaccctctccccaatttgaaacttagaagcaatacacctcgat | |
| | caatagtaggcatgacacgccagccatgtcttgatcaagcacttctgtttccccggactgagtat | |
| | caataaactgcttgcgcggtcgaaggagaaaacgtccgttacccgactaactacttcgagaaac | |
| | ccagtaacaccatggaaattgcggagtgtttcactcagcacattcccagtgtagatcaggtcgat | |
| | gagtcaccgcattccccacgggtgaccgtggcggtggctgcgctggcggcctgcccatggg | |
| | gcaacccatgggacgcttcaatatggacatggtgtgaagagtctattgagctagttagtagtcct | |
| | ccggcccctgaatgcggctaatcctaactgcggagcacgtgcctccattccagggggtggcgt | |
| | gtcgtaacgggcaactctgcagcggaaccgactactttgggtgtccgtgtttcttttaattttatact | |
| | ggctgcttatggtgacaattgaaagattgttgccatatagctattggattggccatccggtatccaa | |
| | cagagcaattgtgtacctttttgttggatttgtaccacttaccagaacaagttttcatacactgtgtta | |
| | cattattagactaaacacagaaaa | |
| iEchoV1 IRES | ttaaaacagcctgtgggttgttcccacccacagggcccactgggcgttagcacactggtatcac | 30 |
| | ggtacctttgtgcgcctgttttatactcccccccctaaggaaactttagaagcaaagcaattgtgat | |
| | caatagtgggtatggcacaccagtcatatcttgatcaagcacttctgttcccccggacttagtacc | |
| | aatagactgctcaagcggttgaaggggaaaacgttcgttatccggccaactacttcgagaaacc | |
| | tagtagcaccatgaaagttgcggagtgtttcgctcagcacttcccccgtgtagatcaggctgatg | |
| | agtcaccgtattccccacgggcgaccgtgacggtggctgcgttggcggcctgcccatggggt | |
| | aacccatgggacgctctaaaacagacacggtgcgaagagtctattgagctagttggtagtcctc | |
| | cggcccctgaatgcggccaatcctaactgcggagcacatactcccaatccagggagcagtgt | |
| | gtcgtaatgggtaactctgcagcggaaccgactactttgggtgtccgtgtttccttttattctcacat | |
| | tgactgcttatggtgacaattgaaagattgttaccatatagctattggattggtcatccggtgagca | |
| | atagagctattgtttatcaatttgttggatttgtaccactcaacttttctgttttgagaacactcaacta | |
| | catcttactgctaaacacatcaaa | |
| iEchoV-E11 | ttaaaacagcctgtgggttgtacccacccacagggcccactgggcgctagcacactggtatca | 31 |
| IRES | cggtacctttgtgcgcctgttttatacccccttcccgcaaccgcaaatttagaagcaaagctaacc | |
| | cgatcgatagcggatgcgcatgccagccgcattttgatcaagtacttctgtttccccggaccgag | |
| | tatcaatagactgctcacgcggttgaaggagaaaacgtccgttacccgaccaactacttcgaga | |
| | aacctagtaacatcatgaatgttgcagggcgtttcgatcagcacgaccctggtgtagatcaggct | |
| | gatgagtcaccgcattccccacgggtgaccgtggcggtggctgcgttggcggcctgcctatgg | |
| | ggtgacccataggacgctctaatacggacatggtgcgaagagtctattgagctagttggtagtc | |
| | ctccggcccctgaatgcggttaatcctaactgcggacgacatacccctaatccaaggggcagt | |
| | gtgtcgtaacgggcaactctgcagcggaaccgactactttgggtgtccgtgtttccttttatttttat | |
| | actggctgcttatggtgacaatctcagagttgttaccatatagctattggattggccatccggtga | |
| | gcaacagagctgtcatttatcagtttgttggctttatacctctaaatcacacggttttttttttttggaac | |
| | gcttgtattcatcttaaccctcaataaggcaaa | |
| iEchoV-E15 | ttaaaacagcctgtgggttgtacccacccacagggcccactgggcgctagcactctggtattac | 32 |
| IRES | ggtacccttgtgcgcctgttttatataccccgccccaagcaaacgctagatgtaacgcacttatga | |
| | tcaatagcaggcgtggcactccagccacgttatgatcaagcacttctgtctccccggaccgagt | |
| | atcaatagactgctcacgcggtcgaaggagaaaacgttcgttacccgaccagctacttcgaga | |
| | aacctagtaactccatggaggttgcagagtgtttcgttcagcacttcccccgtgtagatcaggct | |
| | gatgagtcaccgcgttcctcacgggcgaccgtggcggtggctgcgttggcggcctgcctatgg | |
| | gtatacccataggacgctctaatactgacatggtgcgaagagtctattgagctagttggtagtcct | |
| | ccggcccctgaatgcggctaatcctaactgcggagcacatacccccaacccagggggcagtg | |
| | tgtcgtaacgggtaactctgcagcggaaccgactactttgggtgtccgtgtttccttttattcttaca | |
| | ttggctgcttatggtgacaattgagagattgttaccatatagctattggattggccatccagtgact | |
| | aacagagctattatttacctttttgttggcttcgtatcacttggtttaaaagaggttagtactttatattg | |
| | cattatattactaaacacgagaaa | |
| iCVA20 IRES | ttaaaacagctctggggttgtacccaccccagaggcccacgtggcggctagtactccggtatta | 33 |
| | cggtacccttgtacgcctgttttatactccctccctcgtaacttagaagcacaaaaccaagttcaat | |
| | agaagggggtgcaaaccagtaccaccacgaacaagcatttctgtttccccggtgatgttgtata | |
| | gactgcccgcgcggttgaaagcaacggatccgttacccgctcaagtacttcgagaagcctagt | |
| | attaccttggaatcttcgatgcgttgcgttcagcactcgaccctggagtgtagcttaggctgatga | |
| | gtctggacgtccctcaccggtgacggtggtccaggctgcgttggcggcctacctatggctaac | |
| | gccataggacgctagttgtgaacaaggtgtgaagagcctattgagctacttgagagtcctccgg | |
| | cccctgaatgcggctaatcctaaccatggagcaggcggtcacagaccagtgactagcttgtcgt | |
| | aatgcgcaagtctatggcggaaccgactactttgggtgtccgtgtttccttttatttttattatggctg | |
| | cttatggtgacaatcattgattgttatcataaagcgaattggattggccatccggtgaaagcgaga | |
| | cttactatttacttacttgttggacttaccacacttaatacatttattctaggtgtcacttgtatagcaat | |
| | tagaatcaaacagttgcatcata | |
| iEV-D94 IRES | ttaaaacagcctgtgggttgttcccaccccagaggcccacgtggcggccagtactctggtatca | 34 |
| | cggtacctttgtacgcctgttttatatccccttcccccgcaacttagaagaaaacaaatcaagttca | |
| | ctaggagggggtacaaaccagtaccaccacgaacaagcacttctgtttccccggtgatgtcgta | |
| | tagactgtaaccacggttgaaaacgattgatccgttatccgctcttgtacttcgaaaagcccagta | |
| | tcaccttggaatcttcgatgcgttgcgctcagcactcaaccccagagtgtagcttaggtcgatga | |
| | gtctggacactcctcaccggcgacggtggtccaggctgcgttggcggcctacctgtggtccaa | |
| | agccacaggacgctagttgtgaacaaggtgtgaagagcctattgagctacaagagaatcctcc | |
| | ggcccctgaatgcggctaatcctaaccacggagcaagggtacacaaaccagtgtatatcttgtc | |
| | gtaacgcgcaagtctgtggcggaaccgactactttgggtgtccgtgtttccttttgtttttatcatgg | |
| | ctgcttatggtgacaatctaagattgttatcatatagctgttggattggccatccggtaatttattgag | |
| | atttgagcatttgcttgtttcttcaacaatttcacctattcattgcatttcagcagtcaaa | |
| iSimianEV-A | gagtgttcccacccaacaggcccactgggtgttgtactctggtattacggtacctttgtacgccta | 35 |
| IRES | ttttatttcccccccctttttgaaacttagaagttaataataaacacgctcactaggtgcactacatcc | |
| | agtagtgtaatgagcaagcacttctgtctyccccgggagggatatatggtacgctgtgcaaacg | |
| | gcggaaattaatcctaccgttaaccgcccacctactccgagaagcctagtacctaattggatttat | |
| | caatggagttgcgctcagcaggtgaccctgacctgccagctccggctgatggacctgggctttc | |
| | cccacaggcgactgtggcccaggtcgcgtggcggccggcccacccccctgggtgggacgc | |
| | cttgataatgacaaggtgggaagagcctattgggctagctggtttcctccggcctcctgaatgcg | |
| | gctaaccttaaccccagagcatatggtagcaacccagctactagtatgtcataatgcgtaagtct | |
| | gggatgggaccgactactttggagagtccgtgtttctattgtttctttaatcaatcttatggtgacaa | |
| | tttatagtgccctgagtattgattggttgttgcttttgacaattattgagacatcacatagacata | |
| iSimianA5 | ttaaaatagcctgtgggttgctcccacccacagggcccaagtggcgtagtacttggtattccggt | 36 |
| IRES | acctttgtacacctatttacaaaccctaccccttgtaaccttagaagcaattatttaaccgctcacta | |
| | gggggtgtgctatccaagcacatcaagagcaagcacttctgtctccccgggaggggctaatgg | |
| | tacgctgtgcccacggcggaaatgagccctaccgttaaccggcagtctacttcgggaagccca | |
| | gtaactacattgaaactttgaggcgttacactcagcacataaccccaatgtgtagttctggtcgat | |
| | gagccttggcatcccccacaggcgactgtggccaaggctgcgttggcggccagcctgcgga | |
| | ccaaaagtccgtaggacgcctaattgtggacatggtgtgaagagcctactgagctagactgtag | |
| | tcctccggcccctgaatgcggctaatcctaaccctggagcatccgcgtgcaacccagtacgta | |
| | gggtgtcgtaatgcgtaagtctgggatggaaccgactactttgggtgtccgtgtttcttgtttttcat | |
| | actgggtcgcttatggttacaactaattgttgtaatcattggcagtgcgcgctgaccacgcgattat | |
| | tgatatttccatttgttggatactccaatagtgtcaactcatatacacaacttttaccactgatcaaga | |
| | taaaa | |
| iSimian V4 | ttaaaatagctgctgggttgttcccatccagcaggcccactgggcgtgatactctggtattgcggt | 37 |
| IRES | acctttgtatgcctattttacctcccttccccctgaaacttagaagaaagaaacaaacacgctcact | |
| | aggtacactgcatccagcagtgtaatgagcaagcacttctgtttccccggaagggatatatggta | |
| | cgctgtgcaaacggcggaaattaatcctaccgttaaccgcccatctactccgagaagcctagta | |
| | cctaattgaacttatcaatggagttgcgctcagcaggtgaccctgacctgccagctccggctgat | |
| | ggacctgggcatcccccacaggcgactgtggcccaggtcgcgtggcggccggcccactcttt | |
| | tgagtgggacgccttgataatgacaaggtgggaagagcctattgggctagctggtttcctccgg | |
| | cctcctgaatgcggctaaccctaaccccggagcatatggtggcaacccagccactggtatgtc | |
| | gtaacgcgtaagtctgggatgggaccgactactttggagagtccgtgtttccattattttctttattg | |
| | aatcttatggtgacaaattgggagttactcgggtgacgattgattattacttctgaaaacgtagaga | |
| | tatagaatcaacaca | |
| iSwine | ttaaaacagcctgtgggttgttcccacccacagggcccactgggcgctagcacactggtatcac | 38 |
| Vesciular | ggtacctttgtgcgcctgtttgacttaccctccccaaacgcaacttagaagcacaacttaaatggt | |
| IRES | caataggcggctcagtatgccaactgagtctcgatcaagcacttctgttaccccggactgagtac | |
| | caataggctgctcacgcggctgaaggggaaaccgttcgttacccgactaactacttcgagaaa | |
| | cctagtaccaccatgaaagttgcgcagcgtttcgctccgcacaaccccagtgtagatcaggtcg | |
| | atgagtcaccgcaaaccccacgggcgaccgtggcggtggctgcgctggcggcctgcccatg | |
| | gggcaactcatgggacgcttcaatactgacatggtgcgaagagtctattgagctagttggtagtc | |
| | ctccggcccctgaatgcggctaatcctaactgcggagcagatacccacgcaccagtgggcag | |
| | tctgtcgtaatgggcaactctgcagcggaaccgactactttgggtgtccgtgtttccttttgttctta | |
| | tactggctacttatggtgacaattgagagattgtaaccatattgctattggattggccacccggcg | |
| | acgaatagaacagttgcttacctgtttgttagtctcgtatcactgaactacaaatccttaaacaccct | |
| | ttaatttcgtcataacgctcaatacgttaaa | |
| IHRV-B3_ | ttaaaacagcggatgggtatcccaccatccgacccactgggtgtagtactctggtactttgtacct | 39 |
| SC2220 IRES | ttgtacgcctgttcttcccattgtacccttcctaaacttccaacccaagtaacgttagaagctcaact | |
| | ttcagtacaataggaagcaccacatccagtggtgtttagtacaagcacttctgtttccccggagc | |
| | gaggtataggctgtacccactgccaaaagcctttaaccgttatccgccaaccaactacgtaaaa | |
| | gctagtaacatcatgtttgtaaatgggtgttcgatcaggtggatttcccctccactagtttggtcgat | |
| | gaggctaggaattccccacgggtgaccgtgtcctagcctgcgtggcggccaacccagcgtat | |
| | gctgggacgcctttttatagacatggtgcgaagactcgcatgtgcttggttgtgagtcctccggc | |
| | ccctgaatgcggctaaccctaaccctggagccttgagtcacaatccagtgatggcaaggtcgta | |
| | atgagcaattccgggatgggaccgactactttgggtgtccgtgtttctcatttttcttattattgtctta | |
| | tggtcacagcatatatagtatatatactgtgatc | |
| iHRV-B5 IRES | ttaaaacagcgggtgggtatcccaccacccgacccactgggtgtggtacactggtactttgtac | 40 |
| | ctttgtatgcctgtttgcttccactcccctttacctaatattcttccccaagtatattttgcggtaacgtt | |
| | agaagaagcaaagttagtgcaataggatgtatcacaaccagtggtataaagcacaagcaattct | |
| | tgttccccggagctggatatagactgctaacgtggtcaaaagtccttaaccgttatccgccagct | |
| | aactgcgagaagactagtaacaccatgtttgtttttagacgttcgatcaggtgaataccccattca | |
| | ctagtttggtcgatgaggctgagaataccccacaggtaactgtgtctcagcctgcgtggcggcc | |
| | aacccgactcatgtcgggacgcctactaatagacatggtgtgaagatcctattgcgcttagttgt | |
| | gagtcctccggcccctgaatgcggctaaccctaaccccggagccttgtggtgtaaaccaacatt | |
| | tgcaaggtcgtaatgagtaattctgggatgggaccgactactttgggtgtccgtgtttcctttattttt | |
| | attgtgtgtgtcttatggttacaaatacaatttgtaatc | |
| IHRV-B6 IRES | ttaatacagcggatgggtatcccaccatccgacccactgggtgtagtactctggtattatgtacct | 41 |
| | ttgtacgcctgttcttcccattgtacccttcccaaatttccaacccaagtaacgttagaagtttgaca | |
| | ttaaagtacaataggaggcaccacatccagtggtgttttgtacaagcacttctgtttccccggagc | |
| | gaggtataggctgtacccactgccaaaaacctttaaccgttacccgccagccaactacgtaaaa | |
| | gctagtaacatcttgtttgtaacttggcgttcgatcaggtggattccccctccactagtttggtcgat | |
| | gaggctaggaactccccacgggtgaccgtgtcctagcctgcgtggggccaacccagctctt | |
| | gctgggacgcccttttatagacatggtgtgaagactcgcatgtgcttggttgtgattcctccggcc | |
| | cctgaatgcggctaaccctaaccctggagccttgtgttacaaaccagtaatattaaggtcgtaat | |
| | gagcaattccgggacgggaccgactactttgggtgtccgtgtttctcatttttcttattattgtcttat | |
| | ggtcacagcatatttgttaaatatactgtgatc | |
| iHRV-B14 IRES | tactctggtattatgtacctttgtacgcctgtttcttccctacaaccccttcctaaaactcccacccat | 42 |
| | gaaacgttagaagcttgacatttaagtacaataggtggcaccacatccagtggtgtctacgtaca | |
| | agcacttctgtttccccggagcgaggtataggctgtacccactgccaaaagcctttaaccgttat | |
| | ccgccaaccaactacgtaacagctagtatcatcttgttcttcactggacgttcgatcaggtggatt | |
| | ccccctccactagtttggtcgatgaggctaggagctccccacgggtgaccgtgtcctagcctgc | |
| | gtggcggccaacccagctcatgctgggacgcccttttaaggacatggtgtgaagactcgcatgt | |
| | gcttggttgtgagtcctccggcccctgaatgcggctaaccttaaccctggagccttatgccacga | |
| | accagtggttgtaaggtcgtaacgagcaattccgggatgggaccgactactttgggtgtccgtg | |
| | tttcctatttttctttatattgtcttatggtcacagcatatatataagtatatactgtgatc | |
| IHRV-B27 IRES | ttaaaacagcggatgggtatcccaccatccgacccacagggtgtagtgctctggtattttgtacct | 43 |
| | ttgcacgcctgtttccccattgtacccctccttaaatttcctccccaagtaacgttagaagtttaagg | |
| | aaacaaatgtacaataggaagcatcacatccagtggtgttatgtacaagcacttctgtttccccgg | |
| | agcgaggtataagtggtacccaccgccgaaagcctttaaccgttatccgccaatcaactacgta | |
| | atggctagtattaccatgtttgtgacttggtgttcgatcaggtggttccccccactagtttggtcgat | |
| | gaggctaggaactccccacgggtgaccgtgtcctagcctgcgtggcggccaacccagcttttg | |
| | ctgggacgcctttttacagacatggtgtgaagacctgcatgtgcttgattgtgagtcctccggccc | |
| | ctgaatgcggctaaccttaaccccggagccttgcaacataatccaatgttgttgaggtcgtaatg | |
| | agtaattctgggatgggaccgactactttgggtgtccgtgtttccttttattctttatattgtcttatggt | |
| | cacagcatatatagcatatatactgtgatc | |
| iHRV-B35 IRES | ttaaaacagcggatgggttccctaccatccgacccactgggtgtagtactctggtactctgtacct | 44 |
| | ttgtacgcctgtttatccactacccttttcctaaattttccacccgtgtaaccttagaagcaaacaaat | |
| | tgtacaatagggtgcatcacaaccagtgatgtacagtacaagcacttctgtttccccggagcgg | |
| | ggtatagacggtcaccactgtcaaaagcccttaaccgttacccgccaaccaactacgtaacgg | |
| | ctagtaacaccatgaatttgtcttgacgttcgatcaggtggtaaccccccactagtttggtcgatg | |
| | aggctgagaactccccacgggtaaccgtgtctcagcctgcgtggcggccaacccagcttctgc | |
| | tgggacgccaattcattgacatggtgtgaagacctgcatgtgcttggttgtgattcctccggcccc | |
| | tgaatgcggctaaccctaaccccggagccacgcggcataatccaatgtcgtatgggtcgtaatg | |
| | agtaattctgggatgggaccgactactttgggtgtccgtgtttcctgtttttccaaattattcttatggt | |
| | cacagcatataaatagatatatactgtgatc | |
| iHRV-B42 IRES | ttaaaacagcgggtgggtatcccaccacccgacccactgggtgtggtacactggtactttgtac | 45 |
| | ctttgtatgcctgtttgcatccccttccctttacctaacatccctccccaagttatattttgcggtaac | |
| | gttagaagaagtgaacAtagtgcaacaggaagtatcaccaccagtgatataacgcacaagca | |
| | attcttgttccccggagctggatatagactgctaacgtggttgaaagtccttaaccgttatccgcc | |
| | aATCaactgcgaaaagactagtaacatcatgcacatttctaggtgttcgatcaggtgaaaacc | |
| | ccattcactagtttggtcgatgaggctgagagtaccccacaggcgactgtgtctcagcctgcgt | |
| | ggcggccaacccgacttatgtcgggacgcctattaatagacatggtgtgaagatcctattgcgct | |
| | tagctgtgagtcctccggcccctgaatgcggctaaccttaaccccggagtcttgtgatgtaagcc | |
| | aacatttgcaaggtcgtaatgagtaattctgggatgggaccgactactttgggtgtccgtgtttcct | |
| | ttattcttatcatttgtgtttcatggttacaagcattgtttgtaatc | |
| iHRV-B48 IRES | ttaaaacagcggatgggtatcccaccatccgacccactgggtgtagtactctggtactctgtacc | 46 |
| | tttgtacgcctgtttctcctcaaccctcccctacccgttatatatctcagtaacttagaagaATtcat | |
| | taccgcacaatagggaccgttgtaaccagcaacggtcagtgcaagcacttctgtttccccggag | |
| | cggagtatagacggccactgctgtcaaaagctcttaaccgttacccgccagtATactacacaa | |
| | cagctagtaacatcttgttgttcttagggcgttcgatcaggtgagtttaccccctcactagtctggt | |
| | cgatgaggctgagaaatccccacgggtgaccgtgtctcagcctgcgtggcggccagcccagc | |
| | ttatgctgggacgccttaagtatgacatggtgtgaagactcgcgtgtgcttagctgtgagtcctcc | |
| | ggcccctgaatgcggctaacctaaaccccggagccttgaggcacaatccagtgcccgcgagg | |
| | tcgtaacgagaaattctgggacgggaccgactactttgggtgtccgtgtttccttttatctttttgat | |
| | gttcttatggttacaatatataaagtatattgtaatc | |
| iHRV-B52 IRES | ttaaaacagcggatgggtatcccaccatccgacccactgggtgtagtactctggtattatgtacct | 47 |
| | ttgtacgcctgtttctcccccccattacccctccccacatatcccagtaacttagaagaattaaatta | |
| | tcgcacaataggagctattacaaccagtaatagttagtgcaagtacttctgtttccccggagcgg | |
| | agtatagatggccaccgctgtcaaaagctcttaaccgttatccgccagtATactacgtaacgg | |
| | ctagtaacatcttgttgtgcttaaggtgttcgatcaggtgaagagacacccccttcactagtctggt | |
| | cgatgaggctgagaaatccccacgggcgaccgtgtctcagcctgcgtggcggccagcccag | |
| | cttatgctgggacgccttaagtatgacatggtgtgaagacccgcatgtgcttaactgtgagtcctc | |
| | cggcccctgaatgcggctaaccttaaccctggagccttggggtacaatccagtgctaacaaggt | |
| | cgtaacgagcaattccgggacgggaccgactactttgggtgtccgtgtttccttttatttcattattg | |
| | ttcttatggttacaatatatagagtatattgtaatc | |
| iHRV-B69 IRES | ttaaaacagcggatgggtatcccaccatccgacccactgggtgtagtactctggtactttgtacct | 48 |
| | ttgtacgcctgtttctccctcccttcccaacctaaacaatccttgtaacttagaaggtttcgattatcg | |
| | cacaatagggactattgcaaccagcaatagtcagtgcaagcacttctgtttccccggagcggag | |
| | tgtagatggccaccgctgtcaaaagctcttaaccgttatccgccagtATactacgcaacggct | |
| | agtagtgccttgttgcttttatggcgttcgatcaggcgagatttaccccctcgctagtctggtcgat | |
| | gaggctgagaattccccacgggcgaccgtgtctcagcctgcgtggcggccagcccagccttt | |
| | gctgggacgccttgattgcgacatggtgtgaagacccgcgtgtgcttaactgtgagtcctccgg | |
| | cccctgaatgcggctaacctaaaccctggagccttgaaacacaacccagtgttcgcaaggtcgt | |
| | aacgagcaattccgggacgggaccgactactttgggtgtccgtgtttcctttattctttcaattgttc | |
| | ttatggtcacaatataagtgttatattgtgatc | |
| iHRV-B70 IRES | ttaaaacagctgatgggttccccaccatccgacccaatgggtgtagtactctggtattctgtacctt | 49 |
| | tgtacgcctgtttctccccacccccccgtcctaaataatcccagtaacttagaagatttgaattatc | |
| | gtacaataggtgctgtcacatccagtgatggctagtacaagcacttctgtttccccggagcggag | |
| | tatagatggccaccgctgtcaaaagctcttaaccgttatccgccaattaactacgcaacggttagt | |
| | aacatcttgtcatttttgaggcgttcgatcaggtgagtacacccctcactagtctggtcgatgagg | |
| | ctgagaattccccacgggcgaccgtgtctcagcctgcgtggcggccagcccagcttatgctgg | |
| | gacgccttaattgtgacatggtgcgaagacccacgtgtgcttaattgtgagtcctccggcccccg | |
| | aatgcggctaacctaaaccccggagccttgagacacaatccagtgttagcaaggtcgtaacga | |
| | gcaattctgggacgggaccgactactttgggtgtccgtgtttcctgttattctcaaattgttcttatg | |
| | gtcacaatataagtaatatatatattgtgatc | |
| iHRV-B72 IRES | ttaaaacagcggatgggttctcccaccatccgacccactgggtgtagtactctggtatgatgtac | 50 |
| | ctttgtacgcctgtttattcccaaacccccacccgttattcgcccaacccctgtaacgttagaagttt | |
| | ggaacttaattgtacaatagggagcaccatatccaatggtgtaacgtacaagcacttctgtttccc | |
| | cggagcgaggtataggtggtacccactgccgaaagcctttaaccgtcatccgccaatcaacta | |
| | cgtaacagctagtaacatcttgttgttgattggacgttcgatcaggtggttcaccccccactagttt | |
| | ggtcgatgaggctgggaatcccccacgggtgaccgtgtcccagcctgcgtggcggccaacc | |
| | cagcttatgctgggacgccctttcaatgacatggtgtgaagactcgcatgtgcttgattgtgagtc | |
| | cgccggcccctgaatgcggctaaccctaaccctggagccttgcaccacaatccagtggtgtct | |
| | gggtcgtaatgagtaattctgggatgggaccgactactttgggtgtccgtgtttctcatttttctttta | |
| | attgtcttatggtcacagcatatagtattatatactgtgatc | |
| iHRV-B79 IRES | ttaaaacagcggacgggtatcccaccatccggctcactgagcgtagtactctggtattgtgtacc | 51 |
| | tttgtacgcctgttttccccaaatcccctaccctttatatctcctaccccgtaacgtaagaagttttca | |
| | aacagtacaataggaagcaccacatccagtggtgttatgtacaagcacttctgtttccccggagc | |
| | gaggtataggtggtcaccaccgccaaaagcctttaaccgttacccgccaaccaactacgtaatg | |
| | gctagtaacatcattgatttagattggcgttcgatcaggtggtatccccccactagtttggtcgatg | |
| | aggctaggaataccccacgggtgaccgtgtcctagcctgcgtggcggccaacccagcatacg | |
| | ctgggacgccagtttactgacatggtgcgaagacttgcatgtgcttggttgagagtcctccggcc | |
| | cctgaatgcggctaaccttaaccccggagcctcgcagcataatccaatgttgtagaggtcgtaa | |
| | cgagtaattctgggatgggaccgactactttgggtgtccgtgtttcctgtttttctttaaaatgtcttat | |
| | ggtcacagcatatatactgtatatactgtgatc | |
| iHRV-B83 IRES | gtacaataggttgcactacatccagtggtgtttcgtacaagcacttctgtttccccggagcggggt | 52 |
| | atagacggtacccactgtcaaaagcccttaaccgttatccgccaatcaactacgtaatggctagt | |
| | aacatcatgaaggtgagttgacgttcgatcaggtggtaacccccactagtttggtcgatgaggct | |
| | aggaatcccccacgggtaaccgtgtcctagcctgcgtggcggccaacccagcgtttgctggg | |
| | acgccaattcactgacatggtgcgaatacttgcatgtgcttgattgtgattcctccggcccctgaa | |
| | tgcggctaaccttaaccccggagccttgcagcataaaccaatgttgtggaggtcgtaatgagca | |
| | attctgggatgggaccgactactttgggtgtccgtgtttcctgtttttctcaaattgtcttatggtcac | |
| | agcatatatattgtatatactgtgatc | |
| iHRV-B84 IRES | ttaaaacagcggatgggtatcccaccatccggcccactgggcgtagtacactggtattctgtac | 53 |
| | ctttgtacgcctgtttgccgcccctcccccttttattaccacatttgtggtcgctgcaacgttagaag | |
| | attttaaagtagtgcaataggaagcatcacaaccagtggtgttatgcacaaacacttctgtttccc | |
| | cggagcgaggtatagacggtccccactgtcaaaagcctttaaccgttatccgccaaccaactac | |
| | gagacagctagtaatgccctgttctcctgcaaggcgttcgatcagacagatttcccctctgttagt | |
| | ctggtcgatgaggctagaagtaccccacaggtaactgtgttctagcctgcgtggcggccagcc | |
| | cagcttctgctgggacgcctttcaagagacatggtgtgaagactcaattgtgcttggttgtgagtc | |
| | ctccggcccctgaatgcggctaaccttaaacccggTAccatgctatgcaaaccagcatagtta | |
| | tggtcgtaatgggcaactctgggatgggaccgactactttgggtgtccgtgtttccatttttacttttt | |
| | gtgtcatttatggttacaattaagttgtaacc | |
| IHRV-B86 IRES | ttaaaacagcggatgggtaccctaccatccgacccactgggtgtagtacactggtacattgtacc | 54 |
| | tttgtacgcctgtttctccctattgtaccttccctcacaatcctaacccctgtaacgttagaagatgtg | |
| | caactcgtgcaataggagatactgcaaccagtggtatcaagcacaagaacttctgtttcctcgga | |
| | gcggagtatagatggtccccaccgtcaaaagctcttaaccgttatccgccaatcaactacgtga | |
| | cgactggtagcaTcattttgacactttgacgttcgatcaggtaggcttaccccacctactagtttg | |
| | gccgatgaggctgggagtccctcacaggtaactgtgtcccagcctgcgtggcggccaaccca | |
| | gcaattgctgggacgccaatttgttgacatggtgtgaagatcttaatgtgcttggttgtgagtcctc | |
| | cggcccctgaatgcggctaaccttaaccccggagccttgtgtcacaagccagtgacattaaggt | |
| | cgtaatgagtaatttcgggatgggaccgactactttgggtgtccgtgtttcctgtttttctttatgatt | |
| | gtcttatggtcacagcatatagtaattatatactgtgatc | |
| iHRV-B91 IRES | ttaaaacagcggatgggtttcccaccatccgacccactgggtgtagtactctggtacattgtacct | 55 |
| | ttgtacgcctgtttctcccctccctcccaacctatacaatcccggcaacttagaagatttgaactatt | |
| | gtacaataggcgctgtcacatccagtggcggctagtacaagcacttctgtttccccggagcgga | |
| | gtatagatggccaccgctgtcaaaagctcttaaccgttatccgccaattaactacgcaacggcta | |
| | gtaatatcatgtaacttttagggtgttcgatcaggtgagtgtacccctcactagtctggtcgatgag | |
| | gctgagaattccccacgggcgaccgtgtctcagcctgcgtggcggccagcccagcttatgctg | |
| | ggacgccttaattgtgacatggtgtgaagacccacgtgtgcttaattgtgagtcctccggcccct | |
| | gaatgcggctaacctaaaccctggagccttgaaacacaatccagtgttagcaaggtcgtaatga | |
| | gtaattccgggacgggaccgactactttgggtgtccgtgtttccttttatttttaagttgttcttatggt | |
| | cacaatataagtaatatatattgtgatc | |
| iHRV-B93 IRES | ttaaaacagtggatgggtttcctaccatccgacccactgggtgtagtgctctggtatattgtacctt | 56 |
| | tgcacgcctgttttcccattgtacccttccttaaattcctccccatgtaacgttagaagtttaagaac | |
| | ataaatgtacaataggaagcatcacatccagtggtgtatggtacaagcacttctgtttatccggag | |
| | cgaggtataagcggtacccactgctgaaagcctttaaccgtATaccgccaatcaactacgtaa | |
| | cggctagtattaccttgtttgcaatctggcgttcgatcaggtggttccccccactagtttggtcgat | |
| | gaggctaggagttccccacgggcgaccgtgtcctagcctgcgtggcggccaacccagcattt | |
| | gctgggacgcctttttgtagacatggtgtgaagacctgcatgtgcttgattgtgagtcctccggcc | |
| | cctgaatgcggctaaccttaaccccggagccttgtgacataatccaatgtcattaaggtcgtaat | |
| | gagcaattctgggacgggaccgactactttgggtgtccgtgtttctctttattctttacattgtcttat | |
| | ggtcacagcatatataatacatatatactgtgatc | |
| iHRV-B97 IRES | ttaaaacagcggatgggtttcccaccatccgacccactgggtgtagtgctctggtattttgtacctt | 57 |
| | tgcacgcctgtttcccctttgtacccatcctgaatttcctccctctgcaacgttagaagtttgtgaaa | |
| | ttaaaagtacaataggaagcatcacatccagtggtgttcagtacaagcactcctgtttccccgga | |
| | gcgaggtataggttgtacccacgaccgaaagcctttaaccgttatccgccaatcaactacataac | |
| | ggctagtatcatcatgtttttgatctggcgttcgatcaggtggtttcccccactagtctggtcgatga | |
| | ggctaggatttccccacgggcgaccgtgtcctagcctgcgtggcggccagcccagcttatgct | |
| | gggacgcctttttaaagacatggtgtgaagacctgcatgtgcttgattgtgagtcctccggcccct | |
| | gaatgcggctaaccttaaccctggagcccgacagcataatccaatgttgtttgggtcgtaatgag | |
| | caattccgggatgggaccgactactttgggtgtccgtgtttctttttattcttatattgtcttatggtca | |
| | cagcatatatagtatatatactgtgatc | |
| iHRV-B99 IRES | ttaaaacagcgggtgggtatcccaccacccgacccactgggtgtagtacactggtattttgtacc | 58 |
| | tttgtatgcctgtttgccatccctccctctcctcttacccttacccttattttgcggtaactttagaagtt | |
| | tgtaaatcacgcgcaatagggtataccacatccagtggtatgaagcacaagcaattcttgttccc | |
| | cggagctggatataaactgctaacgtggttgaaagtccttaaccgttatccgccagtTAactgc | |
| | gagaagactagtaactccatgtttgtttttaagcgttcgatcaggtggataccccattcactagtttg | |
| | gtcgatgaggctgggaatcccccacaggtaactgtgtctcagcctgcgtggcggccaaccca | |
| | gcttttgctgggacgcttactgacagacatggtgtgaagatcctattgcgcttaactgtgagtcct | |
| | ccggcccctgaatgcggctaacctcaaccccggagccttgtggtgtaaaccaacactcgcaag | |
| | gtcgtaacgagtaattctgggacgggaccgactactttgggtgtccgtgtttcctttatttcttattg | |
| | attgtctcctatggttacaaatttactgtaatc | |
| iHRVB_SC0739 | ttaaaacagcgggtgggtatcccaccacccgacccactgggtgtggtacactggtactttgtac | 59 |
| IRES | ctttgtatgcctgtttgcatccctttcccctcgcctaacatccttccccaaagtatattttgcggtaac | |
| | gttagaagaagtgaactcagtgcaacaggaagtatcaccaccagtgatataatgcacaagcaat | |
| | tcttgttccccggagctggatatagactgctaacgtggttgaaagtccttaaccgttatccgccag | |
| | ctaactgcgagaagactagtaacatcatgtacatttctagacgttcgatcaggtgaataccccatt | |
| | cactagtttggtcgatgaggctgagagttccccacaggtgactgtgtctcagcctgcgtggcgg | |
| | ccaacccgacttatgtcgggacgcctattaatggacatggtgcgaagatcctattgcgcttagct | |
| | gtgagtcctccggcccctgaatgcggctaaccttaaccccggagccttgtggtgcaaaccagc | |
| | atttgcaaggtcgtaatgagcaattctgggatgggaccgactactttgggtgtccgtgtttccttta | |
| | ttcttattatttgtgtttcatggttacaagcatagtttgtaatc | |
| iHRVB_G001-19 | TTAAAACAGCGGatgggtttcccaccatccgacccactgggtgtagtgctctggtatt | 60 |
| IRES | ttgtacccttgcacgcctgtttcccatttgtacccttccttaatttcctacccctgtaacattagaagtt | |
| | ttggaattttacggtacaataggaagcgccacatccagtggtgttgtgtacaagcacttctgtttac | |
| | ccggagcgaggtataggttgtacccacggccaaaaaccttcaaccgttatccgccaaccaact | |
| | acgtaacggctagtatcatcttgtatttgacttggcgttcgatcaggtggtatcccccactagtctg | |
| | gtcgatgaggctaggaattccccacgggcgaccgtgtcctagcctgcgtggcggccagccca | |
| | gctttagctgggacgccttttcaaagacatggtgtgaagacctgcatgtgcttggttgtgagtcct | |
| | ccggcccctgaatgcggctaaccttaaccccggagcccagtgacataatccaatgttatttgggt | |
| | cgtaatgagtaattccgggatgggaccgactactttgggtgtccgtgtttccttttattctttatattgt | |
| | cttatggtcacagcatatatagtatatatactgtgatc | |
| iHRVB_R93 | ttaaaacagcggatgggttctcccaccatccgacccactgggtgtagtactctggtattatgtacc | 61 |
| IRES | tttgtacgcctgtttattcccaaaccccctcccgttacatttccaacccctgtaacgttagaagtttg | |
| | gaacttaacagtacaataggaggcaccatatccaatggtgtaatgtacgagcacttctgtttcccc | |
| | ggagcgaggtataggtggtacccaccgctgaaagcctttaaccgttatccgccaatcaactacg | |
| | taacagctagtagcattttgttgttgactggacgttcgatcaggtggtttacccccccactagtttg | |
| | gtcgatgaggctggaaattccccacgggtgaccgtgttccagcctgcgtggcggccaaccca | |
| | gctcatgctgggacgccctttcaatgacatggtgtgaagactcgcatgtgcttgattgtgaatcct | |
| | ccggcccctgaatgcggctaaccctaaccccggagccttgcatcacaatccagtgatgttagg | |
| | gtcgtaatgagtaattctgggatgggaccgactactttgggtgtccgtgtttctcatttttctttgaat | |
| | tgtcttatggtcacagcatatagtaatatatactgtgatc | |
| iHRVB_BCH214 | ttaaaacagcgggtgggtatcccaccacccgacccactgggtgtggtacactggtactttgtac | 62 |
| IRES | ctttgtatgcctgtttgcatcccttttcccttcacctaacatcctcccccaaagtatattttgcggtaac | |
| | gttagaagaagtgaactcagtgcaacaggaagtatcaccaccagtgatataacgcacaagcaa | |
| | ttcttgttccccggagctggatatagactgctaacgtggtcaaaagtccttaaccgttatccgcca | |
| | gctaactgcgagaagactagtaacatcatgcacatttctagacgttcgatcaggtgaatacccca | |
| | ttcactagtttggtcgatgaggctgagagttccccacaggtgactgtgtctcagcctgcgtggcg | |
| | gccaacccgacttatgtcgggacgcctattaatggacatggtgtgaagatcctattgcgcttagc | |
| | tgtgagtcctccggcccctgaatgcggctaaccttaaccccggagccttgtggtgcaaaccagc | |
| | atttgcaaggtcgtaatgagcaattctgggatgggaccgactactttgggtgtccgtgtttccttta | |
| | ttcttattatttgtgtttcatggttacaagcatagtttgtaatc | |
| iHRVB_3039 | ttaaaacagcggatgggtatcccaccatccgacccactgggtgtagtactctggtattatgtacct | 63 |
| IRES | ttgtacgcctgtttctccctccctaccaccccgccccacgtatcccgtaacttagaagaattgaac | |
| | aatcgcacaataggagctattacaaccagtaatagttggtgcaagcacttctgtttccccggagc | |
| | ggagtatagacggccaccgctgtcaaaagctcttaaccgttacccgccagCtaactacgtaac | |
| | ggctagtaacatcttgtcgtgcttagggtgttcgatcaggtgaagaaaaacccccttcactagtct | |
| | ggtcgatgaggctgagaagtccccacgggtaaccgtgtctcagcctgcgtggcggccagccc | |
| | agcatatgctgggacgccttaagtatgacatggtgtgaagacccgcatgtgcttagctgtgagtc | |
| | ctccggcccctgaatgcggctaacctaaaccctggagccttggagcacaagccagtgcttgca | |
| | aggtcgtaatgagcaattccgggacgggaccgactactttgggtgtccgtgtttccttttatctcat | |
| | tattgttcttatggttacaatatatagagtatattgtaatc | |
| iEV-B69 IRES | ttaaaacagcctgtgggttgtacccacccacagggcccactgggcgctagcacactggtatca | 64 |
| | cggtacctttgtgcgcctgttttataccccctaccccaactttaaacttagaagcaaagcaaaccc | |
| | gatcaatagcagatgtagcaagccagttgcatcttgatcaagtacttctgtctccccggactgagt | |
| | atcaatagactgctcacgcggttgaaggagaaaacgttcgttacccgaccaactacttcgagaa | |
| | acctagtaacaccatgaaagtagcagggtgtttcgatcagcacaaccccagtgtagatcaggct | |
| | gatgagtcaccgcatcccccacgggcgaccgtggcggtggctgcgctggcggcctgcccat | |
| | ggggcaacccatgggacgctctaatatggacatggtgtgaagagtctattgagctagttggtag | |
| | tcctccggcccctgaatgcggctaatcctaactgcggagcacataccccaaagccaaggggc | |
| | agtgtgtcgtaacgggcaactctgcagcggaaccgactactttgggtgtccgtgtttccttttattc | |
| | tcaaactggctgcttatggtgacaatctcagagttgttaccatatagctattggattggccatccgg | |
| | tgagcaacagagcaattgtctaccaatttgttggatttattccactcaatctagcggttttcagaaca | |
| | ttgaattatattctaaccctcaacaagacgaa | |
| iEV-B73 IRES | ttaaaacagcctgtgggttgtacccacccacagggcccactgggcgctagcacactggtatca | 65 |
| | cggtacctttgtgcgcctgttttataccccctaccccaactttaaacttagaagcaaagcaaaccc | |
| | gatcaatagcagatgtagcaagccagttgcatcttgatcaagtacttctgtctccccggactgagt | |
| | atcaatagactgctcacgcggttgaaggagaaaacgttcgttacccgaccaactacttcgagaa | |
| | acctagtaacaccatgaaagtagcagggtgtttcgatcagcacaaccccagtgtagatcaggct | |
| | gatgagtcaccgcatcccccacgggcgaccgtggcggtggctgcgctggcggcctgcccat | |
| | ggggcaacccatgggacgctctaatatggacatggtgtgaagagtctattgagctagttggtag | |
| | tcctccggcccctgaatgcggctaatcctaactgcggagcacataccccaaagccaaggggc | |
| | agtgtgtcgtaacgggcaactctgcagcggaaccgactactttgggtgtccgtgtttccttttattc | |
| | tcaaactggctgcttatggtgacaatctcagagttgttaccatatagctattggattggccatccgg | |
| | tgagcaacagagcaattgtctaccaatttgttggatttattccactcaatctagcggttttcagaaca | |
| | ttgaattatattctaaccctcaacaagacgaa | |
| iEV-B74 IRES | ttaaaacagcctgtgggttgttcccactcacagggcccactgggcgctagcacactggtatcac | 66 |
| | ggtacccttgtgcgcctgttttattcaccccccccaaatgtaacttagaagcataacccaaatggt | |
| | caatagacagcccagtacaccagctgggtttcggccaagtacttctgttaccccggaccgagta | |
| | tcaataggctgctcacgcggctgaaggagaaaacgttcgttatccggccaattacttcgagaaa | |
| | cctagtaacaccatgaaagttgcgcagtgtttcgctccgcacaaccccagtgtagatcaggtcg | |
| | atgagtcaccgcgttccccacgggcgaccgtggcggtggctgcgttggcggcctgcccatgg | |
| | ggcaacccatgggacgcttcaatactgacatggtgcgaagagtctattgagctaattggtagtcc | |
| | tccggcccctgaatgcggctaatcctaactgcggagcagatacccacatgccagtgggcggtc | |
| | tgtcgtaacgggcaactccgcagcggaaccgactactttgggtgtccgtgtttccttttattcttaa | |
| | tctggctgcttatggtgacaattgaaagattgttgccatatagctattggattggccatccggtgac | |
| | aaacagggcgattatttatttgtttgttggttttgttccattaaactatagagttttgaatactctagaat | |
| | ttattataacactcaacacaacaaa | |
| iEV-B75 IRES | tttaaaacagtcctgtgggttgttacccacccacagggcccactgggcgccagcacactggtat | 67 |
| | cacggtacctttgtgcgcctgttttatatacccttcccctactaagtaacttagaagcaattaaacaa | |
| | agaccaacagtaagcacagcgcaccagctgtgtttcggtcaagcacttctgttaccccggactg | |
| | agtatcaataggctactcacgtggctgaaggagaaaacgttcgttacccgaccaattacttcgag | |
| | aagcctagtaacaccatggaggttgcgcggcgtttcgctccacacaaccccagtgcagatcag | |
| | gtcgatgagccaccgcattccccacgggcgaccgtggcggtggctgcgttggcggcctgccc | |
| | atggggcaacccatgggacgcttcaatactgacatggtgcgaagagtctattgagctaattggt | |
| | agtcctccggcccctgaatgcggctaatcccaactgcggagcagatacccacgcgccagtgg | |
| | gcagtctgtcgtaatgggcaactctgcagcggaaccgactactttgggtgtccgtgtttcctttatt | |
| | cttatactggctgcttatggtgacaattgagagattgttaccatatagctattggattggccatccg | |
| | gtgacgagcagagctattatttacctatttgttggttttataccattgaactataaagttttaaaaacct | |
| | taaacttcatcttagtactcaatacagcaaa | |
| iEV-B77 IRES | ttaaaacagcctgtgggttgtacccacccacagggcccattgggcgctagcacactggtattcc | 68 |
| | ggtacccttgtgcgcctgttttatctacccttccctactgcaacttagaagttaaacagtcacggtc | |
| | gataggcggttcagtaaaccaactggatcttgaccaagcacttctgttaccccggactgagtatc | |
| | aataagctgcttacgtggctgaaggagaaaacgttcgttacccggccaattacttcgagaaacct | |
| | agtaccaccatgaaggttgcgcagcgtttcgctccgcacaaccccagtgtagatcaggtcgatg | |
| | agtcaccgcactcctcacgggcgaccgtggcggtggctgcgctggcggcctgcccatgggg | |
| | caacccatgggacgcttcaatactgacatggtgcgaagagtctattgagctaattggtaatcctc | |
| | cggcccctgaatgcggctaatcccaactgcggagcaggtacccacacaccagtgggcagcct | |
| | gtcgtaacgggcaactctgcagcggaaccgactactttgggtgtccgtgtttccttttattcctata | |
| | ctggctgcttatggtgacaattgaaagattgttaccatatagctattggattggccatccagtgaca | |
| | aacagagcaattgtctatctatttgtcggttttgtaccaataaatttcaaagtcctaaaaaccctcaa | |
| | tttcatattgttacttaacacaacaag | |
| iEV-B79 IRES | ttaaaacagcctgtgggttgtacccacccacagggcccattgggcgctagcacactggtatcac | 69 |
| | ggtacccttgtgcgcctgttttatccgcccctccccattgtaacttagaagataactaaacacggt | |
| | cgttaggtggctcagtacgccaactgagtcttgaccaagcacttctgttaccccggactgagtat | |
| | caataggctgctcacgcggctgaaggagaaaacgttcgttatccggccaattacttcgagaaac | |
| | ctagtaccaccatggaggttgcgcagtgtttcgctccgcacaaccccagtgtagatcaggccga | |
| | tgagtcaccgcattccccacgggcgaccgtggcggtggctgcgctggcggcccgcccatgg | |
| | gtaacccatgggacgcttcaatactgacatggtgtgaagagtctattgagctaattggtagtcctc | |
| | cggcccctgaatgcggctaatcccaactgcggagcagatacccacacaccagtgggcagtct | |
| | gtcgtaacgggcaactctgcagcggaaccgactactttgggtgtccgtgtttccttttattccaata | |
| | ctggctgcttatggtgacaattgaaagattgttaccatatagctattggattggccatccggtgaca | |
| | aacagagcgattgtttacctatttgtaggtttcataccattgaattttaaagttttaagaacccttaact | |
| | ttatattagcacttaacacaacaaa | |
| iEV-B80 IRES | ttaaaacagcctgtgggttgatcccacccacagggcccactgggcgtcagcactctggtatcac | 70 |
| | ggtacctttgtgcgcctgttttatactccctccccccaaccgtaacttagaagcagcactaactgat | |
| | caatagccagcgcggcatgccagccacgcttcgatcaagcacttctgttaccccggactgagt | |
| | atcaatagactgctcacgcggttgaaggagaaaacgttcgtcatccggccaactacttcgagaa | |
| | acctagtaacaccatggaagttgcggagtgtttcgctcagcactaccccagtgtagatcaggtc | |
| | gatgagtcaccgcattccccacaggcgactgtggcggtggctgcgttggcggcctgcccatg | |
| | gggaaacccatgggacgctcttatatagacatggtgcgaagagtctattgagctagttggtagtc | |
| | ctccggcccctgaatgcggctaatcctaactgcggagcatacaccctcaaaccagagggcagt | |
| | gtgtcgtaatgggcaactctgcagcggaaccgactactttgggtgtccgtgtttcactttatttcta | |
| | aattggctgcttatggtgacaattgagagattgttaccatatagctattggattggccatccggtga | |
| | ccaatagagctattatatatctctttgttgggttcataccacttagcttgaaagaggttaaaactctg | |
| | cattacattatactattgaattccgcaaa | |
| IEV-B81 IRES | ttaaaacagcctgtgggttgtacccacccacagggcccattgggcgcttgcacactggtattcc | 71 |
| | ggtacctttgtgcgcctgttttatacatcctacccccgtaacttagaagcaagatcacccgatcac | |
| | caggaaatgtggcaaaccagtcacattctgatcaagcacttctgtttccccggactgagtatcaat | |
| | aggctgttcacacggctgaaggagaaaacgttcgttacccggccaactacttcgaaaaaccta | |
| | gtatcaccgtgaacgttgcagagtgtttcgctcagcactttccccgtgtagatcaggtcgatgagt | |
| | caccgcattccccacgggcgaccgtggcggtggctgcgttggcggcctgcctgcggggcaa | |
| | cccgtaggacgctctaatacagacacggtgtgaagagtctattgagctagttggtagtcctccg | |
| | gcccctgaatgcggctaatcctaactgcggagcgagcgcttgtaatccaacaggtagctcgtc | |
| | gtaacgggtaactctgcagcggaaccgactactttgggtgaccgtgtttccttttatttcattactg | |
| | gctgcttatggtgacaatcacagagttgttaccatatagctattggattggccatccggtgtcaaat | |
| | agagcaattgtgtttctttttgttgggtttgtaccacttgacttgaccagctacaacactctgtgctac | |
| | attgttagattgaacacagcaaa | |
| iEV-B82 IRES | ttaaaacagcctgtgggttgcacccacccgcagggcccactgggcgctagtacactggtaccc | 72 |
| | cggtacccttgtacgcctgttttatttctccctcccccaaccagtaacctagaagtaaagcaaacc | |
| | cgatcaatagcaggtgcgacacaccagtcgtatcttgatcaagcacttctgtaaccccgggccg | |
| | agtatcaatagactgctcacgcggttgagggagaaaacgctcgttatccggctgactacttcga | |
| | gaaacccagtaacaccatagaagttgcagagtgtttcgctcagcactttccccgtgtagatcagg | |
| | tcgatgagtcaccgcattccccacgggtgaccgtggcggtggctgcgttggcggcctgcctat | |
| | ggggcaacccatgggacgctctaatacagacatggtgtgaagagtcaattgagctagtcagtat | |
| | tcctccggcccctgaatgcggctaatcctaactgcggagcacgtgcctccaatccagggggtg | |
| | gcgtgtcgtaacgggtaactctgcagcggaaccgactactttgggtgtccgtgtttccttttattctt | |
| | atcttggctgcttatggtgacaattgaaggattgttaccatatagctattggattggccatccggtg | |
| | actaacagagcaatcatataccaatttgttggttttataccattgaattacaaagttctaatcaccctt | |
| | aaattcattttaacacttaatacgtcaaa | |
| iEV-B83 IRES | tttaaaacagcctgtgggttgttcccacccacagggcccactgggcgctagcactctggtatca | 73 |
| | cggtacccttgtgcgcctgttttatatccccaccccagtggaacattagaagcagcacacctatg | |
| | atcaatagtgggcgtggcatccagtcacgttatgatcaagcacttctgtgaccccggactgagta | |
| | tcaatagactgctcacgcggttgaaggagaaaacgttcgttacccggccagttacttcgagaaa | |
| | cccagtaacaccatgaatgttgcagagtgtttcgctcagcacttcccccgtgtagatcaggtcga | |
| | tgagccactgcattccccacgggcgaccgtggcagtggctgcgttggcggcctgcctatggg | |
| | gtcacccataggacgctctaatactgacatggtgcgaagagtctattgagctaactggtagtcct | |
| | ccggcccctgaatgcggctaatcctaactgtggagcacattcccccaatccagggggcagtgt | |
| | gtcgtaacgggcaactctgcagcggaaccgactactttgggtgtccgtgtttctttttattctcatat | |
| | tggctgcttatggtgacaattacagaattgttaccatatagctattggattggccatccggtgacta | |
| | acagagcaattatttatcaattcgttggatttgtcccactcgatttcaaagcagtcaatacactctca | |
| | tatatcgttacactcaacacaacaaa | |
| iEV-B84 IRES | ttaaaacagcctgtgggttgttcccacccacagggcccaccggggccagcacactggtatca | 74 |
| | cggtacccttgtgcgcctgttttatccaccccttcccacagtaacttagaagcacaccatgtatac | |
| | ggtcaataggcggctcagtacaccaactgggccacgaccaagcacttctgttaccccggactg | |
| | agtatcaataagctgctcacgcggctgaaggagaaaacgttcgttacccggccaattacttcga | |
| | gaaacccagtaccaccatgaaggttgcgcagcgtttcgttccgcacaaccccagtgtagatca | |
| | ggtcgatgagtcaccgcgttcctcacgggtgaccgtggcggtggctgcgttggcggcctgccc | |
| | atggggcaacccatgggacgcttcaatactgacatggtgcgaagagtctattgagctaattggt | |
| | agtcctccggcccctgaatgcggctaatcctaactgcggagcagatacccacaaaccagtggg | |
| | cagtctgtcgtaacgggcaactctgcagcggaaccgactactttgggtgtccgtgtttccttttatc | |
| | cttacactggctgcttatggtgacaattgagaaattgttaccatatagctattggattggccatccg | |
| | gtgacaaacagagcagttgtttacctatttgttggtttcgtaccgctgaaccttaaagttctaaaga | |
| | ccctcaattttatcttagcacttaacacagcaaa | |
| iEV-B85 IRES | ttaaaacagcctgtgggttgttcccacccacagggcccaccgggcgctagcacactggtattgc | 75 |
| | ggtacctttgtgcgcctgttttatacacccctccttaatgtaactcagaagtacaacattaccggtc | |
| | aacagatgactcagtaaaccaactgagcctcgaccaagcacttctgtttccccggaccgagtat | |
| | caataggctgctcacgcggctgaaggagaaagtgttcgttacccggccaattacttcgagaag | |
| | cccagtaccaccatggaggttgcgcggcgtttcgctccgcacatccccagtgtagatcaggct | |
| | gatgagtcaccgcgttccccacgggtgaccgtggcggtggctgcgctggcggcctgcccatg | |
| | gggtaacccatgggacgcttcaatactgacatggtgtgaagagcctattgagctaattggtagtc | |
| | ctccggcccctgaatgcggctaatcctaactgcggagcagatacccacacaccagcgggcag | |
| | tctgtcgtaacgggcaactctgcagcggaaccgactactttgggtgtccgtgtttccttttatttttat | |
| | gctggctgcttatggtgacaattgaggaattgttaccatatagctattggattggccatccagtgtc | |
| | caacagagcgatcatttacctgttcgttgggttcgtaccactaaaccacaagtcactcagcacac | |
| | ttaattacattataactctcaacacaacaag | |
| iEV-B86 IRES | ttaaaacagcctgtgggttgtacccacccacagggcccactgggcgctagcacactggtattac | 76 |
| | ggtacccttgtgcgcctgttttatttacccctcccccacgcaacctagaagcatgacttaagcggt | |
| | caatagatggctcagtatgccaactgagttatgaccaagcacttctgttaccccggaccgagtat | |
| | caataggctgctcacgcggctgaaggagaaagtgttcgttacccggccagttacttcgagaaa | |
| | cctagtatcatcatgaaggttgcgaggcgtttcgctccacacgaccccggtgtagatcaggctg | |
| | atgagtcaccgcattccccacgggcgaccgtggcggtggctgcgctggcggcctgcccatgg | |
| | ggcaacccatgggacgcttcaatattgacatggtgcgaagagtctattgagctaactggtagtcc | |
| | tccggcccctgaatgcggctaatcctaactgcggagcagacacccacatgccagtgggcagt | |
| | ctgtcgtaacgggcaactctgcagcggaaccgactactttgggtgtccgtgtttccttttattcttat | |
| | attggctgcttatggtgacaattgaaagattgttgccatatagctattggattggccatccagtgac | |
| | taacagagcaattgtctacctgttcgttggttacgtaccactaaattataaagttttgaacacactta | |
| | attttattatagcacttaacaccttaaa | |
| iEV-B87 IRES | ttaaaacagcctgtgggttgttcccacccacagggcccatagggcgccagcactctggtatccc | 77 |
| | ggtacctttgtgcgcctgttttatccacccttccccaagcgtaacttagaagcccatcatacacgg | |
| | tcaataggtggttcagtatgccaactgagtcttgaccaagcacttctgttaccccggactgagtat | |
| | caataagctgctcacgcggctgaaggagaaaacgttcgttatccggccaattacttcgagaaac | |
| | ctagtaacaccatgaaggttgcgcagcgtttcgctccgcacaaccccagtgtagatcaggtcga | |
| | tgagtcaccgcactccccacgggcgaccgtggcggtggctgcgttggcggcctgcccatggg | |
| | gcaacccatgggacgcttcaatactgacatggtgcgaagagtctattgagataattggtagtcct | |
| | ccggcccctgaatgcggctaatcctaactgcggagcagatacccacatgccagtgggcagtct | |
| | gtcgtaacgggcaactctgcagcggaaccgactactttgggtgtccgtgtttccttttatttctata | |
| | ctggctgcttatggtgacaattgacagattgttaccatatagctattggattggccatccggtgac | |
| | gaacagagcaattatttacttatttgttggttttataccattaaatttcaaagttttaaaaaccctcaac | |
| | ttagttttgtcactcaactcagcaaa | |
| iEV-B88 IRES | ttaaaacagcctgtgggttgatcccacccacagggcccactgggcgctagcacactggtattcc | 78 |
| | ggtacttttgtgcgcctgttttatacacccttccccttcatgtaacttagaagtaaaacacaaacggt | |
| | caataggtagctcagcaaaccagctgagcctcggccaagcacttctgttaccccggactgagt | |
| | atcaataagctgttaacacggctgaaggagaaaacgttcgttatccggccaattacttcgagaaa | |
| | cctagtaacaccatgaaggttgcgtagcgtttcgctccacacaaccccagtgtagatcaggtcg | |
| | atgagtcaccgctctccccacgggtgaccgtggcggtggctgcgttggcggcctgcccatgg | |
| | agcactccatgggacgcttcaatactgacatggtgtgaagagtctattgagctaattggtagtcct | |
| | ccggcccctgaatgcggctaatcctaactgcggagcagacacccacacgccagtgggcagtc | |
| | tgtcgtaatgggcaactctgcagcggaaccgactactttgggtgtccgtgtttccttttattcttaca | |
| | ttggctgcttatggtgacaatcaaggaattgttaccatatagctattggattggccatccggtatct | |
| | aacagagcaattgtttacctcttcaccggttttgtaccattgaattgcaatagtctcaacaccctttg | |
| | ctatatcattaaattgaactcagcaaa | |
| iEV-B93 IRES | aaagcaggctttaaaacagctctggggttgttcccacccacagggcccactgggcgctagcac | 79 |
| | tctggtatcacggtacctttgtgcgcctgtttttccacccctccccatacgcaacttagaagagag | |
| | actcaaccggtcaatagacagcctagtacaccaactaggttctgaccaagaacttctgtttcccc | |
| | ggaccgagtatcaataggctgcttacgcggctgaaggagaaaacgttcgttacccggccaatt | |
| | acttcgagaaacctagtatcaccatggaagttgcgcagtgtttcgctccgcacaaccccagtgta | |
| | gatcaggtcgatgagtcaccgcattccccacaggcgactgtggcggtggctgcgttggcggc | |
| | ctgcccgtggggcaacccacgggacgcttcaataccgacatggtgcgaagagtctattgagct | |
| | aattggtagtcctccggcccctgaatgcggctaatcctaactgtggagcagatacccacgcgcc | |
| | agtgggcagtctgtcgtaatgggcaactccgcagcggaaccgactactttgggtgtccgtgtttc | |
| | cttttatcctcataatggctgcttatggtgacaattgaaagattgttaccatatagctattggattggc | |
| | catccggtgactaacagagcaattatttatttgtttgttgggtttgtaccattgaactacaaggtcctt | |
| | aatactctaaactacattataagtcttaatacagtgca | |
| iEV-B97 IRES | ttaaaacagcctgtgggttgttcccacccacagggcccactgggcgccagcacactggtatca | 80 |
| | cggtacctttgtgcgcctgttttatccacccttccccaatgtaacttagaagtaaaactagaaagat | |
| | caatagcaagcacagtatgccaactgtgttatgatcaagcacttctgttaccccggactgagtatc | |
| | aataagctgctaaagcggctgaaggagaaaacgttcgttacccggccaactacttcgagaaac | |
| | ccagtaccaccatgaaggttgcgcagtgtttcgctccacacaatcccagtgtagatcaggtcgat | |
| | gagtcaccgcgttccccacgggcgaccgtggcggtggctgcgttggcggcctgcccatggg | |
| | atgacccatgggacgcttcaatactgacatggtgcgaagagtctattgagctagttggtagtcct | |
| | ccggcccctgaatgcggctaatcctaactgcggagcagatacccacacaccagtgggcagtct | |
| | gtcgtaacgggtaactctgcagcggaaccgactactttgggtgtccgtgtttctttttatcctcaaa | |
| | ttggctgcttatggtgacaattgataagttgttaccatatagctattggattggccatccggtgtcta | |
| | acagagctattgtttatttatttgttggtttcgtgccactaaattacaaagtttttgaaactctcaacttc | |
| | attttgacactcaataaagcaaa | |
| iEV-B98 IRES | ttaaaacagcctgtgggttgttcccacccacagggcccaatgggcgccagcactctggtattac | 81 |
| | ggtacctttgtgcgcctgttttatatcccctcccccaattgtaacttagaagcaacacgcaccgac | |
| | caacagtgggcgtggcacaccagccacgtcttggtcaagcacatctgtttccccggactgagta | |
| | tcaatagactgctcacgcggtcgaaggagaaaacgttcgttatccggccaactacttcgagaag | |
| | cccagtaacaccatgaatattgcgaagtgtttcgctcagcacactcccagtgtagatcaggtcga | |
| | tgagtcaccgcattccccacgggcgaccgtggcggtggctgcgttggcggcctgcctatggg | |
| | gcaacccgtaggacgcttcaatatggacatggtgtgaagagtctattgagctagttggtagtcct | |
| | ccggcccctgaatgcggctaatcctaaccgcggagcaggcacccacaaaccagtgggcagc | |
| | ttgtcgtaacgggtaactctgcggcggaaccgactactttgggtgtccgtgtttccttttattcttat | |
| | actggctgcttatggtgacaattgagagattgttgccatatagctattggattggccatccggtga | |
| | caaacagagcaattgtttatctatttgttggttttataccgttaagttttaaagtcctaaaaacccttaa | |
| | cttggtcatactactcaactcagcaaa | |
| iEV-B100 IRES | ttaaaacagtctgtgggttgttcccacccacagggcccacgtggcgccagcacactggtatctc | 82 |
| | ggtacctttgtgcgcctgttttatccaccctaaaccccgttgtaacttagaagatgagcaacactg | |
| | atcaatagtgggtgcaacatgccagttgcatcacgatcaagcacttctgtatccccggactgagt | |
| | atcaataggctgctcacgcggctgaaggagaaaacgttcgttatccggccagttacttcgagaa | |
| | acctagtaacatcatggaggttgcgcagtgtttcgctccgcacacccccagtgtagatcaggtc | |
| | gatgagtcaccgcgttccccacgggcgaccgtggcggtggctgcgttggcggcctgcccatg | |
| | ggataacccatgggacgcttcaatactgacatggtgtgaagagtctattgagctaactggtagtc | |
| | ctccggcccctgaatgcggctaatcctaactgtggagcagacacccacatgccagtgggcagt | |
| | ctgtcgtaacgggcaactccgcagcggaaccgactactttgggtgtccgtgtttccttttattccta | |
| | tattggctgcttatggtgacaattgaaagattgttaccatatagctattggattggccatccggtgtc | |
| | aaacagagcaatcatttatttgtttgttggttttatacctctgaactttaaagtgctaaagactctcaat | |
| | tttatattatatctcaacaaatcagg | |
| iEV-B101 IRES | ttaaaacagcctgtgggttgttcccacccacagggcccactgggcgccagcactctggtattac | 83 |
| | ggtacctttgtgcgcctgttttatatccccctccccacttgaaacttagaagtgaaacacactgatc | |
| | aatagttgacatggcaagccagccatgtcttgatcaagcacctctgtttccccggacctagtatca | |
| | atagactgctcgcgcggttgaaagagaaagcgttcgttacccggctaactacttcgagaaactt | |
| | agtaacaccatggaagttgcggagtgtttcgctcagcacatccccagtgtagatcaggccgatg | |
| | agtcaccgcattccccacgggcgaccgtggcggtggctgcgttggcggcctgcctgtggggc | |
| | aacccacaggacgctctaatacaaacatggtgcgaagagtctattgagctagttagtagtcctcc | |
| | ggcccctgaatgcggctaatcctaactgcggagcacgtgcttccaatccaggaggtggcgtgt | |
| | cgtaacgggtaactctgcagcggaaccgactactttgggtgtccgtgtttccttttattcttgcattg | |
| | gctgcttatggtgacaattgagagattgttaccatatagctattggattggccatccggtgacaaa | |
| | cagagcaattatatatctctttgttggatacattccattgaactacaaggagttaaaaaccctcaatt | |
| | atattctgttcttgaattccgcaag | |
| iEV-B106 IRES | ttaaaacagcctgtgggttgttcccacccacagggcccatggggcgccagcacactggtattg | 84 |
| | cggtacctttgtgcgcctgttttattcaccctttcccaatgaaacttagaagcttaactcaaccggtc | |
| | gatagacagcccagtacaccaactgggttttgaccaagtactcctgtttccccggactgagtatc | |
| | aataagctgctcgcgcggctgaaggagaaaacgttcgttacccggccaattacttcgagaaac | |
| | ctagtaccaccatgaacgttgcgcagtgtttcgctccgcacaaccccagtgtagatcaggtcgat | |
| | gagtcaccgcaatccccacgggcgaccgtggcggtggctgcgctggcggcctgcccatggg | |
| | gcatcccatgggacgcttcaatactgacatggtgcgaagagtctattgagctaattggtaatcctc | |
| | cggcccctgaatgcggctaatcctaactgcggggcagacacccacacgccagtgggcagtct | |
| | gtcgtaacgggtaactccgcagcggaaccgactactttgggtgtccgtgtttccttttatctttatat | |
| | tggctgcttatggtgacaattgaaagattgttgccatatagctattggattggccatccggtgtcta | |
| | atagagcaattatatatctctttgttggcttcatcccgttaaataaagtgagctaccagaccctgtgc | |
| | tatattatcaaattgaacacaataaa | |
| iEV-B107_ | ttaaaacagcctgtgggttgttcccacccgcagggcccactgggcgctagcacactggtatccc | 85 |
| TN94-0349 | ggtacccttgtgcgcctgttttatataccctcccccttatgtaacttagaagtatgattcaaacggtc | |
| IRES | gacaggcggctcagtgcaccaactgagtcatgaccaagcacttctgttaccccggactgagtat | |
| | caataagctgttcacacggctgaaggagaaaacgttcgttacccggccaattacttcgagaaac | |
| | ctagtaccaccatgaaggttgcgcagtgtttcgctccacacaaccccagtgtagatcaggtcgat | |
| | gagtcaccgcattccccacgggcgaccgtggcggtggctgcgttggcggcctgcccatggg | |
| | gcaacccatgggacgcttcaatactgacatggtgtgaagagtctattgagctaattggtagtcct | |
| | ccggcccctgaatgcggctaatcccaactgtggagcagatactcacaaaccagtgagcggtct | |
| | gtcgtaacgggcaactccgcagcggaaccgactactttgggtgtccgtgtttctttttattcttaca | |
| | ttggctgcttatggtgacaattgacaaattgttaccatatagctattggattggccatccggtgaca | |
| | aacagagctattgtttacttgtttgttggtttcataccattaaattacaaggtcttagaaactctcaact | |
| | ttattttgacactcaatacagcaaa | |
| IEV-B111 IRES | ttaaaacagcctgtgggttgttcccacccacagggcccactgggcgccagcacactggtatca | 86 |
| | cggtacccttgtgcgcctgttttataaacccttccccaatgcaacttagaagctcaactttaccggt | |
| | caatagacagtccagtacgccaactgggcttcgatcaagcacttctgttaccccggactaagtat | |
| | caataggctgctcacgcggctgaaggagaaaacgttcgttacccggccaattacttcgagaaa | |
| | cccagtaacactatggaggttgcgcagtgtttcgttccacacaaccccagtgtagatcaggtcg | |
| | atgagtcaccgctttccccacgggcgaccgtggcggtggctgcgttggcggcctgcccatgg | |
| | ggcaacccatgggacgcttcaatactgacatggtgtgaagagtctattgagctaattggtattcct | |
| | ccggcccctgaatgcggctaatcctaactgcggagcagatactcacgagccagtgggcagtct | |
| | gtcgtaacgggtaactctgcagcggaaccgactactttgggtgtccgtgtttccttttattcatatat | |
| | tggctgcttatggtgacaattgagagattgttaccatatagctattggattggccatccggtgacta | |
| | atagagcaattgtttacctatttgttggctttgtgccgttgaactacaaggttttaaaaactattaact | |
| | acatcctagtacttaacacgttgaa | |
| iEV-B113 IRES | actctggtattgcggtacctttgtacgcctgttttatatcccttccccatgtaactttagaagcaattc | 87 |
| | acaaggttcaatagagggggtacaaaccagtaccaccacgaacaagcacttctgtttccccggt | |
| | gaagctgtatagactgtccccacggtcaaaaacggcagatccgttatccgcttatgtacttcgag | |
| | aagcctagtatcatcttggaatcttcgatgcgttgcgttcagcactctatcccgagtgtagcttagg | |
| | tcgatgagtctgggcattccccaccggcgacggtggcccaggctgcgttggcggcctacccat | |
| | ggctaacgccatgggacgctagttgtgaacaaggtgtgaagagcctattgagctacatgagag | |
| | tcctccggcccctgaatgcggctaatcccaaccacggagcaagtactcacaacccagtgggc | |
| | agcttgtcgtaacgcgtaagtctgtggcggaaccgactactttgggtgtccgtgtttcctttttatttt | |
| | tgatatggctgcttatggtgacatctaagattgttatcatatagcttttggattggccatccggtgatt | |
| | tttgagactaaaacgcttgtttgccttatcacacaatttagtattttgttatattcatacggtcata | |
| iEV-D68 IRES | ttaaaacagctctggggttgttcccactcaagggcccacgtggcggctagtactctggtatctcg | 88 |
| | gtacctttgtacgcctgttttaattccctccccaacgtaacttagaagcttttaaaccaaagctcaat | |
| | aggtggagcgcaaaccagcgctcttatgagcaagcacttctgtctccccggtgtggttgtatag | |
| | actgtccccacggttgaaaacaacttatccgttaCccgctatagtacttcgagaaacctagtattg | |
| | ccttcggagtgttgatgcgttgcgctcagcacactaacccgtgtgtagcttgggtcgatgagtct | |
| | ggacgtaccccactggcgacagtggtccaggctgcgttggcggcctactcatggtgaaaacc | |
| | atgGgacgctagacatgaacaaggtgtgaagagtctattgagctgctatagagtcctccggcc | |
| | cctgaatgcggctaatcctaaccatggagcaagtgctcacaaaccagtgagttacttgtcgtaac | |
| | gcgcaagtccgtggcggaaccgactactttgggtgtccgtgtttcactttttacttttatgactgcta | |
| | atggtgacaatttaatattgttaccatttggcttgtcgaattgatcacataagatctatagttttgttca | |
| | ctgatttgctttgaaataatctcacctcaaaacctccagtacataacatttaaagagtttaaacttatt | |
| | tataaca | |
| iEV-D70 IRES | ttaaaacagctctggggttgttcccaccccagaggcccacgtggcggctagtactccggtaccc | 89 |
| | cggtacccttgtacgcctgttttatactccctttcccaagtaactttagaagaaataaactaatgttc | |
| | aacaggagggggtacaaaccagtaccaccacgaacacacacttctgtttccccggtgaagttg | |
| | catagactgtacccacggttgaaagcgatgaatccgttacccgcttaggtacttcgagaagccta | |
| | gtatcatcttggaatcttcgatgcgttgcgatcagcactctaccccgagtgtagcttgggtcgatg | |
| | agtctggacaccccacaccggcgacgtggtccaggctgcgttggcggcctacccatggctag | |
| | caccatgggacgctagttgtgaacaaggtgcgaagagcctattgagctacctgagagtcctcc | |
| | ggcccctgaatgcggctaatcccaaccacggagcaaatgctcacaatccagtgagtggtttgtc | |
| | gtaatgcgcaagtctgtggcggaaccgactactttgggtgtccgtgtttccttttatttttattatggc | |
| | tgcttatggtgacaatctgagattgttatcatatagctattggattagccatccggtgaCatcttga | |
| | aattttgccataactttttcacaaatcctacaacattacactacactttctcttgaataattgagacaa | |
| | ctcata | |
| iEV-D94 IRES | ttaaaacagcctgtgggttgttcccaccccagaggcccacgtggcggccagtactctggtatca | 90 |
| | cggtacctttgtacgcctgttttatatccccttcccccgcaacttagaagaaaacaaatcaagttca | |
| | ctaggagggggtacaaaccagtaccaccacgaacaagcacttctgtttccccggtgatgtcgta | |
| | tagactgtaaccacggttgaaaacgattgatccgttatccgctcttgtacttcgaaaagcccagta | |
| | tcaccttggaatcttcgatgcgttgcgctcagcactcaaccccagagtgtagcttaggtcgatga | |
| | gtctggacactcctcaccggcgacggtggtccaggctgcgttggcggcctacctgtggtccaa | |
| | agccacaggacgctagttgtgaacaaggtgtgaagagcctattgagctacaagagaatcctcc | |
| | ggcccctgaatgcggctaatcctaaccacggagcaagggtacacaaaccagtgtatatcttgtc | |
| | gtaacgcgcaagtctgtggcggaaccgactactttgggtgtccgtgtttccttttgtttttatcatgg | |
| | ctgcttatggtgacaatctaagattgttatcatatagctgttggattggccatccggtaatttattgag | |
| | atttgagcatttgcttgtttcttcaacaatttcacctattcattgcatttcagcagtcaaa | |
| iEV-D111 IRES | ttcccaccctagaggcccacgtggcggctagtactctggtattacggtacctttgtacgcctgtttt | 91 |
| | atatccctttccccattgtaacttagaagtttcaaagtacagttcaatagacagtagtacaaaccag | |
| | tactgctacgaacaagcacttctgttgccccggtgtagttgcatagactgtacccacggttgaaa | |
| | gcaactgatccgttatccgctttagtacttcgagaagcctagtagcaccttgggatcttcgatgcg | |
| | ttgcgctcagcactcaaccccagagtgtagcttgggtcgatgagtctggacattcctcaccggc | |
| | gacggtggtccaggctgcgttggcggcctacctgtggcccaaagccacaggacgctagttgt | |
| | gaacaaggtgtgaagagcctattgagctactaaagagtcctccggcccctgaatgcggctaatc | |
| | ctaaccacggagcaagtgctcacagtccagtgagtagcttgtcgtaatgagcaattctgtggcg | |
| | gaaccgactactttgggtgtccgtgtttccttttatttttatcattggctgcttatggtgacaatctaag | |
| | gttgccatcatatagcttttggattggccatcccggtgatatttcgaattattcaatttgctgtttgca | |
| | gctccttttaaacagtattcactcaccagcattcatattttactcttgagttattaacaaatagtcaaa | |
| | a | |
| iHCosV-B1 | ttgccagttttttgaaacgtgtggtttgacgtctaaccattatggcagtgggtggagtactgcaaag | 92 |
| IRES | atgtcaccgtgctttacacggtttttgaaccccacaccggctgtttggcgcttgcaggacagcag | |
| | gtttattttcttttaactctctctttctagccacacacgatctatgtgtgtgggcggagtgatactccc | |
| | gttccttcttggacaggcggcctccacgccctttgtggatcttaaggctaccaagtcactggtgtt | |
| | ggaaagtgaagagaaaggagttccttgggaactacatgtggcattgacagaggttgtagcgat | |
| | gctgtgtgtgtgtgcggattacccccgtggcgacacggaccccacaggccaaaagccctgtcc | |
| | gaaaggacccacacagtggagcaaccccagctcccctcttcaatgttttgtgttagcaaccttgg | |
| | tattattttctctcaagcttccaatacaccgggccccaaagatgtcctgaaggtaccccgtgtatct | |
| | gaggatgagcaccatcaactacccggacttgttctttcgagaacagacgcatgtggtaacccag | |
| | ccccgatcctaaggggtcggggcttttgctcactcagcacaggatctgatcaggagacctcccc | |
| | cccctgctttacagggggcgggggtttaaaaattgcccaaggcctggcaaataacctagggga | |
| | ctaggttttcctttttattttaaagttgtcaat | |
| iEcho-E7 IRES | ttcccacccacagggcccactgggcgctagcacactggtatcacggtacctttgtgcgcctgttt | 93 |
| | tatccaccctcccccaatgtaacttagaagcacaccaaacgcggtcaataggcggctcagtaca | |
| | ccaactgagtctcgaccaagcacttctgttaccccggactgagtatcaataggctgctcacgcg | |
| | gctgaaggagaaaacgttcgttacccggccaattacttcgagaaacctagtaccaccatgaag | |
| | gttgcgcggcgtttcgctccacacaaccccagtgtagatcaggtcgatgagccaccgcattccc | |
| | cacgggcgaccgtggcggtggctgcgctggcggcctgcccatggggcaacccatgggacg | |
| | cttcaatactgacatggtgtgaagagtctattgagctaattggtagtcctccggcccctgaatgcg | |
| | gctaatcctaactgcggagcagacacccacatgccagtgggcagtctgtcgtaacgggtaact | |
| | ctgcagcggaaccgactactttgggtgtccgtgtttccttttattctcatactggctgcttatggtga | |
| | caattgaaagattgttgccatatagctattggattggccatccggtgacaaacagagcggttgttt | |
| | acctatttgtcggttttataccattgaattacaaggttctgaaaaccctcaatttcattttagcacttaa | |
| | taccacaaa | |
| iEcho-E9 IRES | ttaaaacagcctgtgggttgttcccacccacagggcccattgggcgctagcacactggtatcac | 94 |
| | ggtacccttgtgcgcctgttttatccacccctccccatcgtaacctagaagcatgactccaacggt | |
| | caatagacagctcagtaaaccaattgagccttgaccaagcacttctgttaccccggactgagtat | |
| | caataggctgctcacgcggccgaaggagaaaatgttcgttacccggccaattacttcgagaaa | |
| | cctagtaccaccatgaaggttgcgcggtgtttcgctcagcacaaccccagtgtagatcaggccg | |
| | atgagtcaccgcattccccacgggtgaccgtggcggtggctgcgctggcggcctgcccatgg | |
| | ggcaacccatgggacgcttcaatactgacatggtgtgaagagtctattgagctaattggtagtcc | |
| | tccggcccctgaatgcggctaatcctaactgcggagcagatacccacgcaccagtgggcagt | |
| | ctgtcgtaacgggcaactctgcagcggaaccgactactttgggtgtccgtgtttccttttattcttgt | |
| | actggctgcttatggtgacaattgagagattgttaccatatagctattggattggccatccagtgtc | |
| | aaacagagcgattgtctacttgttcattggttttataccattgagttacaaggttctagccactcttaa | |
| | cttcattttaacactcaatacatcaaa | |
| iPV3_ | tggggttaaaacagctctggggttgttcccaccccagaggcccacgtggcggctagtactctg | 95 |
| PAK1019536 | gtatcatggtacctttgtacgcctgttttatacccctaccccacgtaactttagaagcaactcaaca | |
| IRES | agttcaatagggggggtacaaaccagtaccaccacgaacaagcactcctgttaccccggtgat | |
| | cttgcataagctgtgcccacggctgaaggcgagagatccgttatccgcttgagtacttcgagaa | |
| | gcctagtatcaccttgggatcttcgacgcgttgcgctcagcactccacccgagtgtagcttaggc | |
| | tgatgagcctgggcattccccaccggcgacggtggcccaggctgcgttggcggcctacccat | |
| | ggctaacaccatgggacgctagatgtgaacaaggtgtgaagagcctattgagctactcaagag | |
| | tcctccggcccctgaatgcggctaatcccaaccacggagcaggtgtcttcaacccagaaggta | |
| | gcctgtcgtaacgcgtaagtctgtggcggaaccgactactttgggtgtccgtgtttccttttatcttt | |
| | tatatggctgcttatggtgacaatcacagattgttatcataaagcgatttggattggccatccggtg | |
| | aagtacaaacacattatctacttgtttgttggatttactccactcacacaccccacccccaatataa | |
| | catctattgttttgttGacaagaaattactttcgaa | |
| iPV3_ | gttaaaacagctctggggttgttcccaccccagaggcccaagtggggctagtactccggtatc | 96 |
| NIE1219535 | acggtacccttgtacgcctgttttatactcccttcccccgtaacttagaagagttaaaccaagttca | |
| IRES | atagaagggggtacaaaccagtaccaccacgaacaagcacttctgtttccccggtgatattgtat | |
| | agactgtacccacggtcgaaaatgattgatccgttatccgctctcatacttcgagaagcctagtat | |
| | catcttggaatcttcgatgcgttgcgctcggcactcgatcccggagtgtagcttaggtcgatgag | |
| | tctggacactcctcaccggtgacggtggtccagactgcgttggcggcctacctgtggtccaaag | |
| | ccacaggacgctagttgtgaacaaggtgtgaagagcctattgagctatgagagagtcctccgg | |
| | cccctgaatgcggctaatcccaaccacggagcgagagtgcgcaagccagcgtatatctcgtc | |
| | gtaacgcgcaagtctgtggcggaaccgactactttgggtgtccgtgtttccttttatttttgtattgg | |
| | ctgcttatggtgacaatcacagattgttatcataaagcgaattggattggccatccggtgaaagtg | |
| | aaatacattatttgcttatttattggattcagtccacctaattgctatactcacgaacccaccacagc | |
| | cgttttgttGactgaaagccggtaccaata | |
| iPV3_SWI10947 | ttaaaacagctctggggttgttcccaccccagaggcccacgtggcggctagtactccggtatca | 97 |
| IRES | cggtacccttgtacgcctgttttatactcccttccccgtaacttagaagtatacaaaccaagttcaat | |
| | agaagggggtacaaaccagtaccaccacgaacaagcacttctgtttccccggtgaagttgcat | |
| | agattgtccccacggtcgaaagcgactgatccgttatccgctcttgtacttcgggaagcctagtat | |
| | caccttcggatcttcgatgcgttgcgctcagcactcaaccccagagtgtagcttaggtcgatgag | |
| | tctggacgaacctcactggtgacagtggtccaggctgcgttggcggcctacctgtggcccaaa | |
| | gccacaggacgctagttgtgaacaaggtgtgaagagcctactgagctacatgagagtcctccg | |
| | gcccctgaatgcggctaatcccaaccacggagcaggtggtcacaaaccagtggtttgcctgtc | |
| | gtaacgcgaaagtctgtggcggaaccgactactttgggtgtccgtgtttccttttattcttattatgg | |
| | ctgcttatggtgacaatcttagattgttatcataaagcgaattggattggccatccggggaaaatta | |
| | agcatatcatacacttgtttgttggattcacccctctgattacacatactcttggcttgattaggattg | |
| | ttgcattgataagacactattgttgca | |
| iPV3_FIN84 | gggttgttcccaccccagaggcccacgtggggctagtactccggtatcacggtacccttgtac | 98 |
| IRES | gcctgttttatactcccttccccgtaacttagaagtatacaagccaagttcaatagaagggggtac | |
| | aaaccagtaccaccacgaacaagcacttctgtttccccggtgaagttgcatagattgttcccacg | |
| | gtcgaaagcgactgatccgttacccgctcatgtacttcgggaagcctagtatcaccttcggatctt | |
| | cgatgcgttgcgctcagcactcaaccccagagtgtagcttaggtcgatgagtctggacaaacct | |
| | cactggcgacagtggtccaggctgcgttggcggcctacctgtggcccaaagccacaggacgc | |
| | tagttgtgaacaaggtgtgaagagcctactgagctacatgagagtcctccggcccctgaatgcg | |
| | gctaatctcaaccacggagcaggtggtcacaaaccagtgatttgcctgtcgtaacgcgaaagtc | |
| | tgtggcggaaccgactactttgggtgtccgtgtttccttttactctcattatggctgcttatggtgac | |
| | aatcctagattgttatcataaagcgagttggattggccatccggtgaaaatcaagcacatcatata | |
| | tttgtttgttgggtttaaccctctgattgcacgcactcttggcttggttaggattattgtgtggaaaag | |
| | gcaccatagttaca | |
| Apt-cIF4G | ACTCACTATTTGTTTTCGCGCCCAGTTGCAAAAAGTGTCG | 99 |
| synIRES-RC01 | ttaaaacagcctgtgggttgatcccacccacaggcccattgggcgctagcactctggtatcacg | 100 |
| | gtacctttgtgcgcctgttttataccccctACTCACTATTTGTTTTCGCGCCC | |
| | AGTTGCAAAAAGTGTCGcccccaactgtaacttagaagtaacacacaccgatc | |
| | aacagtcagcgtggcacaccagccacgttttgatcaagcacttctgttaccccggactgagtatc | |
| | aatagactgctcacgcggttgaaggagaaagcgttcgttatccggccaactacttcgaaaaacc | |
| | tagtaacaccgtggaagttgcagagtgtttcgctcagcactaccccagtgtagatcaggtcgatg | |
| | agtcaccgcattccccacgggcgaccgtggcggtggctgcgttggcggcctgcccatgggga | |
| | aacccatgggacgctctaatacagacatggtgcgaagagtctattgagctagttggtagtcctcc | |
| | ggcccctgaatgcggctaatcctaactgcggagcacacaccctcaagccagagggcagtgtg | |
| | tcgtaacgggcaactctgcagcggaaccgactactttgggtgtcgtgtttcattttattcctatact | |
| | ggctgcttatggtgacaattgagagatcgttaccatatagctattggattggccatccggtgacta | |
| | atagagctattatatatccctttgttgggtttataccacttagcttgaaagaggttaaaacattacaat | |
| | tcattgttaagttgaatacagcaaa | |
| synIRES-RC02 | ttaaaacagcctgtgggttgatcccacccacaggcccattgggcgctagcactctggtatcacg | 101 |
| | gtacctttgtgcgcctgttttataccccctcccccaactgtaacttagaagtaacacacaccgatc | |
| | aacagtcagcgtgACTCACTATTTGTTTTCGCGCCCAGTTGCAAA | |
| | AAGTGTCGcacgttttgatcaagcacttctgttaccccggactgagtatcaatagactgct | |
| | cacgcggttgaaggagaaagcgttcgttatccggccaactacttcgaaaaacctagtaacacc | |
| | gtggaagttgcagagtgtttcgctcagcactaccccagtgtagatcaggtcgatgagtcaccgc | |
| | attccccacgggcgaccgtggcggtggctgcgttggcggcctgcccatggggaaacccatgg | |
| | gacgctctaatacagacatggtgcgaagagtctattgagctagttggtagtcctccggcccctga | |
| | atgcggctaatcctaactgcggagcacacaccctcaagccagagggcagtgtgtcgtaacgg | |
| | gcaactctgcagcggaaccgactactttgggtgtcgtgtttcattttattcctatactggctgcttat | |
| | ggtgacaattgagagatcgttaccatatagctattggattggccatccggtgactaatagagctat | |
| | tatatatccctttgttgggtttataccacttagcttgaaagaggttaaaacattacaattcattgttaag | |
| | ttgaatacagcaaa | |
| synIRES-RC03 | ttaaaacagcctgtgggttgatcccacccacaggcccattgggcgctagcactctggtatcacg | 102 |
| | gtacctttgtgcgcctgttttataccccctcccccaactgtaacttagaagtaacacacaccgatc | |
| | aacagtcagcgtggcacaccagccacgttttgatcaagcacttctgttaccACTCACTAT | |
| | TTGTTTTCGCGCCCAGTTGCAAAAAGTGTCGccggactgagtatc | |
| | aatagactgctcacgcggttgaaggagaaagcgttcgttatccggccaactacttcgaaaaacc | |
| | tagtaacaccgtggaagttgcagagtgtttcgctcagcactaccccagtgtagatcaggtcgatg | |
| | agtcaccgcattccccacgggcgaccgtggcggtggctgcgttggcggcctgcccatgggga | |
| | aacccatgggacgctctaatacagacatggtgcgaagagtctattgagctagttggtagtcctcc | |
| | ggcccctgaatgcggctaatcctaactgcggagcacacaccctcaagccagagggcagtgtg | |
| | tcgtaacgggcaactctgcagcggaaccgactactttgggtgtcgtgtttcattttattcctatact | |
| | ggctgcttatggtgacaattgagagatcgttaccatatagctattggattggccatccggtgacta | |
| | atagagctattatatatccctttgttgggtttataccacttagcttgaaagaggttaaaacattacaat | |
| | tcattgttaagttgaatacagcaaa | |
| synIRES-RC04 | ttaaaacagcctgtgggttgatcccacccacaggcccattgggcgctagcactctggtatcacg | 103 |
| | gtacctttgtgcgcctgttttataccccctcccccaactgtaacttagaagtaacacacaccgatc | |
| | aacagtcagcgtggcacaccagccacgttttgatcaagcacttctgttaccccggactgagtatc | |
| | aatagactgcACTCACTATTTGTTTTCGCGCCCAGTTGCAAAAA | |
| | GTGTCGgcggttgaaggagaaagcgttcgttatccggccaactacttcgaaaaacctagt | |
| | aacaccgtggaagttgcagagtgtttcgctcagcactaccccagtgtagatcaggtcgatgagt | |
| | caccgcattccccacgggcgaccgtggcggtggctgcgttggggcctgcccatggggaaa | |
| | cccatgggacgctctaatacagacatggtgcgaagagtctattgagctagttggtagtcctccg | |
| | gcccctgaatgcggctaatcctaactgcggagcacacaccctcaagccagagggcagtgtgt | |
| | cgtaacgggcaactctgcagcggaaccgactactttgggtgtcgtgtttcattttattcctatactg | |
| | gctgcttatggtgacaattgagagatcgttaccatatagctattggattggccatccggtgactaat | |
| | agagctattatatatccctttgttgggtttataccacttagcttgaaagaggttaaaacattacaattc | |
| | attgttaagttgaatacagcaaa | |
| | ttaaaacagcctgtgggttgatcccacccacaggcccattgggcgctagcactctggtatcacg | |
| synIRES-RC05 | gtacctttgtgcgcctgttttataccccctcccccaactgtaacttagaagtaacacacaccgatc | 104 |
| | aacagtcagcgtggcacaccagccacgttttgatcaagcacttctgttaccccggactgagtatc | |
| | aatagactgctcacgcggttgaaggagaaagcgttcgttatACTCACTATTTGTTT | |
| | TCGCGCCCAGTTGCAAAAAGTGTCGccggccaactacttcgaaaaacc | |
| | tagtaacaccgtggaagttgcagagtgtttcgctcagcactaccccagtgtagatcaggtcgatg | |
| | agtcaccgcattccccacgggcgaccgtggcggtggctgcgttggcggcctgcccatgggga | |
| | aacccatgggacgctctaatacagacatggtgcgaagagtctattgagctagttggtagtcctcc | |
| | ggcccctgaatgcggctaatcctaactgcggagcacacaccctcaagccagagggcagtgtg | |
| | tcgtaacgggcaactctgcagcggaaccgactactttgggtgtcgtgtttcattttattcctatact | |
| | ggctgcttatggtgacaattgagagatcgttaccatatagctattggattggccatccggtgacta | |
| | atagagctattatatatccctttgttgggtttataccacttagcttgaaagaggttaaaacattacaat | |
| | tcattgttaagttgaatacagcaaa | |
| synIRES-RC06 | ttaaaacagcctgtgggttgatcccacccacaggcccattgggcgctagcactctggtatcacg | 105 |
| | gtacctttgtgcgcctgttttataccccctcccccaactgtaacttagaagtaacacacaccgatc | |
| | aacagtcagcgtggcacaccagccacgttttgatcaagcacttctgttaccccggactgagtatc | |
| | aatagactgctcacgcggttgaaggagaaagcgttcgttatccggccaactacttcgaaaaacc | |
| | tagtaacaccgtggaagttgcagagtgtttcgctcagcactACTCACTATTTGTTT | |
| | TCGCGCCCAGTTGCAAAAAGTGTCGagtgtagatcaggtcgatgagtca | |
| | ccgcattccccacgggcgaccgtggcggtggctgcgttggcggcctgcccatggggaaacc | |
| | catgggacgctctaatacagacatggtgcgaagagtctattgagctagttggtagtcctccggc | |
| | ccctgaatgcggctaatcctaactgcggagcacacaccctcaagccagagggcagtgtgtcgt | |
| | aacgggcaactctgcagcggaaccgactactttgggtgtcgtgtttcattttattcctatactggct | |
| | gcttatggtgacaattgagagatcgttaccatatagctattggattggccatccggtgactaatag | |
| | agctattatatatccctttgttgggtttataccacttagcttgaaagaggttaaaacattacaattcatt | |
| | gttaagttgaatacagcaaa | |
| synIRES-RC07 | ttaaaacagcctgtgggttgatcccacccacaggcccattgggcgctagcactctggtatcacg | 106 |
| | gtacctttgtgcgcctgttttataccccctcccccaactgtaacttagaagtaacacacaccgatc | |
| | aacagtcagcgtggcacaccagccacgttttgatcaagcacttctgttaccccggactgagtatc | |
| | aatagactgctcacgcggttgaaggagaaagcgttcgttatccggccaactacttcgaaaaacc | |
| | tagtaacaccgtggaagttgcagagtgtttcgctcagcactaccccagtgtagatcaggtcgatg | |
| | agtcaccgcACTCACTATTTGTTTTCGCGCCCAGTTGCAAAAA | |
| | GTGTCGgcggtggctgcgttggcggcctgcccatggggaaacccatgggacgctctaat | |
| | acagacatggtgcgaagagtctattgagctagttggtagtcctccggcccctgaatgcggctaa | |
| | tcctaactgcggagcacacaccctcaagccagagggcagtgtgtcgtaacgggcaactctgc | |
| | agcggaaccgactactttgggtgtcgtgtttcattttattcctatactggctgcttatggtgacaatt | |
| | gagagatcgttaccatatagctattggattggccatccggtgactaatagagctattatatatccct | |
| | ttgttgggtttataccacttagcttgaaagaggttaaaacattacaattcattgttaagttgaatacag | |
| | caaa | |
| synIRES-RC08 | ttaaaacagcctgtgggttgatcccacccacaggcccattgggcgctagcactctggtatcacg | 107 |
| | gtacctttgtgcgcctgttttataccccctcccccaactgtaacttagaagtaacacacaccgatc | |
| | aacagtcagcgtggcacaccagccacgttttgatcaagcacttctgttaccccggactgagtatc | |
| | aatagactgctcacgcggttgaaggagaaagcgttcgttatccggccaactacttcgaaaaacc | |
| | tagtaacaccgtggaagttgcagagtgtttcgctcagcactaccccagtgtagatcaggtcgatg | |
| | agtcaccgcattccccacgggcgaccgtggcggtggctgcgttggcggcctgcccatgggA | |
| | CTCACTATTTGTTTTCGCGCCCAGTTGCAAAAAGTGTCGcc | |
| | catgggacgctctaatacagacatggtgcgaagagtctattgagctagttggtagtcctccggc | |
| | ccctgaatgcggctaatcctaactgcggagcacacaccctcaagccagagggcagtgtgtcgt | |
| | aacgggcaactctgcagcggaaccgactactttgggtgtcgtgtttcattttattcctatactggct | |
| | gcttatggtgacaattgagagatcgttaccatatagctattggattggccatccggtgactaatag | |
| | agctattatatatccctttgttgggtttataccacttagcttgaaagaggttaaaacattacaattcatt | |
| | gttaagttgaatacagcaaa | |
| synIRES-RC09 | ttaaaacagcctgtgggttgatcccacccacaggcccattgggcgctagcactctggtatcacg | 108 |
| | gtacctttgtgcgcctgttttataccccctcccccaactgtaacttagaagtaacacacaccgatc | |
| | aacagtcagcgtggcacaccagccacgttttgatcaagcacttctgttaccccggactgagtatc | |
| | aatagactgctcacgcggttgaaggagaaagcgttcgttatccggccaactacttcgaaaaacc | |
| | tagtaacaccgtggaagttgcagagtgtttcgctcagcactaccccagtgtagatcaggtcgatg | |
| | agtcaccgcattccccacgggcgaccgtggcggtggctgcgttggcggcctgcccatgggga | |
| | aacccatgggacgctctaatacagacatggtgcgaagagtctattgagctagttggtagtcAC | |
| | TCACTATTTGTTTTCGCGCCCAGTTGCAAAAAGTGTCGCC | |
| | CCctccggcccctgaatgcggctaatcctaactgcggagcacacaccctcaagccagaggg | |
| | cagtgtgtcgtaacgggcaactctgcagcggaaccgactactttgggtgtcgtgtttcattttattc | |
| | ctatactggctgcttatggtgacaattgagagatcgttaccatatagctattggattggccatccgg | |
| | tgactaatagagctattatatatccctttgttgggtttataccacttagcttgaaagaggttaaaacat | |
| | tacaattcattgttaagttgaatacagcaaa | |
| synIRES-RC10 | ttaaaacagcctgtgggttgatcccacccacaggcccattgggcgctagcactctggtatcacg | 109 |
| | gtacctttgtgcgcctgttttataccccctcccccaactgtaacttagaagtaacacacaccgatc | |
| | aacagtcagcgtggcacaccagccacgttttgatcaagcacttctgttaccccggactgagtatc | |
| | aatagactgctcacgcggttgaaggagaaagcgttcgttatccggccaactacttcgaaaaacc | |
| | tagtaacaccgtggaagttgcagagtgtttcgctcagcactaccccagtgtagatcaggtcgatg | |
| | agtcaccgcattccccacgggcgaccgtggcggtggctgcgttggcggcctgcccatgggga | |
| | aacccatgggacgctctaatacagacatggtgcgaagagtctattgagctagttggtagtcctcc | |
| | ggcccctgaatgcggctaatcctaactgcggagcacacaccctcACTCACTATTTG | |
| | TTTTCGCGCCCAGTTGCAAAAAGTGTCGgagggcagtgtgtcgtaac | |
| | gggcaactctgcagcggaaccgactactttgggtgtcgtgtttcattttattcctatactggctgctt | |
| | atggtgacaattgagagatcgttaccatatagctattggattggccatccggtgactaatagagct | |
| | attatatatccctttgttgggtttataccacttagcttgaaagaggttaaaacattacaattcattgtta | |
| | agttgaatacagcaaa | |
| | ttaaaacagcctgtgggttgatcccacccacaggcccattgggcgctagcactctggtatcacg | |
| synIRES-RC11 | gtacctttgtgcgcctgttttataccccctcccccaactgtaacttagaagtaacacacaccgatc | 110 |
| | aacagtcagcgtggcacaccagccacgttttgatcaagcacttctgttaccccggactgagtatc | |
| | aatagactgctcacgcggttgaaggagaaagcgttcgttatccggccaactacttcgaaaaacc | |
| | tagtaacaccgtggaagttgcagagtgtttcgctcagcactaccccagtgtagatcaggtcgatg | |
| | agtcaccgcattccccacgggcgaccgtggcggtggctgcgttggcggcctgcccatgggga | |
| | aacccatgggacgctctaatacagacatggtgcgaagagtctattgagctagttggtagtcctcc | |
| | ggcccctgaatgcggctaatcctaactgcggagcacacaccctcaagccagagggcagtgtg | |
| | tcgtaacgggcaactctgcagcggaaccgactactttgggtgtcgtgtttcattttACTCAC | |
| | TATTTGTTTTCGCGCCCAGTTGCAAAAAGTGTCGattcctatact | |
| | ggctgcttatggtgacaattgagagatcgttaccatatagctattggattggccatccggtgacta | |
| | atagagctattatatatccctttgttgggtttataccacttagcttgaaagaggttaaaacattacaat | |
| | tcattgttaagttgaatacagcaaa | |
| synIRES-RC12 | ttaaaacagcggatgggtaccccaccatccgacccactgggtgtagtactctggtacttcgtacc | 111 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggCACTCACTATTTGTTTT | |
| | CGCGCCCAGTTGCAAAAAGTGTCGGCCagtttggtcgatgaggctag | |
| | gaattccccacgggtgaccgtgtcctagcctgcgtggcggccaacccagcttatgctgggacg | |
| | cctttttatagacatggtgtgaagactcgcatgtgcttggttgtgattcctccggcccctgaatgcg | |
| | gctaaccttaaccctggagccttgtgtcacaaaccagtgatgataaggtcgtaatgagcaattcc | |
| | gggacgggaccgactactttgggtgtccgtgtttcttatttttcttattattgtcttatggtcacagcat | |
| | atatataacatatactgtgatc | |
| synIRES-RC13 | ttaaaacagcggatgggtaccccaccatccgacccactgggtgtagtactctggtacttcgtacc | 112 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggCGACTCACTATTTGTTT | |
| | TCGCGCCCAGTTGCAAAAAGTGTCGCGCCagtttggtcgatgaggct | |
| | aggaattccccacgggtgaccgtgtcctagcctgcgtggcggccaacccagcttatgctggga | |
| | cgcctttttatagacatggtgtgaagactcgcatgtgcttggttgtgattcctccggcccctgaatg | |
| | cggctaaccttaaccctggagccttgtgtcacaaaccagtgatgataaggtcgtaatgagcaatt | |
| | ccgggacgggaccgactactttgggtgtccgtgtttcttatttttcttattattgtcttatggtcacag | |
| | catatatataacatatactgtgatc | |
| synIRES-RC14 | ttaaaacagcggatgggtaccccaccatccgacccactgggtgtagtactctggtacttcgtacc | 113 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggCGGACTCACTATTTGTT | |
| | TTCGCGCCCAGTTGCAAAAAGTGTCGCCGCCagtttggtcgatga | |
| | ggctaggaattccccacgggtgaccgtgtcctagcctgcgtggcggccaacccagcttatgct | |
| | gggacgcctttttatagacatggtgtgaagactcgcatgtgcttggttgtgattcctccggcccct | |
| | gaatgcggctaaccttaaccctggagccttgtgtcacaaaccagtgatgataaggtcgtaatga | |
| | gcaattccgggacgggaccgactactttgggtgtccgtgtttcttatttttcttattattgtcttatggt | |
| | cacagcatatatataacatatactgtgatc | |
| synIRES-RC15 | ttaaaacagcggatgggtaccccaccatccgacccactgggtgtagtactctggtacttcgtacc | 114 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggaACTCACTATTTGTT | |
| | TTCGCGCCCAGTTGCAAAAAGTGTCGtccactagtttggtcgatgaggc | |
| | taggaattccccacgggtgaccgtgtcctagcctgcgtggcggccaacccagcttatgctggg | |
| | acgcctttttatagacatggtgtgaagactcgcatgtgcttggttgtgattcctccggcccctgaat | |
| | gcggctaaccttaaccctggagccttgtgtcacaaaccagtgatgataaggtcgtaatgagcaa | |
| | ttccgggacgggaccgactactttgggtgtccgtgtttcttatttttcttattattgtcttatggtcaca | |
| | gcatatatataacatatactgtgatc | |
| synIRES-RC16 | ttaaaacagcggatgggtaccccaccatccgacccactgggtgtagtactctggtacttcgtacc | 115 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggaGACTCACTATTTGT | |
| | TTTCGCGCCCAGTTGCAAAAAGTGTCGCtccactagtttggtcgatga | |
| | ggctaggaattccccacgggtgaccgtgtcctagcctgcgtggcggccaacccagcttatgct | |
| | gggacgcctttttatagacatggtgtgaagactcgcatgtgcttggttgtgattcctccggcccct | |
| | gaatgcggctaaccttaaccctggagccttgtgtcacaaaccagtgatgataaggtcgtaatga | |
| | gcaattccgggacgggaccgactactttgggtgtccgtgtttcttatttttcttattattgtcttatggt | |
| | cacagcatatatataacatatactgtgatc | |
| synIRES-RC17 | ttaaaacagcggatgggtaccccaccatccgacccactgggtgtagtactctggtacttcgtacc | 116 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggaGGACTCACTATTTG | |
| | TTTTCGCGCCCAGTTGCAAAAAGTGTCGCCtccactagtttggtcga | |
| | tgaggctaggaattccccacgggtgaccgtgtcctagcctgcgtggcggccaacccagcttat | |
| | gctgggacgcctttttatagacatggtgtgaagactcgcatgtgcttggttgtgattcctccggcc | |
| | cctgaatgcggctaaccttaaccctggagccttgtgtcacaaaccagtgatgataaggtcgtaat | |
| | gagcaattccgggacgggaccgactactttgggtgtccgtgtttcttatttttcttattattgtcttatg | |
| | gtcacagcatatatataacatatactgtgatc | |
| synIRES-RC18 | ttaaaacagcggatgggtaccccaccatccgacccactgggtgtagtactctggtacttcgtacc | 117 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaACTCACTATTTGTTTTCGCGCCCAGTTGCAAAA | |
| | AGTGTCGtcctagcctgcgtggcggccaacccagcttatgctgggacgcctttttatagac | |
| | atggtgtgaagactcgcatgtgcttggttgtgattcctccggcccctgaatgcggctaaccttaac | |
| | cctggagccttgtgtcacaaaccagtgatgataaggtcgtaatgagcaattccgggacgggac | |
| | cgactactttgggtgtccgtgtttcttatttttcttattattgtcttatggtcacagcatatatataacata | |
| | tactgtgatc | |
| synIRES-RC19 | ttaaaacagcggatgggtaccccaccatccgacccactgggtgtagtactctggtacttcgtacc | 118 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaattccccacggACTCACTATTTGTTTTCGCGCCCAGTT | |
| | GCAAAAAGTGTCGccgtgtcctagcctgcgtggcggccaacccagcttatgctgg | |
| | gacgcctttttatagacatggtgtgaagactcgcatgtgcttggttgtgattcctccggcccctga | |
| | atgcggctaaccttaaccctggagccttgtgtcacaaaccagtgatgataaggtcgtaatgagc | |
| | aattccgggacgggaccgactactttgggtgtccgtgtttcttatttttcttattattgtcttatggtca | |
| | cagcatatatataacatatactgtgatc | |
| synIRES-RC20 | ttaaaacagcggatgggtaccccaccatccgacccactgggtgtagtactctggtacttcgtacc | 119 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaattccccacgggtgaccgtgtcctagcctgcgtggcggccaacccACTC | |
| | ACTATTTGTTTTCGCGCCCAGTTGCAAAAAGTGTCGgggacg | |
| | cctttttatagacatggtgtgaagactcgcatgtgcttggttgtgattcctccggcccctgaatgcg | |
| | gctaaccttaaccctggagccttgtgtcacaaaccagtgatgataaggtcgtaatgagcaattcc | |
| | gggacgggaccgactactttgggtgtccgtgtttcttatttttcttattattgtcttatggtcacagcat | |
| | atatataacatatactgtgatc | |
| synIRES-RC21 | ttaaaacagcggatgggtaccccaccatccgacccactgggtgtagtactctggtacttcgtacc | 120 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaattccccacgggtgaccgtgtcctagcctgcgtggcggccaacccGACT | |
| | CACTATTTGTTTTCGCGCCCAGTTGCAAAAAGTGTCGCggg | |
| | acgcctttttatagacatggtgtgaagactcgcatgtgcttggttgtgattcctccggcccctgaat | |
| | gcggctaaccttaaccctggagccttgtgtcacaaaccagtgatgataaggtcgtaatgagcaa | |
| | ttccgggacgggaccgactactttgggtgtccgtgtttcttatttttcttattattgtcttatggtcaca | |
| | gcatatatataacatatactgtgatc | |
| synIRES-RC22 | ttaaaacagcggatgggtaccccaccatccgacccactgggtgtagtactctggtacttcgtacc | 121 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaattccccacgggtgaccgtgtcctagcctgcgtggcggccaacccGGAC | |
| | TCACTATTTGTTTTCGCGCCCAGTTGCAAAAAGTGTCGCC | |
| | gggacgcctttttatagacatggtgtgaagactcgcatgtgcttggttgtgattcctccggcccct | |
| | gaatgcggctaaccttaaccctggagccttgtgtcacaaaccagtgatgataaggtcgtaatga | |
| | gcaattccgggacgggaccgactactttgggtgtccgtgtttcttatttttcttattattgtcttatggt | |
| | cacagcatatatataacatatactgtgatc | |
| synIRES-RC23 | ttaaaacagcggatgggtaccccaccatccgacccactgggtgtagtactctggtacttcgtacc | 122 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaattccccacgggtgaccgtgtcctagcctgcgtggcggccaacccagcAC | |
| | TCACTATTTGTTTTCGCGCCCAGTTGCAAAAAGTGTCGgctg | |
| | ggacgcctttttatagacatggtgtgaagactcgcatgtgcttggttgtgattcctccggcccctg | |
| | aatgcggctaaccttaaccctggagccttgtgtcacaaaccagtgatgataaggtcgtaatgag | |
| | caattccgggacgggaccgactactttgggtgtccgtgtttcttatttttcttattattgtcttatggtc | |
| | acagcatatatataacatatactgtgatc | |
| synIRES-RC24 | ttaaaacagcggatgggtaccccaccatccgacccactgggtgtagtactctggtacttcgtacc | 123 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaattccccacgggtgaccgtgtcctagcctgcgtggcggccaacccagcCA | |
| | CTCACTATTTGTTTTCGCGCCCAGTTGCAAAAAGTGTCGG | |
| | gctgggacgcctttttatagacatggtgtgaagactcgcatgtgcttggttgtgattcctccggcc | |
| | cctgaatgcggctaaccttaaccctggagccttgtgtcacaaaccagtgatgataaggtcgtaat | |
| | gagcaattccgggacgggaccgactactttgggtgtccgtgtttcttatttttcttattattgtcttatg | |
| | gtcacagcatatatataacatatactgtgatc | |
| synIRES-RC25 | ttaaaacagcggatgggtaccccaccatccgacccactgggtgtagtactctggtacttcgtacc | 124 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggatttcccctccactagtttggtcgat | |
| | gaggctaggaattccccacgggtgaccgtgtcctagcctgcgtggcggccaacccagcCC | |
| | ACTCACTATTTGTTTTCGCGCCCAGTTGCAAAAAGTGTCG | |
| | GGgctgggacgcctttttatagacatggtgtgaagactcgcatgtgcttggttgtgattcctccg | |
| | gcccctgaatgcggctaaccttaaccctggagccttgtgtcacaaaccagtgatgataaggtcg | |
| | taatgagcaattccgggacgggaccgactactttgggtgtccgtgtttcttatttttcttattattgtct | |
| | tatggtcacagcatatatataacatatactgtgatc | |
| synIRES-RC26 | ttaaaacagcggatgggtaccccaccatccgacccactgggtgtagtactctggtacttcgtacc | 125 |
| | tttgtacgcctgttcttcccattgtacccttcctgaacttccaacccaagtaacgttagaagctcaac | |
| | atttagtacaacaggaagcaccacatccagtggtgtttagtacaagcacttctgtttccccggag | |
| | cgaggtataggctgtacccactgccaaaaacctttaaccgttatccgccaaccaactacgtaaa | |
| | agctagtagtattatgtttttaactaggcgttcgatcaggtggaACTCACTATTTGTT | |
| | TTCGCGCCCAGTTGCAAAAAGTGTCGtccactagtttggtcgatgaggc | |
| | taggaattccccacgggtgaccgtgtcctagcctgcgtggcggccaacccagcACTCAC | |
| | TATTTGTTTTCGCGCCCAGTTGCAAAAAGTGTCGgctgggacg | |
| | cctttttatagacatggtgtgaagactcgcatgtgcttggttgtgattcctccggcccctgaatgcg | |
| | gctaaccttaaccctggagccttgtgtcacaaaccagtgatgataaggtcgtaatgagcaattcc | |
| | gggacgggaccgactactttgggtgtccgtgtttcttatttttcttattattgtcttatggtcacagcat | |
| | atatataacatatactgtgatc | |

1. A circular RNA molecule comprising an internal ribosome entry site (IRES) sequence operably linked to a protein-coding sequence;
wherein the IRES sequence is a viral sequence of a Type 1 IRES, an enterovirus IRES, or a human rhinovirus (HRV) IRES; and
wherein the protein-coding sequence encodes a non-viral protein.

2. (canceled)

3. The circular RNA molecule of claim 1, wherein the non-viral protein is a human protein.

4-7. (canceled)

8. The circular RNA molecule of claim 1, wherein the IRES is any one of the following IRES: iEMCV, iHCV, iCVB5, i Swine Vesicular, iHRV-A2, iHRV-C3, iHRV-Cll, iCVBl, iPV2, iHRV-B17, iEchoV-E15, iEV71, iHRV-A9, iSiminanV4, iEV-D94, iSimianA5, iPV3, iHRV-C54, iHRV-AlOO, iHRV-B37, iHRV-B4, iHRV-B92, iHRV-B3, iHRV-Al, iEV107, iEV-B83, iHRV-A57, iHRV-B35, iHRV-B4, iEV-D68, iHRVB R93, iHRV B5, iHRVB-B52, iHRVB-B93, iHRV-B84, iHRV-B83 SC2220, iHRV-B72, iHRV-B69, iHRVB SC0739, iHRV-B91, iHRV-B42, iHRV-B6, iHRV-B83, iHRV-B48, iHRV-B99, iHRV-B79, iHRV-B97, iHRV-B27, iHRVB 3039, iHRVB-B14, iCosV-B1, or a fragment or derivative thereof.

9. (canceled)

10. The circular RNA molecule of claim 1, wherein the IRES is iCVB3, iHRV-B3, or a fragment or derivative thereof.

11. (canceled)

12. A circular RNA molecule comprising a synthetic internal ribosome entry site (IRES) sequence operably linked to a protein-coding sequence, wherein the synthetic IRES sequence comprises at least one aptamer.

13.-16. (canceled)

17. The circular RNA molecule of claim 12, wherein the aptamer is modified to have an extended stem region.

18. The circular RNA molecule of claim 12, wherein the aptamer is positioned within the secondary structure of the IRES so that is spatially proximal to portion of the IRES responsible for translation initiation.

19. The circular RNA molecule of claim 12, wherein the aptamer does not interrupt the native eIF4G binding site of the IRES and does not interrupt a native GRNA tetraloop within the IRES.

20. The circular RNA molecule of claim 12, wherein the aptamer is an eIF4G-binding aptamer.

21. The circular RNA molecule of claim 20, wherein the eIF4G-binding aptamer is encoded by the sequence of SEQ ID NO: 99.

22. (canceled)

23. The circular RNA of claim 12, wherein the synthetic IRES is a modified enterovirus IRES, a modified human rhinovirus (HRV) IRES, a modified iCVB3 IRES, or a modified iHRV-B3 IRES.

24. (canceled)

25. A circular RNA molecule comprising a modified iCVB3 IRES or a modified iHRV-B3 IRES, wherein the modified iCVB3 IRES or the modified iHRV-B3 IRES comprises an aptamer inserted in domain I, II, III, IV, V, VI or VII thereof.

26. (canceled)

27. The circular RNA molecule of claim 25, wherein the modified iCVB3 IRES comprises an aptamer inserted in domain IV thereof.

28.-32. (canceled)

33. The circular RNA molecule of claim 25, wherein the modified iHRV-B3 IRES comprises an aptamer inserted in domain IV thereof.

34.-36. (canceled)

37. The circular RNA molecule of claim 1, wherein said circular RNA molecule comprises at least one 2-thiouridine (2ThioU) or at least one 2′-0-methylcitidine (20MeC).

38-43. (canceled)

44. The circular RNA molecule of claim 1, wherein said molecule comprises a nucleic acid spacer upstream of said IRES.

45. A nucleic acid that encodes the circular RNA molecule of claim 1.

46. A composition comprising the circular RNA molecule of claim 1.

47. (canceled)

48. A method of producing a protein in a cell, the method comprising contacting a cell with the circular RNA molecule of claim 1 under conditions whereby the protein-coding nucleic acid sequence of the circular RNA is translated and the protein is produced in the cell.

49. A method of producing a protein in vitro, the method comprising contacting a cell-free extract with the circular RNA molecule of claim 1 under conditions whereby the protein-coding nucleic acid sequence of the circular RNA is translated and the protein is produced.

50. (canceled)
